SEQUENCE LISTING (102 sequences)

SEQ ID NO: 1 - DNA, 1104 bp, Artificial Sequence
  Description of Artificial Sequence Synthetic polynucleotide
atgtcatccgattctagctctataggggcgatcgaaaccagaatacgtgaactggtccatgactatgtgggtgtcaatggcactgatgcacctataacgccagctttacgtcccatgtttcataccgtcgttgaccaggcgcttgcttcgagcgagggagggaaaagattacgcgctcttttaactttggacgcatatgatgtcttggcaggggcgccggattctactcaaagtaggtccgtcagaactaaggtcctagatttcgcgtgcgctatcgaggtcttccaaaccgcggcgttggtacacgatgacctgattgatgatagcgacttgaggaggggcaaaccttctgcacattgcgcactaacatcatttgcaggagcaaggagcataggtcgtggactgggccttatgcttggagatatgttggctacggcatgtacgctgataatggaagacgctagtactggtatggtcgagcaccgtaggctggtcgaagcgtttctaagtatgcagcacgacgtcgaagttggacaagtgttggatttagctatcgaaagaatgcccctggacgacccacaggcgcttgcagaagccagccttgacgtctttcgttggaaaactgcgtcctacacgaccatagcaccactaatgttggctttcttagcaagtggtatgacaagcgaagccgcgaaccttcactgtcatgctattggattgccgttaggccaagcattccagcttgcagacgatctgttggacgttacaggaagttctcgttctaccgggaaacccgtgggtggtgatattagagaaggtaaaagaacagtattacttgcagacgcgatgatgctagggaccgctgcacagcgtgtccaactacagcaattatatgagcaacccttcagatcagatgcgcaggttcatgagaccattgctctattccatgataccggcgcgattgaacactcacatgagagaatagctaagttgtggagtcaaacccaagagtctattgaggctatgggccttacagccgctcagagtcagagcctgcgtaaggcgtgcgagcgtttcctaccggattttaccgccgaaaggtaa

SEQ ID NO: 2 - DNA, 1086 bp, Artificial Sequence
  Description of Artificial Sequence Synthetic polynucleotide
atgtcatgtaccactgctaataatcgtgagatcatcgaacccaggatcatacaattagtcagggaacttaccgcggcaccggcgaccgacgaagttgccgacgcgttgaagccggtaatggaacaagtcgtagaccaggccgccagttcttcccaaggcgggaagagactaagggcccttttagcattagacgccttcgatattcttgcaggtgacgtaacgccagataggcgtgatgcaatgattgatctagcatgtgcaatcgaagtgttccaaactgcggcgctggttcacgatgacattatagacgaaagcgacctacgtcgtggcaaaccctcagcacaccatgctcttgagcaagcagtccatagcggcgcgataggcagaggtttgggtctgatgttgggagacatccttgcaaccgcatgcatagaaattactcgtagaagcgcctcacgtcttcctaacactgacgccttgaatgaggcgttcctaacaatgcagagagaagtagaaattggtcaggtactagacttagccgtggagatgactcctctgtctaatccggaagcactagctaacgcaagcctaaatgtgtttaggtggaagaccgcttcatatacgacgatagcacctctattattagcattacttgctgccggtgaatctccagatcaagctaggcactgcgccttagcggtcgggaggcctctggggttggcctttcaattagcggacgatctgctagacgtagtagggtctagcagaaataccggcaaaccagtagggggtgacattagggaaggtaagagaacagtgttgttggccgacgccttgtcagcggctgacacggctgacaaagcggatcttatagcgattttcgaggaggactgtaggaacgataaccaggtggcgagaacgatcgaattatttacatcaacaggtgctctggatcgtagtcgtgagcgtatagctgcattgtggggtgaatcaaggaaagcaatcgctggattggagttgaactccgaggctcaaaggaggctgaccgaggcttgtgcccgttttgtaccggaaagtcttagataa

SEQ ID NO: 3 - DNA, 876 bp, Artificial Sequence
  Description of Artificial Sequence Synthetic polynucleotide
atgtcagataagattaaaaagatgggcgaggaaatagaactttggttaaaagaatatttggataataagggtaactacgataagaagatatatgaagcaatggcttactctttggaggctggcgggaagagaattagaccggtgctgtttctaaacacttactcactatataaggaggattacaagaaagcaatgccgattgcagccgccattgaaatgattcatacatacttcttgatacacgatgatctgccggccatggacaacgacgacttacgaaggggaaaacccactaaccataaaatatttggagaagcaatagcgatacttgcgggagacgctctattaaatgaagcaatgaacataatgtttgagtacagcctgaagaatggggaaaaagcgttaaaagcatgttacaccattgctaaagctgcgggagtcgatgggatgatcggagggcaagtcgtagacattttatcagaagataaatctatctcattggatgagttgtattatatgcacaaaaagaaaaccggtgccttaataaaagcgtcaatacttgctggagccatattgggctcagctacctatactgatatagaactactaggcgagtacggggacaaccttggcttagcgttccagatcaaagatgacatacttgacgtagaaggcgatacaactacccttggcaaaaagacgaaaagcgatgaagataatcacaagacaacctttgttaaagtgtatggaatagagaaatgtaacgaactgtgtactgagatgaccaataagtgttttgacattctaaataagatcaaaaagaatactgataagttgaaagagataacgatgtttcttctgaatagaaactattaa

SEQ ID NO: 4 - DNA, 906 bp, Artificial Sequence
  Description of Artificial Sequence Synthetic polynucleotide
atgtcaaaaaagaggaagaccctggaggacacagcaatgaatatcaacagccttaaagaggaggtggaccaatcattgaaggcatacttcaataaggatcgtgagtataacaaggttttatatgatagcatggcttactcaattaacgtcgggggtaagagaataagacccattctaatgctgttgtcatattacatctataagtctgattataagaaaatccttacaccagcgatggcaatcgaaatgatccacacttacttcattcacgacgacctaccctgtatggacaacgatgatctaaggagaggaaagccgacgaaccataaagtgttcggcgaagcgatagcagtattagcaggggatgccttactaaacgaggcgatgaagatactagtggattactcattggaagaaggtaaaagcgccctgaaggctacgaaaatcatcgccgatgcagcgggatctgatgggatgatcggagggcaaatcgtggacatcataaatgaagataaggaggaaatttctctgaaggaactagactatatgcacctgaagaaaactggcgagttaattaaggctagtataatgagtggtgcagtcttagctgaagcaagtgagggtgacattaaaaagctggaaggttttggttataagctgggactggcttttcaaattaaagatgacatcttagatgtagtgggtaacgcgaaggacttgggtaaaaatgtccataaggaccaggaatccaataaaaacaattacataactatctttggtcttgaagagtgcaagaaaaagtgcgttaatattacagaggagtgcatagaaatcctgtcctccataaaagggaatacggaacccctgaaggtcttgacaatgaaactactagaaaggaaattctaa

SEQ ID NO: 5 - DNA, 903 bp, Artificial Sequence
  Description of Artificial Sequence Synthetic polynucleotide
atgtcagactttcctcagcaattggaggcctgcgtgaaacaggcaaatcaggcgttgtccagattcattgcacccttgccgttccagaatacgcctgtagttgagacgatgcaatacggtgccctacttggtggcaagaggcttcgtccgtttctagtgtacgcaactggacatatgtttggggtatccaccaacacattggacgcgcctgcggctgctgttgagtgcatccatgcctactttttaatccacgacgacctacccgccatggatgatgacgatttaagacgtggtttacctacgtgccacgtcaaattcggagaggctaacgcaattctagccggggatgcccttcagactctggcattttccattctatccgacgccgacatgcccgaggtcagcgaccgtgacaggatttcaatgatctctgaattggcctcagccagcggcatagcaggtatgtgtggaggtcaagccttagacttggatgcggagggaaaacacgttcccttggacgccctggaacgtattcatcgtcacaaaactggggctctaattcgtgctgccgtcaggttgggtgcgcttagtgcaggtgacaagggcaggagagctttacctgtattggataagtatgcggaaagtatcggattagctttccaagtccaagatgacattctggacgtggtcggcgatactgcgactttagggaagaggcagggtgcagaccagcagttggggaagtcaacgtatcctgctctattgggactagaacaagctaggaaaaaggccagggatttgattgatgatgctaggcagtcactaaaacagttggcagagcaatcacttgatacttcagctcttgaggccctggccgattacattatacagagaaataagtaa

SEQ ID NO: 6 - DNA, 885 bp, Artificial Sequence
  Description of Artificial Sequence Synthetic polynucleotide
atgtcaaccaattttagccagcaacatcttccactggtagaaaaggtgatggttgatttcattgcagagtacactgagaacgagagattgaaggaagctatgttgtattccattcacgctggagggaaaaggctgcgtccactgctggtcttaactactgtggccgcctttcagaaagagatggaaactcaagattatcaggtagctgcatccttggaaatgatccatacttatttcctaatacacgacgacctgcccgcgatggatgatgatgatttgagacgtgggaagccgacaaaccacaaggtgtttggggaagccactgctatattagcgggagacggattattaacaggagcctttcagttactatccttgagccaattggggctatccgaaaaggtacttctgatgcagcagctggcgaaagctgctggtaatcagggcatggtatccggacagatgggtgatatagagggggaaaaagtgtctctgacgctggaagagcttgcagcggtacacgagaaaaagactggagcactgatagagtttgcattgattgcaggaggcgtcctagcaaaccaaaccgaggaggttattggtctgcttacgcaattcgcgcatcactatggattggcgttccagatcagggacgacctgcttgatgcgacttcaacggaagccgacttgggcaagaaagttggtcgtgacgaggctctaaataagtccacatatccagcccttttgggaattgcaggtgcaaaagacgctctaacccatcaattagcggagggctccgctgtgctagagaaaattaaggcaaacgttccaaatttctctgaagagcacttggctaatcttcttacccaactgcaattgaggtaa

SEQ ID NO: 7 - DNA, 915 bp, Artificial Sequence
  Description of Artificial Sequence Synthetic polynucleotide
atgtcatcttcccctaatctgtctttctactacaatgaatgtgaaagatttgaatctttccttaaaaatcaccatttgcacctagaaagttttcatccatacttagagaaagcattctttgagatggtactgaatggaggaaagaggttcaggcctaagctattcttggccgtattatgtgcgctagtcggtcagaaggattatagcaaccagcagacggagtattttaagatagcattgagcattgagtgtttgcatacatactttttaatccacgatgatttaccatgtatggataatgctgctttgcgtaggaaccacccgactctacatgctaaatatgatgagaccactgctgtactaataggggacgccctaaacacctactcatttgaactgttgagcaacgctctgcttgaatcccatataatcgtagagctaattaagatactatctgcaaacgggggcataaaaggaatgattctgggacaggcattagattgttatttcgagaacacccccttgaacttggagcagctgactttccttcacgagcacaagactgctaaattaataagtgcaagcctaattatgggactagtcgcaagtggaattaaagacgaggagttgttcaaatggctacaagcgtttggattgaagatgggtctttgttttcaggtgttggacgatatcatagatgtcacacaggacgaagaggagtcaggtaaaactacacacttggattcagctaaaaactccttcgtgaatcttctaggtttggaaagggcgaataattatgcgcaaactctaaagacggaggtcttaaacgacctagacgcactgaagcccgcctatccactgctacaggaaaacctaaatgcgctacttaatacgctgtttaagggtaaaacgtaa

SEQ ID NO: 8 - DNA, 906 bp, Artificial Sequence
  Description of Artificial Sequence Synthetic polynucleotide
atgtcacctataaacgcgaggttaattgcattcgaggatcagtgggttcctgcattaaacgctccgcttaaacaagcgattcttgcagattcccacgacgcacaacttgctgccgctatgacatattctgtcctagcagggggaaaacgtttaaggcccctattaactgtcgcaactatgaggagccttggtgtgacttttgtacctgagagacactggagacccgtaatggcactagagttgctgcatacctactttttgattcatgatgatcttcccgctatggataacgacgcattaaggagaggggaacccaccaatcatgtgaagttcggtgccggtatggccacattggcaggggatgggcttttaacactagcgtttcagtggttgaccgctactgacttgccagcgactatgcaagccgctctagtacaagctctagcaaccgcggcaggcccttcaggcatggtagctggtcaggcgaaagacatacagagcgaacacgtgaatctaccattaagccaacttagagtattacataaagagaaaacaggcgctctactgcattacgccgtgcaggcaggattgatattgggccaagccccagaggcacaatggccagcctacctgcaatttgcggacgcattcggtctagcgttccaaatatatgatgacatattagatgtagtttcatctccggcggagatgggaaaggctacacagaaggatgctgatgaggctaaaaacacatatccgggtaagctgggtctaattggagccaatcaagctctaatagatactatccattctggacaagcagcactgcaaggattaccaacatccacacaaagagatgatctggctgctttcttctcatactttgatacggagagggtcaactaa

SEQ ID NO: 9 - DNA, 861 bp, Artificial Sequence
  Description of Artificial Sequence Synthetic polynucleotide
atgtcagataccaagattttgaaacttgaggacttcctaacagaattttatgagagtgcagagttcccgactgggctggccgaatcagcaaaatacagtctacttgcaggagggaaaagaatacgtccgctattatttttgaacctgctagaagccttcgacttggaactttctaaggctcactaccatgtcgcagcagctttggagatgatacataccggatctcttatccatgacgatcttccagcaatggataatgacgactatagacgtggccaattgacgaatcacaaaaagttcgatgaggcgacagctatcttagctggcgataccttatttttcgatcccttctttattctgtccactgcggatttgagtgcagagataatcgttgccctaacgagagagttggctttcgcctctggctcatacggcatggtcgcggggcaaatcttagatatggcaggtgaaggaaaagaactaacccttgctgaaattgagcaaatccacaggctaaagaccgggcgtctgttgacgttccctttcgtggcagcggggattgtcgcccaaaagagtacggatgaagtcgaaaaactaaggcaagtggggcaaatcttaggacttgctttccaaatcagggacgacatcctggatgttacagcgaccttcgccgagcttggcaaaacccccggcaaggacattttagaggagaagagtacatatgtagctcatttgggcttggaaggagctaaaaagtctttgacggggaacttgtcagaggtgaagaaactacttacagatttatcagtcactgatagtagcgagatttttaagataattgagcaactggaagttaagtaa

SEQ ID NO: 10 - DNA, 918 bp, Artificial Sequence
  Description of Artificial Sequence Synthetic polynucleotide
atgtcaatagatttaaaatctttccaaaaagagtggctaccaaaaataaaccaacaacttgaaaacgaccttagcatggcaagcccagacgcggatctagttgcaatgatgaaatacgctgtcttaaatggtggaaagcgtttgcgtcctttacttactcttgctgtagttacctcattcggggaatccattacaccatccattctgaaggtagcaacagcgattgagtgggtacatagctactttctggtacacgatgatcttccagccatggataacgatatgtttcgtagaggcaaaccttccgtccatgcgctttatggtgaagctaacgcaattttagtaggcgatgcgttattaacgggcgcttttggcgtcatagctaccgctaatagttcttgttccgtcgaagactgcctgcccacagaagagctgcttttgataacccagaacctggcgagagaagccggaggttcaggcatggtcttaggacaattgcatgacatggataaccacactgaagagcagaatgcttctacgaattggctattgaacgatgtgtactcaatgaagacggcagctcttatacgttatacgacgacactaggcgctatcttgacccaccagaacgtcaatgtggaagataatcactttgaccccaaaaaggcaatgtacgactttggggaaaaattcggattagcattccagatacaagatgatcttgatgattaccagcaggaccagcttgaggacgtaaattcactaccccatatcgtaggtgtgaaggaagcacagtctgtgctagatcagtacctattctcaactcaagagatactagcgaacactgttgagcaggatcagcaattcgacaggaggctgttagatgactttgtatctctaataggagacaagaagtaa

SEQ ID NO: 11 - DNA, 888 bp, Artificial Sequence
  Description of Artificial Sequence Synthetic polynucleotide
atgtcacaggatttgactctattcttggaacaatataaaaaggtcatcgacgaaagcctgtttaaagagatatcagagcgtaacatcgagccgagattaaaagagtctatgttatactctgtccaagcgggcggtaagcgtataaggcccatgttggtctttgccacccttcaagctctaaaagtcaaccctttactgggggttaaaactgcgacagccctggagatgattcatttcacctactttctaattcacgacgacctgcccgctatggacaatgatgactacaggaggggtaaatacacgaaccataaggtatttggagacgccactgcaatcctagcgggagacgcccttctaacgttggcatttagtattctggccgaagacgagaacttgtcatttgagaccagaatagcattaataaaccaaatctctttcagctctggagctgaggggatggtcggaggacaactagcagacatggaagcagaaaataaacaagtcactcttgaggaattatcttcaattcatgcaaggaagactggagagctactgatttttgcggtaacctcagccgctaagatagcagaggcggacccggaacagactaagagactaaggatatttgctgagaatattgggataggatttcagatttctgatgacatactagatgttattggcgacgagacaaaaatggggaaaaagacaggagtcgatgccttcctgaataagtctacctatcctggtttgttgaccttagacggcgcgaagagagctttaaacgagcatgtggcaatagctaaatccgctctgtcagggcatgatttcgatgacgaaatacttttaaaactggcagacctaattgcccttcgtgaaaattaa

SEQ ID NO: 12 - DNA, 1053 bp, Artificial Sequence
  Description of Artificial Sequence Synthetic polynucleotide
atgtcaaccggtgctattacggaacaactaagacgttacttacacgatagaagggcagaaacagcgtacataggtgacgattactcagggctgatagcagccttagaggagttcgtgctaaacgggggaaagagactgaggcccgccttcgcgtattggggttggcgtgctgttgcgaccgaggctccagatgaccaggcattattgttgttttcagccctggagcttctacacgcatgtgctcttgttcacgatgacgttattgacgacagtgcgacgagacgtggacgtccgacaacccacgtcaggtttgctagtctacatagggatagacaatggcagggctctccggaaagattcggaatgagtgcagcaatattattaggtgatctggccctagcgtgggcggatgacatcgtattaggggtggacctaacaccacaagccgccaggagggtaaggagagtatgggctaacataaggacagaagtcttaggcgggcagtatctggacattgtcgccgaggcatcagctgctgcttcaatcgcctccgccatgaacgtggacacttttaaaacggcatgttacacggtctctcgtcctttacaacttggggcagctgcggcggccgataggccagacgttcatgaccttttctctcagttcggaactgacctgggtgttgccttccagcttcgtgatgacgttctgggggtatttggtgatccagcggtaaccggtaaaccaagtggtgatgacttgagatccgggaaaagaacggttttgttagcagaagccgtagagctggctgagaagtctgatccactagcggccaaattacttcgtgacagcataggcgctcagttgtcagatgcggaggtagatcgtcttcgtgacgttatcgaatcagttggtgcattggctgctgccgagcaaaggatcgctactttgacacagagggcactggccaccctggcggctgcacctattaacactgcggcaaaagcaggcctgagtgaactagcgaaactagccacgaatcgttccgcttaa

SEQ ID NO: 13 - DNA, 981 bp, Artificial Sequence
  Description of Artificial Sequence Synthetic polynucleotide
atgtcaatccctgccgtaagtctgggcgatccccaatttacagcaaacgtgcatgatggcattgctaggatcaccgaactgattaacagtgaactttctcaagctgacgaggtaatgagagacacagttgcacatttggtagacgctggtggtactccatttagacctctattcaccgttcttgccgcgcagttgggtagcgatccagatgggtgggaagttacggtggcgggtgcagccatcgaactgatgcacctgggaactttgtgccatgatcgtgtggtagatgaatctgatatgtctaggaaaacgcctagtgacaatactaggtggaccaataactttgcaatattagctggtgactacagattcgctaccgcaagtcagcttgcaagtcgtcttgatcctgaggcttttgcggtcgtcgcggaggcgttcgcggagcttattaccggtcagatgcgtgcaacacgtggccccgcaagccacatagacacgatcgaacattaccttagggtggtccacgaaaagacaggctctctgattgcggcatctggacagcttggtgctgctttatccggcgcagcagaggaacagattagaagggtagctcgtttaggaaggatgataggagctgctttcgagatttcaagagatatcattgctatttcaggcgattctgctacgttatcaggcgcggacctgggacaggccgtccacacgttgccaatgctgtacgcactgcgtgaacaaaccccggacacgtctaggttaagggagctattagcgggtcctatccatgatgaccatgtcgcagaggcccttactctgctaaggtgcagtccgggtatagggaaggccaagaacgtggtggccgcttacgctgcccaagctagagaagagctgccatatctgccagacagacaaccgagacgtgcgttggctaccttgattgatcacgctatatccgcctgtgactaa

SEQ ID NO: 14 - DNA, 831 bp, Artificial Sequence
  Description of Artificial Sequence Synthetic polynucleotide
atgtcaaaattcaaggatttcagcaataggtatcttcccgaaatcaacaacgacctgagcaactatttcgcggacagggatgacgacatcttccgtatgataacatacgctttaaattcaacgggaaagagactaagaccgctactgacattggcaactttcgcggcggcgggaaatgttatcaacgattccaccattgaagctgcgactgccgtagaatttgttcatgcctactttctggtgcacgacgatctgcccgagatggatgacgacaccaaaagaaggaaccaatcttccacttggaagaagttcggcgtagggaacgccgtattggtgggggatggtttgctgaccgaggcgttcaaaaagatttctaacttatctttgcctgagtccataaggttaagattgatttacaatcttgctcttgccgccggtccggataacatggtgcgtggacagcaatacgacctattcagtcaagacaaggtcgagtccatagatgacctggagttcatccatttgatgaaaactggcgctttgatgacttacgcagctactgcaggtgggatactagccgggctgagcgatgataagctgagggcattgaacatatatggggctaatctgggaatagcgtttcagattaaggacgatctaagggacataaaacaggatgaagaggaaaataaaaagtcattcccccgtttaattggtgttcaaaaatcccagacagagctagaagaacacttaaagatttcagccaacgcgatcaaagaaatcccggactttcagaatacagtcctgctggacctacttgacagaatttaa

SEQ ID NO: 15 - DNA, 1125 bp, Artificial Sequence
  Description of Artificial Sequence Synthetic polynucleotide
atgtcagaagccgtcctgtccgccggtgcaggcgaatcaacgagaccatctcccagtgttcctccttttacggatactgttgaagacgctcttcgtgaatttttcgcgagtagagcagggacggtcgaaactgtaggtggcggttacgcggaagcagtcgctgccctagagagttttgtcctgagaggtggtaagagggttaggccgatgtttgtgtggacgggatggttgggggctggtggagacgcaaccgggcctgaggcgcctgccgctttgcgtgcggcgtccgcattggagttggttcaagcatgcgccttagttcatgacgacataattgacgcttccactacgagaagaggatttccaactgtccatgttgaatttgctgaccagcattcagctcatcattggtccggtggctcagctgaatttggtcgtgcagtggctatccttttgggggatttggcgttggcttgggcagatgacatgattagagaagcgggcctgagtcccgatgctcaggcgcgtatttccccagtttggtctgcaatgagaaccgaagttctgggaggtcaattccttgatataagctctgaagtgagaggcgacgaaactgtcgaggcagcattacgtgtagacaggtacaaaacagcggcttatactatcgagcgtcccttgcatctaggtgctgcgttggctggagcggatgatgcgttagtagcggcgtaccgtacctttggcactgatataggtatcgcgttccagctacgtgatgacctgttgggtgtctttggagaccccgagatcacagggaagccctccggcgatgatttgagagctggcaaaaggaccgttctgtttgctgaggcattgcaacgtgcagacgccagtgatcctgcggcggctgcacttctaagggaatccattgggacagacttgagcgatgcgcaggtagctacacttaggagcgtcattacggacttaggggctgtcgatgacgcagaaaggcgtatctctgaacttaccgacagtgctttatctgctttggacgggtctacagcgactgacgaaggtaagctgcgtttgagggaaatggccattgccgtaacgagaagagacgcctaa

SEQ ID NO: 16 - DNA, 903 bp, Artificial Sequence
  Description of Artificial Sequence Synthetic polynucleotide
atgtcagacttcccacaacagctagaagcgtgtgtcaaacaagctaaccaggctttgtcaagatttatagctccgctgcccttccagaatactccggtagtggagaccatgcagtacggggcattgttgggcgggaagaggctacgtccgtttctggtatacgcaaccggtcatatgtttggggtcagcacgaacacactggatgctcccgccgcagctgttgagtgtattcacgcatactttttgatccacgacgatttaccggcaatggatgacgacgacttgcgtagaggactgcctacttgtcatgttaaatttggcgaagccaatgccatactggcgggggacgcattgcagaccttggcgtttagcattctttccgacgctaatatgccggaggtttctgatcgtgacaggatctccatgatttctgagttggcttctgcgtccggcattgcaggaatgtgtggtggacaagcacttgatttagacgctgagggaaagcacgtaccgctggacgctctggaacgtatccatcgtcacaaaaccggcgcactgatacgtgctgctgttagactaggtgctctaagtgccggggacaagggaaggagagcccttcctgtcttagacaaatatgcagaaagtataggactagcttttcaagtacaggacgacatattagatgtggtcggcgatacggcaactttggggaaacgtcagggcgctgatcaacagctgggtaaatccacgtatccagcacttctaggtctggagcaggctcgcaagaaagcgagagatttaatcgacgacgcacgtcaggcacttaaacaattagcggagcaaagcctggacacatccgcgttagaggctttggctgactacataatacagaggaacaaataa

SEQ ID NO: 17 - DNA, 873 bp, Artificial Sequence
  Description of Artificial Sequence Synthetic polynucleotide
atgtcaaaagataagattaagtatattaaccaagccataaagcattactacgcacagacgcatgtgtctcaggacttagtggaagcagtgctttactctgtcgccgctggtggaaaaaggatacgtccccttttgctgcttgaaatcctgcaagggtttggtcttgtattaaccgaagcccattaccaggttgcagcaagtttagaaatgatacacactggttttctagtccatgacgaccttcccgctatggacaacgatgactacagacgtggccagctaactaaccacaagaaattcggtgaaactacggccatacttgctggggattcccttttcctagaccccttcggcttactagcgaaggccgatttgcgtgccgacatcaaaatcaagttggttgcggaactatctgacgcagctggaagctatggcatggtaggcggccagatgttggatattaagggagagcatgtgcagctgaatttagaccaacttgcccagatacacgctaacaagactggaaagctattaaccttcccatttgtggcagccggcatcattgcagagctatccgaaaaagcactggctaggctgcgtcaagtgggggaattagttggcttggcctttcaggtcagggatgacatcttagacgttacggcgagtttttctgaacttggcaagacccctcagaaagacatagaagctgataagtctacatatccctcattactgggtctggataaatcctacgctatactggaggacagtctgaaccaggcccaggcaattttccaaaagctggccctagaggaacagttcaacgcaacaggtattgagacgataattgaacgtctacgtctacacgcgtaa

SEQ ID NO: 18 - DNA, 888 bp, Artificial Sequence
  Description of Artificial Sequence Synthetic polynucleotide
atgtcacaagaggcgttaatcagctttcaacagaggaacaatcagcagttggagtggtggctttctcagctacctcaccagaaccagactttgatcgaggcgatgagatacgggctactattgggcggtaaaagggcaaggccctttctggtatacatcaccggacaaatgctgggctgtaaggccgaagatttagatacgcctgccagtgcggtcgaatgtattcatgcgtattctctgattcatgacgacttacctgctatggatgacgatgagttgagacgtggacaaccaacttgtcatataaagttcgatgaagccacagcaattttaactggggacgcattacaaacacttgcgtttagcatattggccgacggaccgctaaaccccaacgctgagtcaatgagaatcaacatggtaaaggtattagctcaggcttcaggtgccgcaggtatgtgtatgggccaagcgttggatttgcaggcggagaacaggttggtgaatcttcaagaacttgaggaaatacatagaaacaagacgggggctctgatgaaatgtgcgatacgtctaggcgcactagctgcgggagagaaggggcgtgaagtgttacccttactagacaagtacgccgacgcgataggattggcctttcaagttcaagatgatatcttggacattattagtgacaccgaaacattggggaagccgcagggttctgaccaggaacttaataagtccacatatccggctcttctaggacttgagggcgctattgaaaaagcaaataatttgttacaagaggcccttcaagcgctggatgcaattccatacaacaccgagcttctggaggaatttgccagatatgttatcgagcgtaaaaactaa

SEQ ID NO: 19 - DNA, 951 bp, Artificial Sequence
  Description of Artificial Sequence Synthetic polynucleotide
atgtcacacaagcccgttgatctgacggatacggcggccttcgagacccagttagacagatggaggggtagaatcggagaggccgttgctgaagcgatggcatttggcacgacggtgccagcaccgttacaggctgggatgtctcacgccgtcctggctgggggaaagaggtaccgtggaatgctagtgctggcgctgggttcagacttgggggtgcctgaggagcagttactaagcagcgctgtcgcgatagagaccatccacgcggcctcattggttgtagacgacctgccttgcatggacgacgcccgtcgtaggaggtcccaacccgccacgcacgtggcatttggcgaagcgacagctattttatctagtatcgcgctgattgctcgtgcgatggaggttgtcgcgagagacaggcaattaagtcctgcgtccagatcttcaatagttgacacactatctcacgcaatagggccacaggccttatgtggcgggcaatacgacgacttatatccgccctattacgcaacggaacaagatcttatacaccgttatcaaagaaagaccagcgcattatttgtggccgctttccgttgtcctgcattattagctgaggtagaccctgaaactctattaaggatagcgcgtgccggacaaaggctgggtgttgctttccagatattcgacgacctgttggatctgactggagatgcacacgccatagggaaagatgtcggacaggaccacggcaccgttacactggcaactttattaggaccagctagagcggcggaaagggctgccgatgagctagctgccgtacagaaagagcttcgtgaaactgtggggccgggtcgtgccttagacttgattagacgtatggccgcacgtatagctgggactggaaaaaaatctgcaggccgtgatgatctaaggcctcatgctgga

SEQ ID NO: 20 - DNA, 867 bp, Artificial Sequence
  Description of Artificial Sequence Synthetic polynucleotide
atgtcagcattcgagcagcgtattgaggcggctatggccgccgcgatagctagaggacaggggtcagaagccccgtcaaaattggccacagctctagattacgccgtcactccaggtggagcccgtattcgtccaaccttattattaagcgttgcgacgaggtgtggcgacagtagacctgcgctttccgatgccgccgctgtggctctagaattgatccactgcgcttcattggtacatgacgaccttccgtgttttgatgatgccgagataaggagagggaagccgactgtgcatagggcctactcagagcctctggctattctaacgggcgactctctgatagttatgggcttcgaggtcttggctggtgcggcggctgataggccacagagggcgttacagttagtaacggcactagcggtcaggacgggaatgccaatgggaatatgcgcagggcagggttgggaatctgaaagtcagatcaacttaagcgcttaccacagagctaaaactggtgcccttttcatagcagccacgcagatgggggctattgcagccggttatgaagcggaaccgtgggaagaactgggagcgaggattggagaggcattccaggtcgcagatgatctgagagatgctctgtgtgatgccgaaaccctaggcaagccagctgggcaagatgaaatacatgctaggcctagtgcagttagggaatatggtgtcgaaggtgcagcgaaaggcctgaaagacattttgggaggggccatagcgtctatccccagctgtcctgctgaggccatgctagccgagatggtccgtagatatgccgacaagattgtgcctgcccaggtggccgctagagtc

SEQ ID NO: 21 - DNA, 1086 bp, Artificial Sequence
  Description of Artificial Sequence Synthetic polynucleotide
atgtcagcccttactttacctgacgctcaaccccctacaggattgcttccccttgagcaagcgtggcttcagctggtccagacggaggtcgagacatctctggccgagctattcgaactgcccgatgaagcgggcctagacgtgaggtggacacaggcattaactcaagcacgtgcgtacaccctaagaccggcaaaaaggctacgtccagctttggtaatggcaggacactgcctggcacgtggctcagccgttgtcccgagtgggctttggaggttcgccgctggtttagaactactacatacatttttactgattcatgacgacgtagcagaccaagcagagctgagaaggggggctccacccctacatcgtatgttggctcccggaagagcaggagaagatttagccgttgtagtgggtgatcacttatttgccagggcacttgaagtgatgcttggatcaggacttacttgtgtcgctggtgtggtccagtattatctaggtgtatccggtcacactgcggcggggcaatacttagatcttgatctaggcagagccccgttagcggaggtaaccttgttccaaacattacgtgtcgctcacttaaaaacggccagatacggcttttgcgcacctttggtctgtgccgcaatgttaggaggcgcatccagcgggcttgtagaagagttagaacgtgtcggtagacatgttgggctggcttatcaactgagagatgatttacttggactatttggagatagcaacgtagcgggaaaggcggcagatggggactttcttcagggtaaacgtacctttccggttttagcagcctttgcccgtgcaacggaagcagaaagaacagaacttgaagccctgtgggctcttccggtagagcagaaggatgcagcagcactggccagggctagggcattggtcgagtcttgcggaggtagggcggcttgtgaaaggatggttgtaagggcgtccagggcggccaggcgttccctgcaaagtttacccaatcctaacggagtcagagaactgttagatgccctgattgcgaggctggcgcacagagcagct

SEQ ID NO: 22 - DNA, 1140 bp, Artificial Sequence
  Description of Artificial Sequence Synthetic polynucleotide
atgtcagaggccacattgtctgcagggactgccagggttggccagtcaagcacaaacactgcgccacatcctacatctcttgaacttccgggtgtgttcgagggtgccctgcgtgatttctttgattctagaagggaactggtaagcaatatcggaggcggttatgagaaggcagtttcaacactggaggcttttgtacttaggggaggtaaaagagttaggcccagttttgcttggacaggttggttaggcgcggggggagaccctaacgggagtggcgcggacgcagtcatcagagcgtgtgctgctctggagcttgttcaagcatgtgccctagtccacgatgatataatcgatgcttccactactcgtagaggctttcctactgttcatgttgaatttgaagaccagcatcgtggagaggaatggtctggggactccgcgcactttggggaggccgttgcaattttgttaggggatttagccctggcttgggcagatgatatgattagagaaagcgggatttctcccgatgcggcagctagggtaagtcctgtatggtctgcgatgcgtaccgaggtactgggaggacaatttcttgatatttccaacgaagcccgtggcgacgaaaccgtggaagcagctatgcgtgttaacagatacaaaacagccgcttacaccatagaacgtccgttacacttaggtgcggcgcttttcggcgcggacgctgagctaatcgatgcttatcgtacatttggcacggacatcgggatcgcgtttcaattaagggatgatttattgggagtttttggtgatccttctgtcacgggtaagccatctggcgacgacttgatagccggcaaaagaacagttttgtttgcaatggccttagctagagctgacgcggcggatccggctgccgccgagttacttagaaacggcatcggcacacagctaacggacaatgaagtggatacgttgagacaggtaataactgacctgggtgcggtaacggatgtcgagactcagattgatacgttagtcgaggcggcagccaacgcacttgacagttctacggcgacggccgaaagtaaggccaggttgaccgacatggcaatagctgcgaccaagagatcctat

SEQ ID NO: 23 - DNA, 975 bp, Artificial Sequence
  Description of Artificial Sequence Synthetic polynucleotide
atgtcaccggcaggagctctggcacctctagcagatttctttgctgcaggcgggaaaagacttaggccgactctatgcgtgctggggtggcatgcggcaggtggacagacgcctgcttcaagagaggtggtgcaagtagctgctgcgttggaaatgtttcacgcgttcgctcttatccacgatgatgtaatggatgacagcgacatccgtaggggagcgccaactttgcaccgtgcgctggcagggcagtacgctgatcacaggcctagggcattgaccgatagattgggtgccggcgccgccatattaattggcgacttggctctgtgctggtcagacgagctaatacatacggcaggtctgaggcatgatcaatttgcccgtattttgccggtgctagatatgatgaggaccgaggtcatgtacggccagtatttggatgtaaccgccacgggtcaacctaccgctgatattgggagggctcaaacgatcatcagatacaagaccgcaaagtacacgattgaaaggccgcttcagttaggtgcggaactagctggggcctctacagatgtgatagacgccttgtccgcctacgccgttcctttaggtgaagcgtttcaattaagagatgatctattaggcgcatttggagaccccgttgtaaccggaaaatcctcaacggaagaccttcgtgaggggaagccaacggtgcttgtaggcctagcattgagagacgcagctccagatcaagctgacgttcttaggaggctgcttgggaggagggacttaactgaagatcaagcaacccaaattagggctgttctaactggcactggagctagagcccaagtggagaacatgattgcacaacgtagagagcgtgttctggctctgctggacacgaacaccgtgcttgatgcgactgcagtcttccacttacgtcaattggccgattccgcaacaagaagaactagt

SEQ ID NO: 24 - DNA, 909 bp, Artificial Sequence
  Description of Artificial Sequence Synthetic polynucleotide
atgtcaacggtgtgcgccaaaaaacatgttcaccttactagagatgcagcggagcaacttctggcagatatagacaggaggttggatcaactgttaccagttgagggagagagggatgtcgtgggtgctgctatgcgtgaaggggcattagccccgggcaagcgtattagacccatgttgttgttactgacagcaagggacttgggatgtgcagtctcccacgacgggttattggatctggcctgcgcggtggagatggtacatgctgcgtctttaatactggatgacatgccctgcatggatgacgcaaaattgagaagggggcgtccaaccattcattctcactatggagagcacgtcgccattctggccgctgtggccctattgtcaaaggcctttggggtcatagcagacgcggatggccttacaccattggcgaaaaatagagctgtctcagagttaagcaatgcgatcggtatgcaggggctggtacaagggcagtttaaggatctgagtgaaggcgacaagccgcgttccgctgaggcaattctgatgacaaaccatttcaaaacctccacccttttctgcgcgagcatgcaaatggctagtattgttgccaatgcttccagcgaagctagagattgtttgcaccgtttcagcctggacttaggacaagcatttcaactgttggatgacttgacagacggcatgacggacacaggaaaggacagcaatcaggatgcaggaaagtctacgttagtgaatcttcttggaccgcgtgccgtggaagaacgtctacgtcagcatcttcaacttgcttcagagcatttgtccgcagcgtgtcagcacggtcatgctacccaacattttattcaagcttggttcgacaaaaaattggccgccgtcagc

SEQ ID NO: 25 - DNA, 984 bp, Artificial Sequence
  Description of Artificial Sequence Synthetic polynucleotide
atgtcagagctagataagtactttgatgaaataattaaaaatgtcaatgaggaaattgaaaaatacataaagggagaacccaaggaattgtacgacgcctcaatttacttgttaaaagcgggcgggaagaggttacgtccgttaattaccgttgcaagtagcgatcttttctctggtgaccgtaagagagcgtacaaggccgctgctgccgtcgagatcttacacaattttacgttgatacatgatgacataatggatgaagacacgttaagaaggggtatgccgacggtacacgttaagtggggcgtccctatggcaatactagctggagaccttttgcacgccaaggctttcgaggttcttagcgaagcgttagagggcttagatagcaggaggttctacatgggattgtccgaattttctaagtccgtaatcatcatagctgagggacaggcgatggacatggaatttgaaaataggcaggatgttacagaggaagagtaccttgaaatgatcaagaagaaaactgcacagttgttctcatgttccgcgtttcttggcgggcttgtaagcaacgcagaggacaaggatttggagctactgaaggagttcggcctgaatcttgggatcgcgttccaaataattgatgacattttgggtcttacggctgatgaaaaagaactgggaaaacccgtctactccgacatacgtgagggtaagaagacgattcttgtaatcaaagctctatccttagcttccgaggcggagcgtaaaataatcatcgaaggtcttggaagtaaagaccaggggaaaattacgaaagcggcggaggtcgtcaaaagtttatcactgaactatgcatatgaggtggccgagaaatactatcagaagtccatgaaagctctatccgccattggaggtaacgacattgctggcaaagcactgaagtatttagcggagtttaccattaagaggcgtaagtaa

SEQ ID NO: 26 - DNA, 1059 bp, Artificial Sequence
  Description of Artificial Sequence Synthetic polynucleotide
atgtcaacgcacgtacccgcgaacgcagtccccacaactaacggcttgtcaataatccctcccggtctgtcacttccgacaactttcgccccgttggtagaacgtatacaaactgttgctcacctagtagagacagcaatcgccgaggacttgtctgaagttacgcaacctgaactgcgtcaagcggttctacacctattcgatgggaaaggtaaaaggcttcgtccattcttggtgattacgaccgcagaggccgcgggcggcactcttgaagccgctttaccacccgctttggctgttgagtaccttcacaacctgagtctgattcacgacgatatgatggacgggtcccctgagcgtcacggtagaccaaccttacatactaggtttgggctaaacctgagtttgctggtaggggacttactttatgctaaagctgttgagcaagcctctcgtattaggcatcacgcgctaagaatggtgcacattctggggcaaactgccaagcagatgtgttacggtcaatttgacgacctgtactttgaaaggcgtttggatctaacaatagaggattatctaaggatggccgcaaggaaaacttctgccctttacagagcttcttgcatttttgggatgcttaccgcagacgcggatgaggccgaccttcaggcgatggctacctttggagagaacataggaaccgcattccagatctgggatgatgtattagacttgcaagccgatccgttacgtttaggcaagcccttaggccttgacattagggaaggcaaaaagacactaatcgttatccactttctacagcacgcttcccctgcggcgagaaggagattcctggaactgctaggtaaacgtgatttaaacggagaattgccggaggccatcgcgctgttggaggagacgggctcaatagcctttgcgcgtgacttggcgataaggtatctagtggacgcgaagcagcacctttccgtcttgcccgccggtccgcacaggaaattattagacatgtatgccgatttcatgctacagagaagacattaa

SEQ ID NO: 27 - DNA, 1050 bp, Artificial Sequence
  Description of Artificial Sequence Synthetic polynucleotide
atgtcaacctcagagacgaaggaggcgagagtgttggacgcaattagggagcgtagagatcttgtaaacgctgctattgatgaagaacttcctgtccaggaacccgagcgtctttacgaggccacgagatacatattagaggccggagggaagcgtctgaggcccacagtaacaactttagccgccgaggctgtaaccggaaccgagcctatgggggctgactttagggcctttcccagtttggacggggatgacgtagatgttatgagagctgcagtcgcaattgaagtcattcagagctttacacttattcatgatgacattatggatgaggatgacctacgtcgtggcgtcccagctgttcatgaggcctatgatgtctccacagctattctagctggcgacactctgtacagcaaggcctttgaatttatgacggaaactggcgcagacccgcagaacgggctggaagctatgcgtatgttagccagcacgtgtactgaaatctgcgaggggcaggcattagacgtttcctttgaaagcagggacgatatattacccgaagagtacctagagatggtggaactaaaaactgccgttctttatggtgcgtcagcggcaacacctgcgcttttgctgggagctgatgaagaggttgttgacgccttatacagatatggcatagatagcggacgtgcctttcagatacaagatgacgtgctggatctgactgttcccagcgaggagctggggaagcagagaggaagcgatttagtagaaggtaaggaaacattaatcacacttcatgccagacaacagggaatagatgtagatgggcttgttgaggcggatactcctgctgaagtaacggaagcggcaatcgaggaagcggtagccacattagctgaagcaggctccatagagtacgctagagagacagcggaagatttgactgcacgtagtaagggtcacttggaagttctgcctgaatccggttcccgttccctgctagaggacctagctgattacctaatagtaaggggctactaa

SEQ ID NO: 28 - DNA, 987 bp, Artificial Sequence
  Description of Artificial Sequence Synthetic polynucleotide
atgtcagaaacccttacccgttatttatcagagttcagaccgcttgttgataagaagataatggaggttcttgagggaagccctaaagaattatatgaagcggcccgtcatctgccctctaaaggagggaaaaggctgcgtccggctttagtattgttggtcaacaaagccctaggtggagaggtcgaaggtgcgttgcccgctgcagccgcggtcgaacttttacacaacttcacacttgtccacgatgacataatggatcgtgacgagttgcgtcgtggtgttccgactgtgcatgttttgtacggcgaatccatggcgattttggctggtgacttgttatatgcgaaagcatacgaggcgctgctacagtccccgcaaccacccgatcttgttaaggaaatgaccgaagtgttaacttggtctgccgtgacagttgccgagggtcaagccatggatatggaatttgaaaagcgttgggacgtgaccgaggaagaatatttggagatgatagaaaagaaaacaggggcactttttggagcttccgcagctctgggggcgctgaccgcaaataagcgtgaggtcaaagatctgatgaaagagttcgggctaattttagggaaggctttccagataaaggacgatgtgctttcccttttaggtgatgaaaaagttaccggaaaaccaaagtataatgatcttagggaggggaagaaaaccatcctggtgatttatgcgttgagaaatttaccccgtgatgaagcagaaagagtaaagtcagtgcttggcagagaaacctcctacgaagcgttagaagaagttgcagaactaattaagagaagtggtgctctggattacgctatgaaactggctgaagagttcgagaaaagagcgtacgagatattagaaactgtcaggtttgaagacgaagaggcgatgagggccctaaaagagctggtcgatttcgcagttaagagggaatattaa

SEQ ID NO: 29 - DNA, 1074 bp, Artificial Sequence
  Description of Artificial Sequence Synthetic polynucleotide
atgtcagggaaacaattcaacctgctgagggaaaaatatcttccgcaaatcgaaagggagattaagaaatttttcgaggagaaaatcagcacacagaaagacgaggtcattgtcagatactacgaggaactgtcttcatacgtacttagaggagggaaaaggttcaggccgcttgcccttatctcatcttattatgggagcggctcaaagcatgaaggtaacattattagggcatcaataagcgttgagcttctacacaacagctctttgatacacgacgatataatggacgaaagtccaaagagaaggggtggtccaagttttcattatctgatggcaaattggagtaggctatcccccagaacgcctccaccaaggaaccccggaatctctctaggcattctgggcggggactccctaatcgagctaggcttagaggctctacttgagagtggatttccaaacgagatcattgttaaggccgctagtgaatattccgttgcatatagaaagctgattgaagggcagctacttgacttatatctgtctacagttaccatgcctactgaagaggaagtactgcgtatgctttctctaaagaccgggactctatttagcgcatccttagttatgggtggcatgttagccggtgcatcagaggatatgttacactttctaaggtcttttggtcagagagttggggtagcattccagttacaagatgacattctgggactttacggggacgaagccgtcatcgggaaaccagccgattctgacataaaagaaggtaaacgtacgctattggtggtgaaagcctgggaactgtccgatgaggccaccaggaagaagctactttccatacttggcaaccccaatatcagtgctgcagatctaaactacgtccgtgaggtagttaaagagctaggagccttagactacactcgtaagaccgcccttaatctactgaaagagaatgagaaagatattgagttcaacaaacacttgttcgaggaatcatttgtagagtttttgaaggagctgaacgaaattgtaatagcgaggtcattctaa

SEQ ID NO: 30 - DNA, 1041 bp, Artificial Sequence
  Description of Artificial Sequence Synthetic polynucleotide
atgtcatcaaatatcaacgaagatgtcgggaaagttcttggtcagtatagtaaagacatacacaaggaaatcggaaacacactgagcaacattggacccgaggatctaagagaagcgagtatttacctgaccgaggcaggtggtaaaatgctacgtcccgctctgaccgtgcttatctgtgaagcagtaggcggcacgttcagcagctgtataaaagcagctgcagcgatagaattgatccatacattcagcttaattcacgacgacataatggataaggacgatatgagaagaggtaagccgtcagtccacaaggtgtggggcgagccggttgccatacttgcgggtgacaccttattttctaaggcttatgagttggtgatcaacagtaagaatgaaatagattcttctaaccctgaagagtgtctgaacagggtgaaccgtaccttgagcaccgttgcggacgcgtgtgttaaaatatgtgaagggcaggcacaggatatgggcttcgaaggtaatttcgatgtatctgaagaggagtatatggaaatgatcttcaaaaagaccgctgctctgatagcggcagcaaccgaatccggggccataatgggtggtgcgaacgaaaagattgtgagcgatatgtatgactatggtaaattaataggtctagcgttccaaatacaagacgactatctggaccttgtcagcgacgaagatagcttaggtaaacccgtcggttccgacatcgcagaaggaaagatgacaatcattgtagttaatgcattaaacagggcgaacccagaggacaaaaagcgtatcttggaaattcttcgtatgggcaatgagtcaggtaactgcgaccaagtctatgtggatgaagcaatatctctattcgagaaatacgggagtatacaatacgcccagaatattgctttggccaacgtcaaaaaggccaagcaactgcttgaaatactaccggaatccgaggctaagcatactctttccctagttgccgactttgttttatatagacaaaactaa

SEQ ID NO: 31 - DNA, 972 bp, Artificial Sequence
  Description of Artificial Sequence Synthetic polynucleotide
atgtcatcagatttgaagacctacctagagaagacggcggaacaggtcgatatcgcattggaaagaaactttggtgacgttttcggagacctttataaggcttcagcgcacctactattagcagggggaaagcgtttacgtcccgccgtactattgctggcggctaatgcggttaaaccaggacgtgcagacgacctaattacggctgccatagccgttgaaatgacacacacgttttacttgatacatgacgatataatggacggtgatgttaccagaaggggtgttcccacggttcatactaaatgggacgaaccaacggccatactagcaggggacgtattgtacgccaagtcatttgagtacatcacgcacgctttagcggaagatcgtgctcgtgtgaaggctgttacactattagcccgtacttgcacggaaatctgcgaaggtcaacaccaagacatggcctttgagcaaaaaggcgctgaagtagaggaagcggactacattgagatggctggtaagaaaacaggtgctctatatgccgccgctgccgctatcggtggaactcttgccggtggaaacgcaatgcaggtggacgcactttaccaatatgggatgaatgcgggaattgcttttcagatccaagatgatctgatagaccttctagcgcctccagaaacctccggaaaggacagggcatctgaccttagggaggggaagcaaacattgatcgccattatagccagggagaaagacctagatctttcaaagtacagacacacgctgacaacgacagagattgacgctgcaatcgcagaactggaaggtgcaggtgtagttgacgaggttaggagggctgcggaagaaagagtggcgaccgctaagagagctttatccgtgctgccggagagcatggagaggacctacctagaggagatcgctgattacttcctgaccagatcattctaa

SEQ ID NO: 32 - DNA, 969 bp, Artificial Sequence
  Description of Artificial Sequence Synthetic polynucleotide
atgtcagatcttatcgacgagctgaaaaagcgttcaacacttgtagacgagtctatacaggaatttttgcccatcgatcaccctgaggagctgtaccgtgcaacgaggtatttacccgacgctggtggtaaacgtctgagaccagctgtgcttatgttaagcgcagaagcagtgggcggcgacagtgactccgtattgcctgctgcggttgcacttgaactaatccacaacttcaccttgattcacgatgacatcatggatagagacgacataaggagggggatgcccgcccttcacgtaaagtggggaactgcaggtgccatcttggccggtgacacactttactcaagggccttcgagatcatatcaaaaatggatgctgatcctcaaaaattgctgaagtgcgttgctttgctaagcagaacctgcactaagatctgtgagggacagtggttggatgtggactttgagaaaagagatatcgttgatgtggatgaatacctagaaatgattgaaaataagacgtcagtcttgtatggtgctgctgctaaggttggagcgattctgggaggcgcgagtgatgaggttgctgatgctatgtatgagttcggtaggctaacgggcattagtttccagatccatgatgacgttatagacctggttacccctgaggagattcttggtaagagtagaggatctgacctgaaagaagggaaaaagacattaattgcacttcacgctctaaacaatggtgtagaattggaatgttttggtaaagcagacgccacgcaagacgaaataaacaatgctgtcgctaaattggaagagagtggtactctggcttatgtccgtgagatggctgacaactacttagaagacgggaagagtaagctggacttattagaagatagtcccgcgaaagaaaccttaatcgagatcgcagattacatggttagtagagaatactaa

SEQ ID NO: 33 - DNA, 972 bp, Artificial Sequence
  Description of Artificial Sequence Synthetic polynucleotide
atgtcagatcttattgaagaaattaagaaacgttcatctcacgtagataaaggtatagaggagtacttgccaatcgataagccctatgaattatataaagctgcaagatatctaccagacgccggaggaaagcgtctaagaccggcaactgtaatacttgctgccgaggccgtcgggagcgacctagagactgtacttcctgctgcagtagcggtggaacttgttcataatttttatttggtccacgatgatatcatggatcgtgatgatataagaaggggtatgcctgccgttcacgtgaaatggggcgaggcaggcgccattctggctggcgatacgctgtattcaaaagcctttgagatattaacccacgctcccgcagaggccccggagagaaacctaaagtgtattgatatcttatcaaaagcgtgtcgtgatatttgcgaggggcaatggatggatgtagagtttgagaacagggatgacgtaactaaagaggaatatctggaaatgatcgagaagaagactggagttttatacgccgcgtctatgcagataggtgcaatcctgggtggcgcgcctgaagaggtgagtgacgctttttacgagtgcggcagactaatcggcatagcatttcaaatttatgatgacgtaattgacatgactacaccagaagaggttttagggaaggttcgtggttcagaccttatggagggtaagaaaacacttatagcaatacatgccttgaacaagggtgtcgaattaaagattttcggtaagggtgaagcgaccactgaggaaattaatgaagcagttcaccagcttgaagaagctggcagtatagattatgttagagatttagcccttgactatatagcaagaggaaaggaattgttaaacgtagttgaagactccgagtccaagaccatacttaaagctatagcagactatatgataactaggtcttattaa

SEQ ID NO: 34 - DNA, 966 bp, Artificial Sequence
  Description of Artificial Sequence Synthetic polynucleotide
atgtcaattgaggaaatattacaaaagaaggccaaattggtagacgaaagcatacctaagtttcttcctataacgccgccggacgaactgtataaagccatgaggcacctgttagatgctggcgggaagagactaaggccttcagctttactacttgccagtgaggccgtaggcggtaaacccgatgatgtcctgcctgcggctgttgcggttgagttagtccacaacttcacattgatacatgatgacatcatggacgaggcggatctgagaagaggtcttgcaacagtacacaagaaatggggagtaccaagagctataattgcgggagacgcactttactctaaggcatttgagattctatcttgcacaaagagcgaaccccagaggctggttgaaagtcttgagctactgagtaaaacatgcacggacatctgcgagggccagtggatggatatgaatttccagacaagaaaagatgtaaccgaagaagaatacatgcgtatggttgaaaagaagaccgcggtgttgtttgccactgcactgaaattgggggcggtcctgagcggtgccaatagggaacacgtaagagccctatgggacttcggcaggctaactggagtcggttttcaaatatacgatgatgtgatagatctaataacaccagaagagatactgggtaaagcgcaaggcggcgacataatagagggtaagaggaccttaattatcatccacgctctaagtaaagggatttctattgacgccttaggcaagtgcaacgctactaggtctgagatcagtgcagcattaaccacgctaaaggaatccggatctattgattatgcaatgaacaaagcactaagtttcgtcgatgaaggcaaagcagctctagcgatgctgcctgaatcagaggcgaaaaacattctaactcgtttagccgactatatgattgagcgtaaatattaa

SEQ ID NO: 35 - DNA, 981 bp, Artificial Sequence
  Description of Artificial Sequence Synthetic polynucleotide
atgtcagaagccgatatgagcgacttatcagcctaccttaaatctgtggcacagcaaatagacggtatgatcgagaaaaactttacccatgcagggggagagttggacagagcctctgcacacctattgagcgcaggagggaaacgtctgaggcccgccgtggtcatgctgagtgcagacgcgatcagacatggctcaagtaaggatgtaatgcccgccgcccttgctttggaggtcacccatacattttacctaatacatgatgatatcatggatggagatagtctgagacgtggagttccaactgttcatacgaagtgggatatgccaacaggtattcttgccggagacgtcctttatgctagggcattcgagttcatttgccagagtaaggctgatgaaggccctaaagtgcaagccgtagctttgttggcaagggcttgcgccgatatatgcgaaggtcaacaccaggacatgtcattcgaacatagggcagatgtaactgaagaagaatacatggctatggtggctaaaaagacaggcgtattgtacgcagcggcggctgctatcggcggaacactggcgggagggaacccggaacagatcagggctttgtaccagtttgggttaaatacaggaatcgcctttcaaatacaagatgatctaattgatcttctgacccccactgagaagagtggaaaagaccagggtagcgacctgagggagggaaagcaaactctggtcatgatcattgcaaggcaaaagggtgtggatctattgaaatatagacacgaactttctcctgctgacattaaagcggcaatccaggaattaactgatgcgggtgtcattgacgcagttaagaagaaggcggctgatctagtggcagattccaataggttgcttatggtccttccgcccactaaggagagacagttgattatggacgtaggggagttcttcgttacgaggtctttttaa

SEQ ID NO: 36 - DNA, 966 bp, Artificial Sequence
  Description of Artificial Sequence Synthetic polynucleotide
atgtcagagttgattgaatatctggaaaaggtagggaaccaagtcgatcgtttaatcgataggtattttggagatcctgtgggtgaactaaacaaagcgagtgcgcacctgcttactgccggtggcaagcgtcttcgtcccgcggtaatgatgctggctgcagacgctgtaaggaagggctcttctgacgacttgatgccggctgctatcgctttagaattgactcattcattttacttaatccatgacgatataatggacggcgacgaggttagacgtggagtcccaactgttaacaaaaagtgggacgagccaaccgccattttagcgggggatgtgctttacgcgagggcttttgcattcatatgtcaagcccttgcaatggacgctgctaaactgagagcagtttccatgttggcggttacgtgtgaggaaatttgtgctggacagcatcttgacatggccttcgaagatagagatgatgtttcagaagaggaatatcttgaaatggtcgggaagaaaactggcgctctttatgcagcatcaactgctatgggtggagtccttgcgggtggttcccagccgcaagtagatgcgctttaccgttacggcatgaacatcggcgttgcgtttcaaattcaggatgacctgattgatcttctggcgtctcccgaaaggtcaggtaaagatagagctagtgacatacgtgaaggaaagcaaacactgattaacataaaggcgagggagcacggttttgacttagccccatacagaagacgtttagacgatgctgagatagacgacttaattcagcaattaacagataatggcgttattggcgaagtaaaagccactgcggaaggactggtcacttctgctggtaagatccttgctattttgaaaccatcagacgaaaaggacttattgataagtataggtacctttttcgttgaacgtggctactaa

SEQ ID NO: 37 - DNA, 1005 bp, Artificial Sequence
  Description of Artificial Sequence Synthetic polynucleotide
atgtcaaaagatacgacgaagattgaagtggagaactacattaataaagtgaataaccatctaatttcatttcttagtgggaagccacttcaattatatcaagcaagcacgcattacctgaagtcaggcggaaagagattaagaccgataatggttatcaaatcctgtgaaatgttcggtgggacacaacaggatgcactacctgcggcagcagccgtcgagtttattcacaacttctccctagtgcacgatgatattatggataacgatgaccttcgtcacggtattccaactgtgcataagagctttggattaccgcttgcgatccttagtggtgacattttattttccaaggcttttcaaatacttagtataaccaacgtaaactcaattaaagattccagccttctatcaatgataaggaggttgtccctagcctgtgtagatatctgcgaaggtcaggctaaagatatacagttcagcgaatgtgagacttttccatcagaagaggagtatcttgaaatgatctcaaagaagacggcagctctttttaacgtgtcctgttcactaggcgcgttatcaagtaggaatgccacagaaaaagacgtcaataatatgagtgactttggcaaaaattccggtattgcgttccagttgattgacgacctgataggaatcgcgggacactcaaaagagacgggcaaagccgtaggcaatgatattcgtgaagggaaaaagacatatccgatcctgttatccatcaagaaggcgagcgagttggagagggcgcacattctaaaagtgttcggcaaagggcagtgcgataacatgagcttaaagaaggcaatagacgtcatctctagcttgcagatagagaagatcgtcaggaaatccgcgatggcatatatcgaaaaggcgatggaggccctggttaattacgaggattctgaaccgaagaagatattacaggagttgtcatcttacatagtagagcgttctaaataa

SEQ ID NO: 38 - DNA, 999 bp, Artificial Sequence
  Description of Artificial Sequence Synthetic polynucleotide
atgtcactgcaagactactttaacgaagtaatcaatcaggtgaacaaaaccattgagaaatacctaagtaacgccccgagcggaacgagtagcttatacgaggcctcaaaacatttattttctgctggaggaaagagactgagacctctaatcttggtaagctcctgtgactttctgggtggcgaccgttcacgtgccatcctggcaggatcagccatcgaaactctacatacattcacattgatccacgatgacatcatggaccatgattttctgagaaggggcttgcctactgtacatgtcaaatggggtgaatctatggctatcttggctggcgatctacttcacgctaaagcatttgaaatgctaaatgactcactagaaggggtgaatgagacgctacactacgaagtgatgaagacatttattaattctatcgtggtagtgagtgaagggcaggccatggacatgcagtttgaggggcgtaatgacgtgacagaggaggactacctggaaatggtaaagaagaagactgcttatctaatcgccactagctctaaaattggttcattaattggcggtgcgggcccagatgtcgccgacaaattctttcacttcgggatttatcttggcatagccttccagattgttgatgacatcattggcataacatcagacgaggctgagctgggcaagccgttattttctgacataagggaaggaaaaagaacacttctggtaatcaggacgttaaaggaagccgagtcacgtgagcttgaagttcttaaacaagttttgggcaataagaatgccagtaccgaccaactgaaagaggcctcccaaatcgtcaagaagcactctttggagtacgcatacagtttagctgaggagtatagatccagagctatctcatcacttgatggcatacagccgcgtaatcaagaggcttatgaggccctgaagttcgtgagcgaatttacgttaaagaggaaaaagtaa

SEQ ID NO: 39 - DNA, 987 bp, Artificial Sequence
  Description of Artificial Sequence Synthetic polynucleotide
atgtcatcttttaattcaatctccaaaacagcaaagaaggtgaactcatttttattgtctagcttacacggaaaccctgaggagatttacaaagctgcgagctacttgattgaatacggcggaaaaaggttacgtccgtacatggtaataaaatcttgtgaaatacttggaggcacaatcaagcaggcattaccatctgcagccgcaatcgagatggtccataactttaccctaatacacgacgacattatggacaatgacgaaattagacacggcgtgagcacgacccataagaaattcggcatccccgtagggattcttgcgggggatgtgctgttttccaaagcgttcgagaccatttcacatggagatcctaagatgcccaaagacgtcagattagccttagtgtcaaaccttgccaaagcgtgtactgatgtgtgcgaaggccaagctcttgacattatgatggccaaatcacagaagattcctactgaggagcagtatattatgatgatcgaaaagaagacaagtgcattgttcgcagcggcgtgtgcgatgggcgcaattagtgcaaacacaaagacgagggacgtcacaaacttatctagctttggcaaaaacctgggagttgcgtttcaaatcgtagacgatttgattggaattattggtgattctaagataaccaaaaagccggtcgggaatgatttaagagagggcaaaaagagtctgccaattttgttggccattaacaaagtctctggtaagaagaaggaaattatcctgaatgcctttggtaattccgcgatatcaaagaaagagcttgagaacgcagtgaggattattagctccatggggatagaaacggctgttagaaagaaggccatacaatactccaatgccgccaaaaagagcttgagcaactataaagggagtgctaaaaatgagctgctttccttactagacttcgtggtcgagagaagccagtaa

SEQ ID NO: 40 - DNA, 1035 bp, Artificial Sequence
  Description of Artificial Sequence Synthetic polynucleotide
atgtcaggcaaatatgatgagttatttgcccaagtgaaggctaaggcgaaagacgtggacgccgtaatttttgagctaatacccgaaaaggagcccaagacgttgtacgaagctgcgagacattatcctttagctggaggcaaaagggttcgtccctttgttgtgttgagggcagccgaggcggttggtggcgaccccgaaaaggctctgtacccggctgccgcagtagaatttattcataattattctctggttcatgatgacatcatggatatggacgaactaagacgtggcaggcccactgtgcataagttatggggcgtcaacatggccatcctagctggcgacttgttattcagtaaagcattcgaggccgttgcaagagctgaagtaagccctgaaaagaaggctaggatattagacgttttggtcaagacctcaaatgaattgtgtgagggtcaggccctggacattgagtttgaaaccagggatgaggtaacagttgatgaatatcttaaaatgatttctggaaagacaggtgcgttgttcaatgggtctgccaccatcggagccatcgtaggaacggacaacgagaagtacattcaagcactgagtaagtgggggaggaatgtcggtatcgcctttcaaatctgggacgacgttcttgatcttatcgcagatgaagaaaaactagggaaacccgttggcagtgacataagaaaagggaagaagacgttaattgtgagccactttttccagcacgcgaatgaagaggacaaagccgaatttttgaaggtatttggtaagtacgcgggggatgctaagggagacgcgcttatacatgatgaaaaggtcaaagaggaagtggccaaggcgatcgaacttcttaaaaagtatggatctatcgattatgccgctaattacgctaagaacttagttagagaggctaacgaggcgctaaaggtgctaccggagagcgaggcgaggaaggaccttgaattactagccgaatttttagttgaaagagaattttaa

SEQ ID NO: 41 - DNA, 987 bp, Artificial Sequence
  Description of Artificial Sequence Synthetic polynucleotide
atgtcagatattataagcaggttctccgaaaagatcgacgccgttaattctgcaatagacaagttcctaaggatacgtgaacctaaaagactgtactctgcgacgagacaccttccacttgcaggaggcaagaggctacgtcctattctggcaatgttatcaacagaagccgtaggcgaggactggaagaaaacaataccctttgcggtgtccttagaacttcttcataatttcactctggtgcacgatgatataatggaccgttccgatcttagaagaggaatcgaaacagttcacgtgaagttcggcgaacctactgctatacttgcgggagatatacttttcgctaagtccttcgaggtgctttacgaattagatattgacgacgcaatcttcaaaactgttaatagattactgatagattgtattgaggaaatatgcgatggacagcagatcgatatggaatttgagtcacgtaaatacgtcagcgaagaggaatatcttgagatgattgaaaagaaaacaagcgcactgtttagttgcgcgacaacgggtggtgccattatcggggacgggaataaccgtgaagtcgattctctttccttgtacgggcgtttcttcggtctagctttccagatttgggacgactacttggatatcgcgggggaggagggggaatttgggaagaagataggaaacgacattaggtgtggcaagaagaccctaatgatcgttcacgcgactaagaatgctgatgggagagagaaggaaacgatcttctctattcttggaaagaaggatgcaacggatgaggaaattaacgaggtaatggagatcttaacaaagtctggaagcattgactacgcgaagaaaaaggcgttacactttgccgaaaaagcaaaagaacaacttagggtgttaccagattcaagggccaagagggatttgattgaattagtcgatttcgccattagcagagaacgttaa

SEQ ID NO: 42 - DNA, 960 bp, Artificial Sequence
  Description of Artificial Sequence Synthetic polynucleotide
atgtcacttattgaccactatattatggattttatgtcaattacaccagatcgtctgagtggtgcttcccttcatttgattaaagcgggtggaaaaaggctaaggcctttgattaccttgctaacagcgaggatgcttggaggtctggaagcagaagcgagggcgataccgctggcggcatccattgaaacggcccataccttctccttgattcacgatgacattatggatagagatgaggtgcgtagaggcgtaccaacaacgcacgttgtctatggagatgactgggcgattctggcaggggatacccttcatgcagctgcatttaaaatgatcgccgattccagggagtggggtatgagtcacgaacaggcctatagggcttttaaggtattatcagaggcggcaatacagatatcaagaggtcaggcatacgacatgttgttcgaagagacttgggatgtagatgtcgctgactacctgaacatggtaaggctgaagacgggagctttgatagaagcggcagccaggatcggcgctgtagcagcaggggctggatcagagattgagaaaatgatgggcgaagttgggatgaacgcgggtatagcgttccagattcgtgatgacattcttggcgtcatcggagatcccaaagtcactggaaagcccgtctacaacgaccttaggagaggcaaaaagaccctgttggtaatctatgctgtaaaaaaagcgggtaggcgtgagattgttgaccttataggccctaaggcgtcagaggacgatttaaagagggcagctagtatcattgttgacagtggtgctctagattacgcggaatcaagagctaggttttacgtggagagagctagggatatattgtctcgtgtccccgcagtagacgcggaatccaaagaactgcttaatttgttactggattacatagtggaacgtgtcaaataa

SEQ ID NO: 43 - DNA, 963 bp, Artificial Sequence
  Description of Artificial Sequence Synthetic polynucleotide
atgtcaatctcagaaataattaaggatagagcgaagctagtgaatgagaagatcgaagaactgctaaaggagcaggagccggaggggttatatcgtgcagcgcgtcattacttgaaggctggcgggaagagattgagacccgtcataaccctgttgtcagcggaagccttgggtgaggactacaggaaggcgatccacgcagcgattgctattgagactgttcacaacttcaccctagtccatgatgatattatggatgaggatgaaatgagaaggggcgtgaagactgttcacacattgtttgggattcccacagctatcttagctggagacacactatatgccgaagcattcgaaatcttaagcatgtctgatgcgccgccagaaaacatcgttagggccgtctctaaacttgcgagagtttgtgttgagatttgcgagggccaattcatggacatgtccttcgaagaacgtgacagtgtcggcgagagtgagtacttggagatggtccgtaagaagactggcgtgcttataggtataagtgcaagtatccccgcagtactgttcggtaaggatgaatctgtggaaaaagccttatggaattatgggatttactcagggattgggttccagatccacgatgacctgctggatatttcagggaaaggtaaaataggcaaggactggggttccgatatactagagggcaaaaagacactaatagtaattaaggccttcgaagaaggaatcgaactagagacgtttggaaagggcagggctagtgaagaggagttagagagggatattaaaaagttattcgactgcggagctgtcgactacgctagggaaagggccagagaatatattgagatggcgaaaaaaaacttagaggtcatagatgaaagcccatctagaaattacctggttgagttagcagactacctgattgaaagggatcattaa

SEQ ID NO: 44 - DNA, 1044 bp, Artificial Sequence
  Description of Artificial Sequence Synthetic polynucleotide
atgtcatccgaacgtcatcaacaggtagaggacgcaatcgtagcacgtcgtgatagggttaatgacgcactacctgaagatctgccagtgaagaagcctgaccacctatacgaagctagtaggtatctgcttgatgccggggggaaaaggttgaggcctacagttctgctgctggtggcagagtcccttcttgatgtggatcctcttacggcagactatcgtgattttcccaccctagggggcggccaggcagacatgatgtctgcagctcttgccatagaggtgattcaaacttttactctaatacatgatgatattatggacgacgacgctttaaggcgtggggttcccgcagttcataaagaatacgacttgagcacagcaatcttagccggagatacattatattccaaggcttttgagttcttgctagggacaggtgcagcgcacgaaagaacggtcgaggcaaacaagagattagcgacgacctgcacacgtatttgtgaggggcagagcttggacattgaatttgaacagcgtgacgttgtcacaccggaagagtacctagagatggtggagctgaaaactgcagtattatatggagcggcggctagcataccagctacattattaggagcggatgccgagaccgtcgacgcgttgtataactacggacttgatgttggaagagcttttcaaatacaagacgatttgttagatttaacaacaccatccgaaaaattgggtaagcaaagagggtccgatctggtcgaaaacaaacaaacgcttgttactctgcatgccagacaacaaggagtggatgtcggcgacctaattgataccgattctgtagaggctgtaagtgaagcagaaattgatgctgcagtcgagagactgagggaggtcggttctattgaatatgcacgtcaaactgggcaagaccttatcgcgagcggcaaacaaaacttagaggtattaccggacaatgaaagcaggtccctattagaaggtatcgcaaactacttagtagaaagagactattaa

SEQ ID NO: 45 - DNA, 978 bp, Artificial Sequence
  Description of Artificial Sequence Synthetic polynucleotide
atgtcaatgcttatgacgctggtcgatgagatcaaaaatcgttccagccatgtagatgcagctatagatgaattgcttcccgtgacgcgtcctgaagagctgtataaggcttcaaggtatcttgtggacgctggaggaaagcgtctaaggccggccgtcctaattctggccgcggaggcagtcgggtccaatcttaggtccgtcctacccgccgccgttgcggtagaacttgttcacaactttacgctaatacatgacgacattatggatagagatgacattcgtcgtggaatgcccgccgttcatgttaagtggggtgaagcaggcgcgattctagcgggggataccctatattcaaaagcgtttgagattctatcaaaggtggaaaacgagcctgtaagagtactgaagtgcatggacgttttatccaagacttgcacagagatttgtgaaggtcaatggctggacatggactttgagactaggaaaaaggttaccgagagcgaatatctggagatggtcgagaagaagacctctgtactgtatgcggcggccgccaaaattggagcgttgcttggaggggcctccgatgaggtggcagaggccctaagtgaatatggaaggcttattggaattgggttccagatgtacgatgatgtcttagacatgaccgctccagaggaggtgttaggaaaggtaagggggtctgacttgatggaaggtaagtatactttaatcgtgatcaatgccttcgagaagggcgttaagttggacatatttgggaagggcgaagcgaccctagaagagaccgaagccgccgtaagaacccttacagaatgtggaagcctagattatgtaaagaatctagcgattagttacatcgaggaaggtaaggaaaagttagacgtgcttagagattgtccagaaaagacacttctgttgcagatcgcagattatatgatctcccgtgagtactaa

SEQ ID NO: 46 - DNA, 981 bp, Artificial Sequence
  Description of Artificial Sequence Synthetic polynucleotide
atgtcaaccgaggtcctggatatactgagaaagtactcagaagtcgccgacaaaagaataatggagtgtatttctgacatcacaccagatactttgcttaaggcgagcgaacacctaataacggcgggcgggaagaaaatacgtccctccctggccctgctatcatgtgaggcagtgggggggaaccctgaagacgccgctggcgtagccgcagccatcgagcttatacatacatttagtttgattcacgacgacataatggatgatgacgagatgagaaggggcgaaccctctgtgcatgtcatttggggggaaccaatggctatcttggcgggagatgttcttttctctaaggcctttgaagcggttatcaggaacggcgattctgagcgtgtgaaagacgcactggctgtagtagtcgacagctgcgtcaagatatgtgaagggcaggcgctggatatggggttcgaggaaagactagacgtgacggaagatgaatacatggagatgatctataaaaaaaccgcagcactgattgctgctgcaactaaagccggggccatcatggggggtgcgtccgaacgtgaggtggaagctcttgaggactatggtaaattcatcggtttggcctttcagatccatgatgattaccttgacgttgtctcagacgaggagagcctggggaaaccggtcgggagtgacatagcagaaggtaaaatgactttaatggtcgtaaaagcgttggaggaggcttcagaggaggatagggaacgtctaatttccatccttggttctggagatgaaggcagcgttgccgaggccatcgaaatatttgaaaggtacggggctacgcagtatgcacacgaggttgctttagactacgtcaggatggcaaaagaacgtcttgaaatcctagaagactctgacgcgcgtgacgccttgatgcgtatcgcggatttcgtgttagagagggagcactaa

SEQ ID NO: 47 - protein, 367 aa, Artificial Sequence
  Description of Artificial Sequence Synthetic polypeptide
MSSDSSSIGAIETRIRELVHDYVGVNGTDAPITPALRPMFHTVVDQALASSEGGKRLRALLTLDAYDVLAGAPDSTQSRSVRTKVLDFACAIEVFQTAALVHDDLIDDSDLRRGKPSAHCALTSFAGARSIGRGLGLMLGDMLATACTLIMEDASTGMVEHRRLVEAFLSMQHDVEVGQVLDLAIERMPLDDPQALAEASLDVFRWKTASYTTIAPLMLAFLASGMTSEAANLHCHAIGLPLGQAFQLADDLLDVTGSSRSTGKPVGGDIREGKRTVLLADAMMLGTAAQRVQLQQLYEQPFRSDAQVHETIALFHDTGAIEHSHERIAKLWSQTQESIEAMGLTAAQSQSLRKACERFLPDFTAER

SEQ ID NO: 48 - protein, 361 aa, Artificial Sequence
  Description of Artificial Sequence Synthetic polypeptide
MSCTTANNREIIEPRIIQLVRELTAAPATDEVADALKPVMEQVVDQAASSSQGGKRLRALLALDAFDILAGDVTPDRRDAMIDLACAIEVFQTAALVHDDIIDESDLRRGKPSAHHALEQAVHSGAIGRGLGLMLGDILATACIEITRRSASRLPNTDALNEAFLTMQREVEIGQVLDLAVEMTPLSNPEALANASLNVFRWKTASYTTIAPLLLALLAAGESPDQARHCALAVGRPLGLAFQLADDLLDVVGSSRNTGKPVGGDIREGKRTVLLADALSAADTADKADLIAIFEEDCRNDNQVARTIELFTSTGALDRSRERIAALWGESRKAIAGLELNSEAQRRLTEACARFVPESLR

SEQ ID NO: 49 - protein, 291 aa, Artificial Sequence
  Description of Artificial Sequence Synthetic polypeptide
MSDKIKKMGEEIELWLKEYLDNKGNYDKKIYEAMAYSLEAGGKRIRPVLFLNTYSLYKEDYKKAMPIAAAIEMIHTYFLIHDDLPAMDNDDLRRGKPTNHKIFGEAIAILAGDALLNEAMNIMFEYSLKNGEKALKACYTIAKAAGVDGMIGGQVVDILSEDKSISLDELYYMHKKKTGALIKASILAGAILGSATYTDIELLGEYGDNLGLAFQIKDDILDVEGDTTTLGKKTKSDEDNHKTTFVKVYGIEKCNELCTEMTNKCFDILNKIKKNTDKLKEITMFLLNRNY

SEQ ID NO: 50 - protein, 301 aa, Artificial Sequence
  Description of Artificial Sequence Synthetic polypeptide
MSKKRKTLEDTAMNINSLKEEVDQSLKAYFNKDREYNKVLYDSMAYSINVGGKRIRPILMLLSYYIYKSDYKKILTPAMAIEMIHTYFIHDDLPCMDNDDLRRGKPTNHKVFGEAIAVLAGDALLNEAMKILVDYSLEEGKSALKATKIIADAAGSDGMIGGQIVDIINEDKEEISLKELDYMHLKKTGELIKASIMSGAVLAEASEGDIKKLEGFGYKLGLAFQIKDDILDVVGNAKDLGKNVHKDQESNKNNYITIFGLEECKKKCVNITEECIEILSSIKGNTEPLKVLTMKLLERKF

SEQ ID NO: 51 - protein, 300 aa, Artificial Sequence
  Description of Artificial Sequence Synthetic polypeptide
MSDFPQQLEACVKQANQALSRFIAPLPFQNTPVVETMQYGALLGGKRLRPFLVYATGHMFGVSTNTLDAPAAAVECIHAYFLIHDDLPAMDDDDLRRGLPTCHVKFGEANAILAGDALQTLAFSILSDADMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQSLKQLAEQSLDTSALEALADYIIQRNK

SEQ ID NO: 52 - protein, 294 aa, Artificial Sequence
  Description of Artificial Sequence Synthetic polypeptide
MSTNFSQQHLPLVEKVMVDFIAEYTENERLKEAMLYSIHAGGKRLRPLLVLTTVAAFQKEMETQDYQVAASLEMIHTYFLIHDDLPAMDDDDLRRGKPTNHKVFGEATAILAGDGLLTGAFQLLSLSQLGLSEKVLLMQQLAKAAGNQGMVSGQMGDIEGEKVSLTLEELAAVHEKKTGALIEFALIAGGVLANQTEEVIGLLTQFAHHYGLAFQIRDDLLDATSTEADLGKKVGRDEALNKSTYPALLGIAGAKDALTHQLAEGSAVLEKIKANVPNFSEEHLANLLTQLQLR

SEQ ID NO: 53 - protein, 304 aa, Artificial Sequence
  Description of Artificial Sequence Synthetic polypeptide
MSSSPNLSFYYNECERFESFLKNHHLHLESFHPYLEKAFFEMVLNGGKRFRPKLFLAVLCALVGQKDYSNQQTEYFKIALSIECLHTYFLIHDDLPCMDNAALRRNHPTLHAKYDETTAVLIGDALNTYSFELLSNALLESHIIVELIKILSANGGIKGMILGQALDCYFENTPLNLEQLTFLHEHKTAKLISASLIMGLVASGIKDEELFKWLQAFGLKMGLCFQVLDDIIDVTQDEEESGKTTHLDSAKNSFVNLLGLERANNYAQTLKTEVLNDLDALKPAYPLLQENLNALLNTLFKGKT

SEQ ID NO: 54 - protein, 301 aa, Artificial Sequence
  Description of Artificial Sequence Synthetic polypeptide
MSPINARLIAFEDQWVPALNAPLKQAILADSHDAQLAAAMTYSVLAGGKRLRPLLTVATMRSLGVTFVPERHWRPVMALELLHTYFLIHDDLPAMDNDALRRGEPTNHVKFGAGMATLAGDGLLTLAFQWLTATDLPATMQAALVQALATAAGPSGMVAGQAKDIQSEHVNLPLSQLRVLHKEKTGALLHYAVQAGLILGQAPEAQWPAYLQFADAFGLAFQIYDDILDVVSSPAEMGKATQKDADEAKNTYPGKLGLIGANQALIDTIHSGQAALQGLPTSTQRDDLAAFFSYFDTERVN

SEQ ID NO: 55 - protein, 286 aa, Artificial Sequence
  Description of Artificial Sequence Synthetic polypeptide
MSDTKILKLEDFLTEFYESAEFPTGLAESAKYSLLAGGKRIRPLLFLNLLEAFDLELSKAHYHVAAALEMIHTGSLIHDDLPAMDNDDYRRGQLTNHKKFDEATAILAGDTLFFDPFFILSTADLSAEIIVALTRELAFASGSYGMVAGQILDMAGEGKELTLAEIEQIHRLKTGRLLTFPFVAAGIVAQKSTDEVEKLRQVGQILGLAFQIRDDILDVTATFAELGKTPGKDILEEKSTYVAHLGLEGAKKSLTGNLSEVKKLLTDLSVTDSSEIFKIIEQLEVK

SEQ ID NO: 56 - protein, 305 aa, Artificial Sequence
  Description of Artificial Sequence Synthetic polypeptide
MSIDLKSFQKEWLPKINQQLENDLSMASPDADLVAMMKYAVLNGGKRLRPLLTLAVVTSFGESITPSILKVATAIEWVHSYFLVHDDLPAMDNDMFRRGKPSVHALYGEANAILVGDALLTGAFGVIATANSSCSVEDCLPTEELLLITQNLAREAGGSGMVLGQLHDMDNHTEEQNASTNWLLNDVYSMKTAALIRYTTTLGAILTHQNVNVEDNHFDPKKAMYDFGEKFGLAFQIQDDLDDYQQDQLEDVNSLPHIVGVKEAQSVLDQYLFSTQEILANTVEQDQQFDRRLLDDFVSLIGDKK

SEQ ID NO: 57 - protein, 295 aa, Artificial Sequence
  Description of Artificial Sequence Synthetic polypeptide
MSQDLTLFLEQYKKVIDESLFKEISERNIEPRLKESMLYSVQAGGKRIRPMLVFATLQALKVNPLLGVKTATALEMIHFTYFLIHDDLPAMDNDDYRRGKYTNHKVFGDATAILAGDALLTLAFSILAEDENLSFETRIALINQISFSSGAEGMVGGQLADMEAENKQVTLEELSSIHARKTGELLIFAVTSAAKIAEADPEQTKRLRIFAENIGIGFQISDDILDVIGDETKMGKKTGVDAFLNKSTYPGLLTLDGAKRALNEHVAIAKSALSGHDFDDEILLKLADLIALREN

SEQ ID NO: 58 - protein, 350 aa, Artificial Sequence
  Description of Artificial Sequence Synthetic polypeptide
MSTGAITEQLRRYLHDRRAETAYIGDDYSGLIAALEEFVLNGGKRLRPAFAYWGWRAVATEAPDDQALLLFSALELLHACALVHDDVIDDSATRRGRPTTHVRFASLHRDRQWQGSPERFGMSAAILLGDLALAWADDIVLGVDLTPQAARRVRRVWANIRTEVLGGQYLDIVAEASAAASIASAMNVDTFKTACYTVSRPLQLGAAAAADRPDVHDLFSQFGTDLGVAFQLRDDVLGVFGDPAVTGKPSGDDLRSGKRTVLLAEAVELAEKSDPLAAKLLRDSIGAQLSDAEVDRLRDVIESVGALAAAEQRIATLTQRALATLAAAPINTAAKAGLSELAKLATNRSA

SEQ ID NO: 59 - protein, 326 aa, Artificial Sequence
  Description of Artificial Sequence Synthetic polypeptide
MSIPAVSLGDPQFTANVHDGIARITELINSELSQADEVMRDTVAHLVDAGGTPFRPLFTVLAAQLGSDPDGWEVTVAGAAIELMHLGTLCHDRVVDESDMSRKTPSDNTRWTNNFAILAGDYRFATASQLASRLDPEAFAVVAEAFAELITGQMRATRGPASHIDTIEHYLRVVHEKTGSLIAASGQLGAALSGAAEEQIRRVARLGRMIGAAFEISRDIIAISGDSATLSGADLGQAVHTLPMLYALREQTPDTSRLRELLAGPIHDDHVAEALTLLRCSPGIGKAKNVVAAYAAQAREELPYLPDRQPRRALATLIDHAISACD

SEQ ID NO: 60 - protein, 276 aa, Artificial Sequence
  Description of Artificial Sequence Synthetic polypeptide
MSKFKDFSNRYLPEINNDLSNYFADRDDDIFRMITYALNSTGKRLRPLLTLATFAAAGNVINDSTIEAATAVEFVHAYFLVHDDLPEMDDDTKRRNQSSTWKKFGVGNAVLVGDGLLTEAFKKISNLSLPESIRLRLIYNLALAAGPDNMVRGQQYDLFSQDKVESIDDLEFIHLMKTGALMTYAATAGGILAGLSDDKLRALNIYGANLGIAFQIKDDLRDIKQDEEENKKSFPRLIGVQKSQTELEEHLKISANAIKEIPDFQNTVLLDLLDRI

SEQ ID NO: 61 - protein, 374 aa, Artificial Sequence
  Description of Artificial Sequence Synthetic polypeptide
MSEAVLSAGAGESTRPSPSVPPFTDTVEDALREFFASRAGTVETVGGGYAEAVAALESFVLRGGKRVRPMFVWTGWLGAGGDATGPEAPAALRAASALELVQACALVHDDIIDASTTRRGFPTVHVEFADQHSAHHWSGGSAEFGRAVAILLGDLALAWADDMIREAGLSPDAQARISPVWSAMRTEVLGGQFLDISSEVRGDETVEAALRVDRYKTAAYTIERPLHLGAALAGADDALVAAYRTFGTDIGIAFQLRDDLLGVFGDPEITGKPSGDDLRAGKRTVLFAEALQRADASDPAAAALLRESIGTDLSDAQVATLRSVITDLGAVDDAERRISELTDSALSALDGSTATDEGKLRLREMAIAVTRRDA

SEQ ID NO: 62 - protein, 300 aa, Artificial Sequence
  Description of Artificial Sequence Synthetic polypeptide
MSDFPQQLEACVKQANQALSRFIAPLPFQNTPVVETMQYGALLGGKRLRPFLVYATGHMFGVSTNTLDAPAAAVECIHAYFLIHDDLPAMDDDDLRRGLPTCHVKFGEANAILAGDALQTLAFSILSDANMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQALKQLAEQSLDTSALEALADYIIQRNK

SEQ ID NO: 63 - protein, 290 aa, Artificial Sequence
  Description of Artificial Sequence Synthetic polypeptide
MSKDKIKYINQAIKHYYAQTHVSQDLVEAVLYSVAAGGKRIRPLLLLEILQGFGLVLTEAHYQVAASLEMIHTGFLVHDDLPAMDNDDYRRGQLTNHKKFGETTAILAGDSLFLDPFGLLAKADLRADIKIKLVAELSDAAGSYGMVGGQMLDIKGEHVQLNLDQLAQIHANKTGKLLTFPFVAAGIIAELSEKALARLRQVGELVGLAFQVRDDILDVTASFSELGKTPQKDIEADKSTYPSLLGLDKSYAILEDSLNQAQAIFQKLALEEQFNATGIETIIERLRLHA

SEQ ID NO: 64 - protein, 295 aa, Artificial Sequence
  Description of Artificial Sequence Synthetic polypeptide
MSQEALISFQQRNNQQLEWWLSQLPHQNQTLIEAMRYGLLLGGKRARPFLVYITGQMLGCKAEDLDTPASAVECIHAYSLIHDDLPAMDDDELRRGQPTCHIKFDEATAILTGDALQTLAFSILADGPLNPNAESMRINMVKVLAQASGAAGMCMGQALDLQAENRLVNLQELEEIHRNKTGALMKCAIRLGALAAGEKGREVLPLLDKYADAIGLAFQVQDDILDIISDTETLGKPQGSDQELNKSTYPALLGLEGAIEKANNLLQEALQALDAIPYNTELLEEFARYVIERKN

SEQ ID NO: 65 - protein, 317 aa, Artificial Sequence
  Description of Artificial Sequence Synthetic polypeptide
MSHKPVDLTDTAAFETQLDRWRGRIGEAVAEAMAFGTTVPAPLQAGMSHAVLAGGKRYRGMLVLALGSDLGVPEEQLLSSAVAIETIHAASLVVDDLPCMDDARRRRSQPATHVAFGEATAILSSIALIARAMEVVARDRQLSPASRSSIVDTLSHAIGPQALCGGQYDDLYPPYYATEQDLIHRYQRKTSALFVAAFRCPALLAEVDPETLLRIARAGQRLGVAFQIFDDLLDLTGDAHAIGKDVGQDHGTVTLATLLGPARAAERAADELAAVQKELRETVGPGRALDLIRRMAARIAGTGKKSAGRDDLRPHAG

SEQ ID NO: 66 - protein, 289 aa, Artificial Sequence
  Description of Artificial Sequence Synthetic polypeptide
MSAFEQRIEAAMAAAIARGQGSEAPSKLATALDYAVTPGGARIRPTLLLSVATRCGDSRPALSDAAAVALELIHCASLVHDDLPCFDDAEIRRGKPTVHRAYSEPLAILTGDSLIVMGFEVLAGAAADRPQRALQLVTALAVRTGMPMGICAGQGWESESQINLSAYHRAKTGALFIAATQMGAIAAGYEAEPWEELGARIGEAFQVADDLRDALCDAETLGKPAGQDEIHARPSAVREYGVEGAAKGLKDILGGAIASIPSCPAEAMLAEMVRRYADKIVPAQVAARV

SEQ ID NO: 67 - protein, 362 aa, Artificial Sequence
  Description of Artificial Sequence Synthetic polypeptide
MSALTLPDAQPPTGLLPLEQAWLQLVQTEVETSLAELFELPDEAGLDVRWTQALTQARAYTLRPAKRLRPALVMAGHCLARGSAVVPSGLWRFAAGLELLHTFLLIHDDVADQAELRRGAPPLHRMLAPGRAGEDLAVVVGDHLFARALEVMLGSGLTCVAGVVQYYLGVSGHTAAGQYLDLDLGRAPLAEVTLFQTLRVAHLKTARYGFCAPLVCAAMLGGASSGLVEELERVGRHVGLAYQLRDDLLGLFGDSNVAGKAADGDFLQGKRTFPVLAAFARATEAERTELEALWALPVEQKDAAALARARALVESCGGRAACERMVVRASRAARRSLQSLPNPNGVRELLDALIARLAHRAA

SEQ ID NO: 68 - protein, 380 aa, Artificial Sequence
  Description of Artificial Sequence Synthetic polypeptide
MSEATLSAGTARVGQSSTNTAPHPTSLELPGVFEGALRDFFDSRRELVSNIGGGYEKAVSTLEAFVLRGGKRVRPSFAWTGWLGAGGDPNGSGADAVIRACAALELVQACALVHDDIIDASTTRRGFPTVHVEFEDQHRGEEWSGDSAHFGEAVAILLGDLALAWADDMIRESGISPDAAARVSPVWSAMRTEVLGGQFLDISNEARGDETVEAAMRVNRYKTAAYTIERPLHLGAALFGADAELIDAYRTFGTDIGIAFQLRDDLLGVFGDPSVTGKPSGDDLIAGKRTVLFAMALARADAADPAAAELLRNGIGTQLTDNEVDTLRQVITDLGAVTDVETQIDTLVEAAANALDSSTATAESKARLTDMAIAATKRSY

SEQ ID NO: 69 - protein, 325 aa, Artificial Sequence
  Description of Artificial Sequence Synthetic polypeptide
MSPAGALAPLADFFAAGGKRLRPTLCVLGWHAAGGQTPASREVVQVAAALEMFHAFALIHDDVMDDSDIRRGAPTLHRALAGQYADHRPRALTDRLGAGAAILIGDLALCWSDELIHTAGLRHDQFARILPVLDMMRTEVMYGQYLDVTATGQPTADIGRAQTIIRYKTAKYTIERPLQLGAELAGASTDVIDALSAYAVPLGEAFQLRDDLLGAFGDPVVTGKSSTEDLREGKPTVLVGLALRDAAPDQADVLRRLLGRRDLTEDQATQIRAVLTGTGARAQVENMIAQRRERVLALLDTNTVLDATAVFHLRQLADSATRRTS

SEQ ID NO: 70 - protein, 303 aa, Artificial Sequence
  Description of Artificial Sequence Synthetic polypeptide
MSTVCAKKHVHLTRDAAEQLLADIDRRLDQLLPVEGERDVVGAAMREGALAPGKRIRPMLLLLTARDLGCAVSHDGLLDLACAVEMVHAASLILDDMPCMDDAKLRRGRPTIHSHYGEHVAILAAVALLSKAFGVIADADGLTPLAKNRAVSELSNAIGMQGLVQGQFKDLSEGDKPRSAEAILMTNHFKTSTLFCASMQMASIVANASSEARDCLHRFSLDLGQAFQLLDDLTDGMTDTGKDSNQDAGKSTLVNLLGPRAVEERLRQHLQLASEHLSAACQHGHATQHFIQAWFDKKLAAVS

SEQ ID NO: 71 - protein, 327 aa, Artificial Sequence
  Description of Artificial Sequence Synthetic polypeptide
MSELDKYFDEIIKNVNEEIEKYIKGEPKELYDASIYLLKAGGKRLRPLITVASSDLFSGDRKRAYKAAAAVEILHNFTLIHDDIMDEDTLRRGMPTVHVKWGVPMAILAGDLLHAKAFEVLSEALEGLDSRRFYMGLSEFSKSVIIIAEGQAMDMEFENRQDVTEEEYLEMIKKKTAQLFSCSAFLGGLVSNAEDKDLELLKEFGLNLGIAFQIIDDILGLTADEKELGKPVYSDIREGKKTILVIKALSLASEAERKIIIEGLGSKDQGKITKAAEVVKSLSLNYAYEVAEKYYQKSMKALSAIGGNDIAGKALKYLAEFTIKRRK

SEQ ID NO: 72 - protein, 352 aa, Artificial Sequence
  Description of Artificial Sequence Synthetic polypeptide
MSTHVPANAVPTTNGLSIIPPGLSLPTTFAPLVERIQTVAHLVETAIAEDLSEVTQPELRQAVLHLFDGKGKRLRPFLVITTAEAAGGTLEAALPPALAVEYLHNLSLIHDDMMDGSPERHGRPTLHTRFGLNLSLLVGDLLYAKAVEQASRIRHHALRMVHILGQTAKQMCYGQFDDLYFERRLDLTIEDYLRMAARKTSALYRASCIFGMLTADADEADLQAMATFGENIGTAFQIWDDVLDLQADPLRLGKPLGLDIREGKKTLIVIHFLQHASPAARRRFLELLGKRDLNGELPEAIALLEETGSIAFARDLAIRYLVDAKQHLSVLPAGPHRKLLDMYADFMLQRRH

SEQ ID NO: 73 - protein, 349 aa, Artificial Sequence
  Description of Artificial Sequence Synthetic polypeptide
MSTSETKEARVLDAIRERRDLVNAAIDEELPVQEPERLYEATRYILEAGGKRLRPTVTTLAAEAVTGTEPMGADFRAFPSLDGDDVDVMRAAVAIEVIQSFTLIHDDIMDEDDLRRGVPAVHEAYDVSTAILAGDTLYSKAFEFMTETGADPQNGLEAMRMLASTCTEICEGQALDVSFESRDDILPEEYLEMVELKTAVLYGASAATPALLLGADEEVVDALYRYGIDSGRAFQIQDDVLDLTVPSEELGKQRGSDLVEGKETLITLHARQQGIDVDGLVEADTPAEVTEAAIEEAVATLAEAGSIEYARETAEDLTARSKGHLEVLPESGSRSLLEDLADYLIVRGY

SEQ ID NO: 74 - protein, 328 aa, Artificial Sequence
  Description of Artificial Sequence Synthetic polypeptide
MSETLTRYLSEFRPLVDKKIMEVLEGSPKELYEAARHLPSKGGKRLRPALVLLVNKALGGEVEGALPAAAAVELLHNFTLVHDDIMDRDELRRGVPTVHVLYGESMAILAGDLLYAKAYEALLQSPQPPDLVKEMTEVLTWSAVTVAEGQAMDMEFEKRWDVTEEEYLEMIEKKTGALFGASAALGALTANKREVKDLMKEFGLILGKAFQIKDDVLSLLGDEKVTGKPKYNDLREGKKTILVIYALRNLPRDEAERVKSVLGRETSYEALEEVAELIKRSGALDYAMKLAEEFEKRAYEILETVRFEDEEAMRALKELVDFAVKREY

SEQ ID NO: 75 - protein, 357 aa, Artificial Sequence
  Description of Artificial Sequence Synthetic polypeptide
MSGKQFNLLREKYLPQIEREIKKFFEEKISTQKDEVIVRYYEELSSYVLRGGKRFRPLALISSYYGSGSKHEGNIIRASISVELLHNSSLIHDDIMDESPKRRGGPSFHYLMANWSRLSPRTPPPRNPGISLGILGGDSLIELGLEALLESGFPNEIIVKAASEYSVAYRKLIEGQLLDLYLSTVTMPTEEEVLRMLSLKTGTLFSASLVMGGMLAGASEDMLHFLRSFGQRVGVAFQLQDDILGLYGDEAVIGKPADSDIKEGKRTLLVVKAWELSDEATRKKLLSILGNPNISAADLNYVREVVKELGALDYTRKTALNLLKENEKDIEFNKHLFEESFVEFLKELNEIVIARSF

SEQ ID NO: 76 - protein, 346 aa, Artificial Sequence
  Description of Artificial Sequence Synthetic polypeptide
MSSNINEDVGKVLGQYSKDIHKEIGNTLSNIGPEDLREASIYLTEAGGKMLRPALTVLICEAVGGTFSSCIKAAAAIELIHTFSLIHDDIMDKDDMRRGKPSVHKVWGEPVAILAGDTLFSKAYELVINSKNEIDSSNPEECLNRVNRTLSTVADACVKICEGQAQDMGFEGNFDVSEEEYMEMIFKKTAALIAAATESGAIMGGANEKIVSDMYDYGKLIGLAFQIQDDYLDLVSDEDSLGKPVGSDIAEGKMTIIVVNALNRANPEDKKRILEILRMGNESGNCDQVYVDEAISLFEKYGSIQYAQNIALANVKKAKQLLEILPESEAKHTLSLVADFVLYRQN

SEQ ID NO: 77 - protein, 323 aa, Artificial Sequence
  Description of Artificial Sequence Synthetic polypeptide
MSSDLKTYLEKTAEQVDIALERNFGDVFGDLYKASAHLLLAGGKRLRPAVLLLAANAVKPGRADDLITAAIAVEMTHTFYLIHDDIMDGDVTRRGVPTVHTKWDEPTAILAGDVLYAKSFEYITHALAEDRARVKAVTLLARTCTEICEGQHQDMAFEQKGAEVEEADYIEMAGKKTGALYAAAAAIGGTLAGGNAMQVDALYQYGMNAGIAFQIQDDLIDLLAPPETSGKDRASDLREGKQTLIAIIAREKDLDLSKYRHTLTTTEIDAAIAELEGAGVVDEVRRAAEERVATAKRALSVLPESMERTYLEEIADYFLTRSF

SEQ ID NO: 78 - protein, 322 aa, Artificial Sequence
  Description of Artificial Sequence Synthetic polypeptide
MSDLIDELKKRSTLVDESIQEFLPIDHPEELYRATRYLPDAGGKRLRPAVLMLSAEAVGGDSDSVLPAAVALELIHNFTLIHDDIMDRDDIRRGMPALHVKWGTAGAILAGDTLYSRAFEIISKMDADPQKLLKCVALLSRTCTKICEGQWLDVDFEKRDIVDVDEYLEMIENKTSVLYGAAAKVGAILGGASDEVADAMYEFGRLTGISFQIHDDVIDLVTPEEILGKSRGSDLKEGKKTLIALHALNNGVELECFGKADATQDEINNAVAKLEESGTLAYVREMADNYLEDGKSKLDLLEDSPAKETLIEIADYMVSREY

SEQ ID NO: 79 - protein, 323 aa, Artificial Sequence
  Description of Artificial Sequence Synthetic polypeptide
MSDLIEEIKKRSSHVDKGIEEYLPIDKPYELYKAARYLPDAGGKRLRPATVILAAEAVGSDLETVLPAAVAVELVHNFYLVHDDIMDRDDIRRGMPAVHVKWGEAGAILAGDTLYSKAFEILTHAPAEAPERNLKCIDILSKACRDICEGQWMDVEFENRDDVTKEEYLEMIEKKTGVLYAASMQIGAILGGAPEEVSDAFYECGRLIGIAFQIYDDVIDMTTPEEVLGKVRGSDLMEGKKTLIAIHALNKGVELKIFGKGEATTEEINEAVHQLEEAGSIDYVRDLALDYIARGKELLNVVEDSESKTILKAIADYMITRSY

SEQ ID NO: 80 - protein, 321 aa, Artificial Sequence
  Description of Artificial Sequence Synthetic polypeptide
MSIEEILQKKAKLVDESIPKFLPITPPDELYKAMRHLLDAGGKRLRPSALLLASEAVGGKPDDVLPAAVAVELVHNFTLIHDDIMDEADLRRGLATVHKKWGVPRAIIAGDALYSKAFEILSCTKSEPQRLVESLELLSKTCTDICEGQWMDMNFQTRKDVTEEEYMRMVEKKTAVLFATALKLGAVLSGANREHVRALWDFGRLTGVGFQIYDDVIDLITPEEILGKAQGGDIIEGKRTLIIIHALSKGISIDALGKCNATRSEISAALTTLKESGSIDYAMNKALSFVDEGKAALAMLPESEAKNILTRLADYMIERKY

SEQ ID NO: 81 - protein, 326 aa, Artificial Sequence
  Description of Artificial Sequence Synthetic polypeptide
MSEADMSDLSAYLKSVAQQIDGMIEKNFTHAGGELDRASAHLLSAGGKRLRPAVVMLSADAIRHGSSKDVMPAALALEVTHTFYLIHDDIMDGDSLRRGVPTVHTKWDMPTGILAGDVLYARAFEFICQSKADEGPKVQAVALLARACADICEGQHQDMSFEHRADVTEEEYMAMVAKKTGVLYAAAAAIGGTLAGGNPEQIRALYQFGLNTGIAFQIQDDLIDLLTPTEKSGKDQGSDLREGKQTLVMIIARQKGVDLLKYRHELSPADIKAAIQELTDAGVIDAVKKKAADLVADSNRLLMVLPPTKERQLIMDVGEFFVTRSF

SEQ ID NO: 82 - protein, 321 aa, Artificial Sequence
  Description of Artificial Sequence Synthetic polypeptide
MSELIEYLEKVGNQVDRLIDRYFGDPVGELNKASAHLLTAGGKRLRPAVMMLAADAVRKGSSDDLMPAAIALELTHSFYLIHDDIMDGDEVRRGVPTVNKKWDEPTAILAGDVLYARAFAFICQALAMDAAKLRAVSMLAVTCEEICAGQHLDMAFEDRDDVSEEEYLEMVGKKTGALYAASTAMGGVLAGGSQPQVDALYRYGMNIGVAFQIQDDLIDLLASPERSGKDRASDIREGKQTLINIKAREHGFDLAPYRRRLDDAEIDDLIQQLTDNGVIGEVKATAEGLVTSAGKILAILKPSDEKDLLISIGTFFVERGY

SEQ ID NO: 83 - protein, 334 aa, Artificial Sequence
  Description of Artificial Sequence Synthetic polypeptide
MSKDTTKIEVENYINKVNNHLISFLSGKPLQLYQASTHYLKSGGKRLRPIMVIKSCEMFGGTQQDALPAAAAVEFIHNFSLVHDDIMDNDDLRHGIPTVHKSFGLPLAILSGDILFSKAFQILSITNVNSIKDSSLLSMIRRLSLACVDICEGQAKDIQFSECETFPSEEEYLEMISKKTAALFNVSCSLGALSSRNATEKDVNNMSDFGKNSGIAFQLIDDLIGIAGHSKETGKAVGNDIREGKKTYPILLSIKKASELERAHILKVFGKGQCDNMSLKKAIDVISSLQIEKIVRKSAMAYIEKAMEALVNYEDSEPKKILQELSSYIVERSK

SEQ ID NO: 84 - protein, 332 aa, Artificial Sequence
  Description of Artificial Sequence Synthetic polypeptide
MSLQDYFNEVINQVNKTIEKYLSNAPSGTSSLYEASKHLFSAGGKRLRPLILVSSCDFLGGDRSRAILAGSAIETLHTFTLIHDDIMDHDFLRRGLPTVHVKWGESMAILAGDLLHAKAFEMLNDSLEGVNETLHYEVMKTFINSIVVVSEGQAMDMQFEGRNDVTEEDYLEMVKKKTAYLIATSSKIGSLIGGAGPDVADKFFHFGIYLGIAFQIVDDIIGITSDEAELGKPLFSDIREGKRTLLVIRTLKEAESRELEVLKQVLGNKNASTDQLKEASQIVKKHSLEYAYSLAEEYRSRAISSLDGIQPRNQEAYEALKFVSEFTLKRKK

SEQ ID NO: 85 - protein, 328 aa, Artificial Sequence
  Description of Artificial Sequence Synthetic polypeptide
MSSFNSISKTAKKVNSFLLSSLHGNPEEIYKAASYLIEYGGKRLRPYMVIKSCEILGGTIKQALPSAAAIEMVHNFTLIHDDIMDNDEIRHGVSTTHKKFGIPVGILAGDVLFSKAFETISHGDPKMPKDVRLALVSNLAKACTDVCEGQALDIMMAKSQKIPTEEQYIMMIEKKTSALFAAACAMGAISANTKTRDVTNLSSFGKNLGVAFQIVDDLIGIIGDSKITKKPVGNDLREGKKSLPILLAINKVSGKKKEIILNAFGNSAISKKELENAVRIISSMGIETAVRKKAIQYSNAAKKSLSNYKGSAKNELLSLLDFVVERSQ

SEQ ID NO: 86 - protein, 344 aa, Artificial Sequence
  Description of Artificial Sequence Synthetic polypeptide
MSGKYDELFAQVKAKAKDVDAVIFELIPEKEPKTLYEAARHYPLAGGKRVRPFVVLRAAEAVGGDPEKALYPAAAVEFIHNYSLVHDDIMDMDELRRGRPTVHKLWGVNMAILAGDLLFSKAFEAVARAEVSPEKKARILDVLVKTSNELCEGQALDIEFETRDEVTVDEYLKMISGKTGALFNGSATIGAIVGTDNEKYIQALSKWGRNVGIAFQIWDDVLDLIADEEKLGKPVGSDIRKGKKTLIVSHFFQHANEEDKAEFLKVFGKYAGDAKGDALIHDEKVKEEVAKAIELLKKYGSIDYAANYAKNLVREANEALKVLPESEARKDLELLAEFLVEREF

SEQ ID NO: 87 - protein, 328 aa, Artificial Sequence
  Description of Artificial Sequence Synthetic polypeptide
MSDIISRFSEKIDAVNSAIDKFLRIREPKRLYSATRHLPLAGGKRLRPILAMLSTEAVGEDWKKTIPFAVSLELLHNFTLVHDDIMDRSDLRRGIETVHVKFGEPTAILAGDILFAKSFEVLYELDIDDAIFKTVNRLLIDCIEEICDGQQIDMEFESRKYVSEEEYLEMIEKKTSALFSCATTGGAIIGDGNNREVDSLSLYGRFFGLAFQIWDDYLDIAGEEGEFGKKIGNDIRCGKKTLMIVHATKNADGREKETIFSILGKKDATDEEINEVMEILTKSGSIDYAKKKALHFAEKAKEQLRVLPDSRAKRDLIELVDFAISRER

SEQ ID NO: 88 - protein, 319 aa, Artificial Sequence
  Description of Artificial Sequence Synthetic polypeptide
MSLIDHYIMDFMSITPDRLSGASLHLIKAGGKRLRPLITLLTARMLGGLEAEARAIPLAASIETAHTFSLIHDDIMDRDEVRRGVPTTHVVYGDDWAILAGDTLHAAAFKMIADSREWGMSHEQAYRAFKVLSEAAIQISRGQAYDMLFEETWDVDVADYLNMVRLKTGALIEAAARIGAVAAGAGSEIEKMMGEVGMNAGIAFQIRDDILGVIGDPKVTGKPVYNDLRRGKKTLLVIYAVKKAGRREIVDLIGPKASEDDLKRAASIIVDSGALDYAESRARFYVERARDILSRVPAVDAESKELLNLLLDYIVERVK

SEQ ID NO: 89 - protein, 320 aa, Artificial Sequence
  Description of Artificial Sequence Synthetic polypeptide
MSISEIIKDRAKLVNEKIEELLKEQEPEGLYRAARHYLKAGGKRLRPVITLLSAEALGEDYRKAIHAAIAIETVHNFTLVHDDIMDEDEMRRGVKTVHTLFGIPTAILAGDTLYAEAFEILSMSDAPPENIVRAVSKLARVCVEICEGQFMDMSFEERDSVGESEYLEMVRKKTGVLIGISASIPAVLFGKDESVEKALWNYGIYSGIGFQIHDDLLDISGKGKIGKDWGSDILEGKKTLIVIKAFEEGIELETFGKGRASEEELERDIKKLFDCGAVDYARERAREYIEMAKKNLEVIDESPSRNYLVELADYLIERDH

SEQ ID NO: 90 - protein, 347 aa, Artificial Sequence
  Description of Artificial Sequence Synthetic polypeptide
MSSERHQQVEDAIVARRDRVNDALPEDLPVKKPDHLYEASRYLLDAGGKRLRPTVLLLVAESLLDVDPLTADYRDFPTLGGGQADMMSAALAIEVIQTFTLIHDDIMDDDALRRGVPAVHKEYDLSTAILAGDTLYSKAFEFLLGTGAAHERTVEANKRLATTCTRICEGQSLDIEFEQRDVVTPEEYLEMVELKTAVLYGAAASIPATLLGADAETVDALYNYGLDVGRAFQIQDDLLDLTTPSEKLGKQRGSDLVENKQTLVTLHARQQGVDVGDLIDTDSVEAVSEAEIDAAVERLREVGSIEYARQTGQDLIASGKQNLEVLPDNESRSLLEGIANYLVERDY

SEQ ID NO: 91 - protein, 325 aa, Artificial Sequence
  Description of Artificial Sequence Synthetic polypeptide
MSMLMTLVDEIKNRSSHVDAAIDELLPVTRPEELYKASRYLVDAGGKRLRPAVLILAAEAVGSNLRSVLPAAVAVELVHNFTLIHDDIMDRDDIRRGMPAVHVKWGEAGAILAGDTLYSKAFEILSKVENEPVRVLKCMDVLSKTCTEICEGQWLDMDFETRKKVTESEYLEMVEKKTSVLYAAAAKIGALLGGASDEVAEALSEYGRLIGIGFQMYDDVLDMTAPEEVLGKVRGSDLMEGKYTLIVINAFEKGVKLDIFGKGEATLEETEAAVRTLTECGSLDYVKNLAISYIEEGKEKLDVLRDCPEKTLLLQIADYMISREY

SEQ ID NO: 92 - protein, 326 aa, Artificial Sequence
  Description of Artificial Sequence Synthetic polypeptide
MSTEVLDILRKYSEVADKRIMECISDITPDTLLKASEHLITAGGKKIRPSLALLSCEAVGGNPEDAAGVAAAIELIHTFSLIHDDIMDDDEMRRGEPSVHVIWGEPMAILAGDVLFSKAFEAVIRNGDSERVKDALAVVVDSCVKICEGQALDMGFEERLDVTEDEYMEMIYKKTAALIAAATKAGAIMGGASEREVEALEDYGKFIGLAFQIHDDYLDVVSDEESLGKPVGSDIAEGKMTLMVVKALEEASEEDRERLISILGSGDEGSVAEAIEIFERYGATQYAHEVALDYVRMAKERLEILEDSDARDALMRIADFVLEREH

SEQ ID NO: 93 - DNA, 1164 bp, Artificial Sequence
  Description of Artificial Sequence Synthetic polynucleotide
atgaagatcgaagaaggaaagttagtgatctggataaatggtgataaaggctacaatgggttggcggaagtaggaaaaaagttcgagaaagacacaggaatcaaagttacggtcgagcaccccgataaactagaggaaaagtttccacaggtagctgctacgggggacggaccagacattatcttttgggcccacgatagattcgggggttatgctcagtccggacttctggccgagattactccagacaaggccttccaagacaaactttacccgttcacatgggacgcagtcaggtacaatggaaagctgattgcatatccgatagctgtggaggcacttagcctaatttacaacaaggatctactacctaaccctcccaagacttgggaagaaattccagctctggacaaggagttaaaagcaaagggtaagagtgcacttatgttcaatctacaagagccttatttcacatggcccctaatagccgccgacggaggctatgcctttaagtacgaaaacggcaagtatgacataaaggatgttggggtagacaacgcgggagccaaggctggattaactttcctggtggatttaattaagaacaaacacatgaacgcagacactgactactctatcgcagaagcagcgttcaataaaggcgaaacggcgatgacaattaacgggccctgggcttggtcaaacattgacacgagtaaagttaactatggtgtaacggtattgcccacatttaagggacaacccagtaaacctttcgtaggagtcttgtcagccgggatcaatgcagcttccccgaataaagagcttgctaaggaatttcttgaaaattatcttttaaccgatgagggattggaggcggttaacaaggacaagcctcttggtgctgtagccctgaaatcctatgaagaagagttagctaaggacccaagaatcgccgcaacaatggagaatgctcagaagggagaaattatgccaaatataccacaaatgagtgccttctggtatgcggtaaggacggcagttattaatgccgcttcaggtagacaaacagtcgatgaggctttgaaagatgcacagactaacagttcatccaacaataataacaataacaataacaataacctgggtatcgagggccgttaa

SEQ ID NO: 94 - DNA, 717 bp, Artificial Sequence
  Description of Artificial Sequence Synthetic polynucleotide
atggtatctaaaggagaagaattgtttacaggcgtggtaccaattctggttgaattggacggtgacgtgaacggacacaaattcagcgtgagtggagaaggcgagggagatgctacctatggcaagttgacgcttaaactgatctgcacaacgggcaaattaccagtgccctggccgacgcttgtaacaactcttggatacgggttacagtgctttgcccgttatccagaccatatgaaacagcatgacttcttcaaatctgcgatgccggagggatatgtacaggaacgtacgattttctttaaggacgatgggaactacaagactcgtgctgaggttaagtttgaaggcgacactctagtcaataggatagaattaaagggtattgattttaaggaggatgggaacatcctgggccataaactagagtacaactacaattcacataatgtctacatcaccgctgataaacagaagaacgggatcaaagctaatttcaagatacgtcataatatcgaagatggtggcgtccagcttgctgaccactaccagcagaacacgcctataggcgacgggccggtgttgctacctgacaatcattatctgtcctatcagtccgccctttcaaaagaccctaatgagaagagggatcatatggtgcttttagaatttgtaaccgcggcagggatcacacttgggatggatgagctgtataaa

SEQ ID NO: 95 - DNA, 351 bp, Artificial Sequence
  Description of Artificial Sequence Synthetic polynucleotide
atggcgatgttctgtaccttctttgagaaacatcatagaaaatgggacatcttactagaaaagagcaccggagtgatggaggcgatgaaagtaacttcagaagagaaagagcagttgtctacagctatcgatagaatgaatgaaggtctggacgcatttattcaactatataacgaatccgagatcgatgaacctttaatccagttggatgacgatacagcagaactaatgaaacaggctagggacatgtacggccaagagaaacttaacgagaaattaaacacaataatcaaacaaatcctgtcaatttctgtctccgaagagggtgagaaagaaggaagcggatcaggc

SEQ ID NO: 96 - DNA, 282 bp, Artificial Sequence
  Description of Artificial Sequence Synthetic polynucleotide
atgtacctacttgggattggacttattctggcgcttattgcttgtaagcaaaatgtttccagcctagatgaaaaaaattccgtgtctgtcgatcttcctggcgaaatgaaggttttagtatccaaggagaaaaataaggacggcaaatacgacttgattgcgacagtcgataaactagagctaaaaggcacgagcgataaaaataacggctctggagtgttagaaggggtaaaagcagataaaagcaaggtcaagctgaccatatcagatgatggatcaggc

SEQ ID NO: 97 - DNA, 525 bp, Artificial Sequence
  Description of Artificial Sequence Synthetic polynucleotide
atggcggacagggacaggtcaggtatctatgggggggctcatgcgacctatgggcaacagcagcagcagggaggtggtggacgtccgatgggagaacaagttaagggcatgttacacgacaaaggtcccactgcctcccaagcattgaccgttgcaacattgttcccattgggcggacttttattagtcctttctggcctggctctaactgcaagcgtggtaggcctagctgtagccacacccgtgttcttgattttttctccggtccttgtaccggcggctttactgatcggtactgctgtaatgggtttcctaacatccggggccttagggttaggggggttgtcatccttaacctgcctagcgaacaccgccaggcaggcgtttcagcgtactcccgattacgtcgaggaagcccacaggagaatggctgaggctgcggcgcatgcgggacataaaactgcccaggcaggacaagctattcagggccgtgcacaggaggcaggagccggcggaggcgcggga

SEQ ID NO: 98 - protein, 387 aa, Artificial Sequence
  Description of Artificial Sequence Synthetic polypeptide
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGR

SEQ ID NO: 99 - protein, 239 aa, Artificial Sequence
  Description of Artificial Sequence Synthetic polypeptide
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYK

SEQ ID NO: 100 - protein, 117 aa, Artificial Sequence
  Description of Artificial Sequence Synthetic polypeptide
MAMFCTFFEKHHRKWDILLEKSTGVMEAMKVTSEEKEQLSTAIDRMNEGLDAFIQLYNESEIDEPLIQLDDDTAELMKQARDMYGQEKLNEKLNTIIKQILSISVSEEGEKEGSGSG

SEQ ID NO: 101 - protein, 94 aa, Artificial Sequence
  Description of Artificial Sequence Synthetic polypeptide
MYLLGIGLILALIACKQNVSSLDEKNSVSVDLPGEMKVLVSKEKNKDGKYDLIATVDKLELKGTSDKNNGSGVLEGVKADKSKVKLTISDDGSG

SEQ ID NO: 102 - protein, 175 aa, Artificial Sequence
  Description of Artificial Sequence Synthetic polypeptide
MADRDRSGIYGGAHATYGQQQQQGGGGRPMGEQVKGMLHDKGPTASQALTVATLFPLGGLLLVLSGLALTASVVGLAVATPVFLIFSPVLVPAALLIGTAVMGFLTSGALGLGGLSSLTCLANTARQAFQRTPDYVEEAHRRMAEAAAHAGHKTAQAGQAIQGRAQEAGAGGGAG

(19) United States
(12) Patent Application Publication
(10) Pub. No.: US 2024/0124905 A1
(43) Pub. Date: Apr. 18, 2024

(54) Recombinant Polyprenol Diphosphate Synthases

(71) Applicant: CB THERAPEUTICS, INC., Carlsbad, CA (US)

(72) Inventors: Erin Marie SCOTT, Encinitas, CA (US); Kirsten TANG, Carlsbad, CA (US); Jacob Michael VOGAN, San Diego, CA (US)

(21) Appl. No.: 18/274,445

(22) PCT Filed: Jan. 26, 2022
(86) PCT No.: PCT/US2022/013857

§ 371 Date: Jul. 26, 2023

Related U.S. Application Data
(60) Provisional application No. 63/141,486, filed on Jan. 26, 2021.

(51) Int. Cl. C12P 7/42; C12N 9/10; C12N 15/81; C12P 17/06

(52) U.S. Cl. CPC C12P 7/42; C12N 9/1085; C12N 15/81; C12P 17/06; C12Y 205/01029; C12N 2800/102

(57) ABSTRACT

Provided is a nucleic acid comprising a recombinant bacterial or archaeal geranyl pyrophosphate synthase (GPPS) gene, codon optimized for production in yeast. Also provided is a yeast cell comprising an expression cassette comprising the above nucleic acid. Additionally provided is a method of producing a terpene or cannabinoid in a yeast, the method comprising incubating the above yeast cell in a manner sufficient to produce the terpene or cannabinoid.

### CROSS-REFERENCE TO RELATED APPLICATION

This application claims the benefit of U.S. Provisional Application No. 63/141,486, filed Jan. 26, 2021, and incorporated by reference herein in its entirety.

### SEQUENCE LISTING

The instant application contains a Sequence Listing which has been submitted electronically in ASCII format and is hereby incorporated by reference in its entirety. Said ASCII copy, created on Jan. 25, 2022, is named CBTH-11-PCT_SL.txt and is 215,720 bytes in size.

### BACKGROUND OF THE INVENTION

### (1) Field of the Invention

The present application generally relates to recombinant enzymes and genes encoding those enzymes. More specifically, the application provides recombinant geranyl pyrophosphate synthase genes and enzymes that function in yeast.

### (2) Description of the Related Art

Cannabinoids are a class of organic small molecules of meroterpenoid structures found in the plant genus Cannabis. The small molecules are currently under investigation as therapeutic agents for a wide variety of health issues, including epilepsy, pain, and other neurological problems, and mental health conditions such as depression, PTSD, opioid addiction, and alcoholism.

While it is known that cannabinoids may be obtained via biosynthesis in plant species, there are many problems associated with the synthesis of such molecules which need to be overcome, including problems with large-scale manufacturing, purification, and heterologous expression for biosynthesis.

Terpenes and related terpenoids are another class of organic small molecules of commercial value. Terpenes may be used for flavors, fragrances, and are the major component of essential oils. Like cannabinoids, they are mostly produced in plants and are subject to the same difficulties as cannabinoids when produced in large quantities. Similarly, other plant derived terpenes may be produced from the same precursor molecules. These include alkaloids like salvinorin, carotenoids and mono, sequi and diterpenoids.

Producing terpenoids, including cannabinoids, in recombinant yeast is a promising solution to the above problems. See, e.g., U.S. patent application Ser. Nos. 16/553,103, 16/553,120, 16/558,973, 17/068,636 and 63/053,539; U.S. Pat. No. 10,435,727; and US Patent Publications 2020/0063170 and 2020/0063171, all incorporated by reference.

### BRIEF SUMMARY OF THE INVENTION

Provided is a nucleic acid comprising a recombinant bacterial or archaeal geranyl pyrophosphate synthase (GPPS) gene, codon optimized for production in yeast.

Also provided is a yeast cell comprising an expression cassette comprising the above nucleic acid. In these embodiments, the yeast cell is capable of expressing a recombinant GPP synthase encoded by the above nucleic acid.

Additionally provided is a method of producing a terpene or a cannabinoid in a yeast, the method comprising incubating the above yeast cell in a manner sufficient to produce the terpene or cannabinoid.

### BRIEF DESCRIPTION OF THE SEVERAL VIEWS OF THE DRAWINGS

FIG. 1 depicts the mevalonate biosynthesis pathway that generates precursors for recombinant GPPS to produce GPP, NPP, FPP, and GGPP

FIGS. 2A, 2B, 2C and 2D depict the following terpenoid compounds which result from expression of recombinant GPPSes FIG. 2A: pyrophosphate terpenoids; FIG. 2B: monoterpenes;

FIG. 2C: sesquiterpenes; and FIG. 2D: diterpenes.

FIGS. 3A, 3B, 3C and 3D depict the cannabinoid biosynthesis pathway resulting from expression of recombinant GPPS. FIG. 4A: The alkyresorcinolic acid prenyl acceptor; FIG. 2B: the key polyprenol diphosphate prenyl donors from recombinant GPPSes; FIG. 2C: cannabinoid compounds; FIG. 2D: secondary cannabinoid products.

FIGS. 4A, 4B and 4C depict a clustal maps comparing similarity among the recombinant bkGPPSes (FIG. 4A); rkGPPSes (FIG. 4B); and both the bkGPPSes and the rkGPPSes (FIG. 4C).

FIG. 5 depicts modified host cells expressing recombinant GPPS with single and mixed bacterial and/or archaeal GPPSes combined with terpene and cannabinoid biosynthesis pathways to generate terpenes and cannabinoid products.

FIGS. 6A, 6B and 6C depict bar graphs of a modified host strain expressing recombinant GPPSes to produce cannabinoids (FIG. 6A); sesquicannabinoids (FIG. 6B); and terpenes (FIG. 6C).

FIGS. 7A and 7B depict HPLC chromatograms and UV-vis spectra of isolated CBGA (FIG. 7A); and CBGVA (FIG. 7B) produced by a modified host strain expressing recombinant GPPS.

FIG. 8 depicts HPLC chromatograms and UV-vis spectra of selective and finetuned production of cannabinoid and sesquicannabinoid products by recombinant GPPS

FIGS. 9A and 9B depict HPLC chromatograms of UV-vis spectra of terpene production via recombinant GPPS such as the monoterpene geraniol (FIG. 9A); and the diterpene geranylgeraniol (FIG. 9B).

FIG. 10 depicts the supply of GGPP from recombinant GPPSes as precursor for kolavenol and salvinorin A.

FIG. 11 depicts the supply of GPP from recombinant GPPSes as precursor for monoterpenes such as thujone.

FIGS. 12A and 12B depict GGPP products from recombinant GPPSes that can supply beta-carotene and retinoic acid pathways.

FIG. 13 depicts the supply of GGPP from recombinant GPPSes as an intermediate for diterpenes such as astaxanthin.

### DETAILED DESCRIPTION OF THE INVENTION

### Abbreviations and Definitions

To facilitate understanding of the invention, a number of terms and abbreviations as used herein are defined below as follows:

Conservative amino acid substitutions: As used herein, when referring to mutations in a protein, “conservative amino acid substitutions” are those in which at least one amino acid of the polypeptide encoded by the nucleic acid sequence is substituted with another amino acid having similar characteristics. Examples of conservative amino acid substitutions are ser for ala, thr, or cys; lys for arg; gln for asn, his, or lys; his for asn; glu for asp or lys; asn for his or gln; asp for glu; pro for gly; leu for ile, phe, met, or val; val for ile or leu; ile for leu, met, or val; arg for lys; met for phe; tyr for phe or trp; thr for ser; trp for tyr; and phe for tyr.

Functional variant: The term “functional variant,” as used herein, refers to a recombinant enzyme such as a GPPS that comprises a nucleotide and/or amino acid sequence that is altered by one or more nucleotides and/or amino acids compared to the nucleotide and/or amino acid sequences of the parent protein and that is still capable of performing an enzymatic function (e.g., synthesis of GPP) of the parent enzyme. In other words, the modifications in the amino acid and/or nucleotide sequence of the parent enzyme may cause desirable changes in reaction parameters without altering fundamental enzymatic function encoded by the nucleotide sequence or containing the amino acid sequence. The functional variant may have conservative change including nucleotide and amino acid substitutions, additions and deletions. These modifications can be introduced by standard techniques known in the art, such as site-directed mutagenesis and random PCR-mediated mutagenesis, and may comprise natural as well as non-natural nucleotides and amino acids. Also envisioned is the use of amino acid analogs, e.g. amino acids not DNA or RNA encoded in biological systems, and labels such as fluorescent dyes, radioactive elements, electron dense agents, or any other protein modification, now known or later discovered.

Recombinant nucleic acid and recombinant protein: As used herein, a recombinant nucleic acid or protein is a nucleic acid or protein produced by recombinant DNA technology, e.g., as described in Green and Sambrook (2012).

Polypeptide, protein, and peptide: The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, and the like. Modifications also include intra-molecular crosslinking and covalent attachment of various moieties such as lipids, flavin, biotin, polyethylene glycol or derivatives thereof, and the like. In addition, modifications may also include protein cyclization, branching of the amino acid chain, and cross-linking of the protein. Further, amino acids other than the conventional twenty amino acids encoded by genes may also be included in a polypeptide.

The term “protein” or “polypeptide” may also encompass a “purified” polypeptide that is substantially separated from other polypeptides in a cell or organism in which the polypeptide naturally occurs (e.g., 30%, 40%, 50%, 60%, 70%, 80%, 90%, 95%, 96%, 97%, 98%, 99%, 100% free of contaminants).

Primer, probe and oligonucleotide: The terms “primer,” “probe,” and “oligonucleotide” may be used herein interchangeably to refer to a relatively short nucleic acid fragment or sequence. They can be DNA, RNA, or a hybrid thereof, or chemically modified analogs or derivatives thereof. Typically, they are single-stranded. However, they can also be double-stranded having two complementing strands that can be separated apart by denaturation. In certain aspects, they are of a length of from about 8 nucleotides to about 200 nucleotides. In other aspects, they are from about 12 nucleotides to about 100 nucleotides. In additional aspects, they are about 18 to about 50 nucleotides. They can be labeled with detectable markers or modified in any conventional manners for various molecular biological applications.

Vector: As used herein, the term “vector” refers to a nucleic acid molecule capable of transporting another nucleic acid to which it has been linked. One type of vector is an episome, i.e., a nucleic acid capable of extra-chromosomal replication. Various vectors are those capable of autonomous replication and/expression of nucleic acids to which they are linked. Vectors capable of directing the expression of genes to which they are operatively linked are referred to herein as “expression vectors.”

Linker: The term “linker” refers to a short amino acid sequence that separates multiple domains of a polypeptide. In some embodiments, the linker prohibits energetically or structurally unfavorable interactions between the discrete domains.

Cannabinoid: As used herein, the term “cannabinoid” refers to a family of structurally related meroterpenoid molecules, all products of a common biosynthesis pathway.

Terpenoid: As used herein, the term “terpenoid” refers to a family of structurally related organic molecules derived from the 5-carbon compound isoprene, and the isoprene polymers called terpenes.

Codon optimized: As used herein, a recombinant gene is “codon optimized” when its nucleotide sequence is modified to accommodate codon bias of the host organism to improve gene expression and increase translational efficiency of the gene.

Expression cassette: As used herein, an “expression cassette” is a nucleic acid that comprises a gene and a regulatory sequence operatively coupled to the gene such that the promoter drives the expression of the gene in a cell. An example is a gene for an enzyme with a promoter functional in yeast, where the promoter is situated such that the promoter drives the expression of the enzyme in a yeast cell.

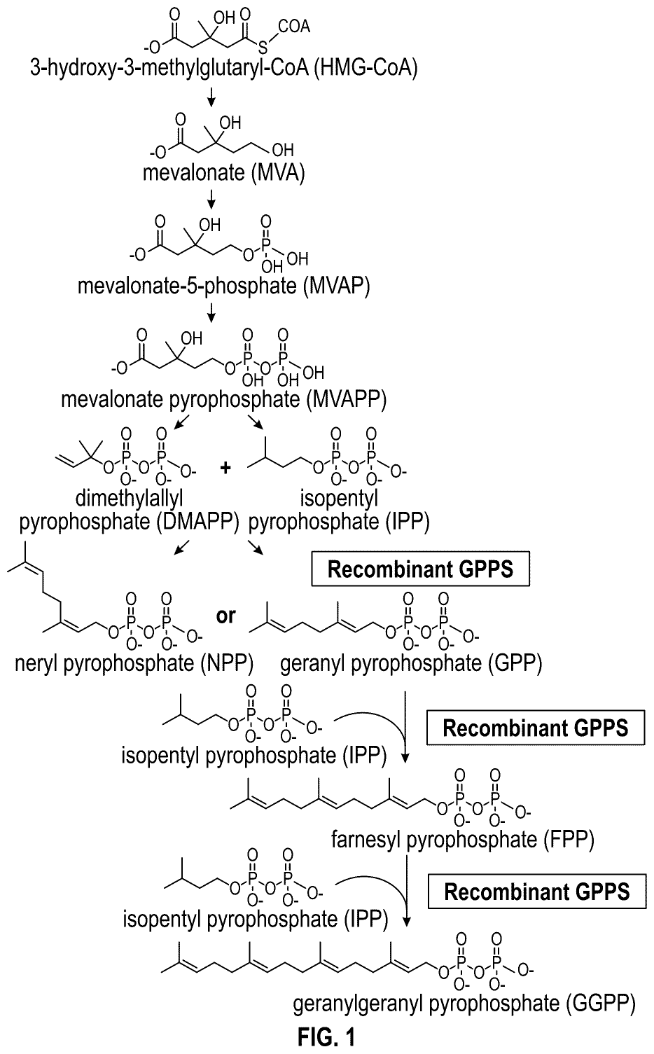

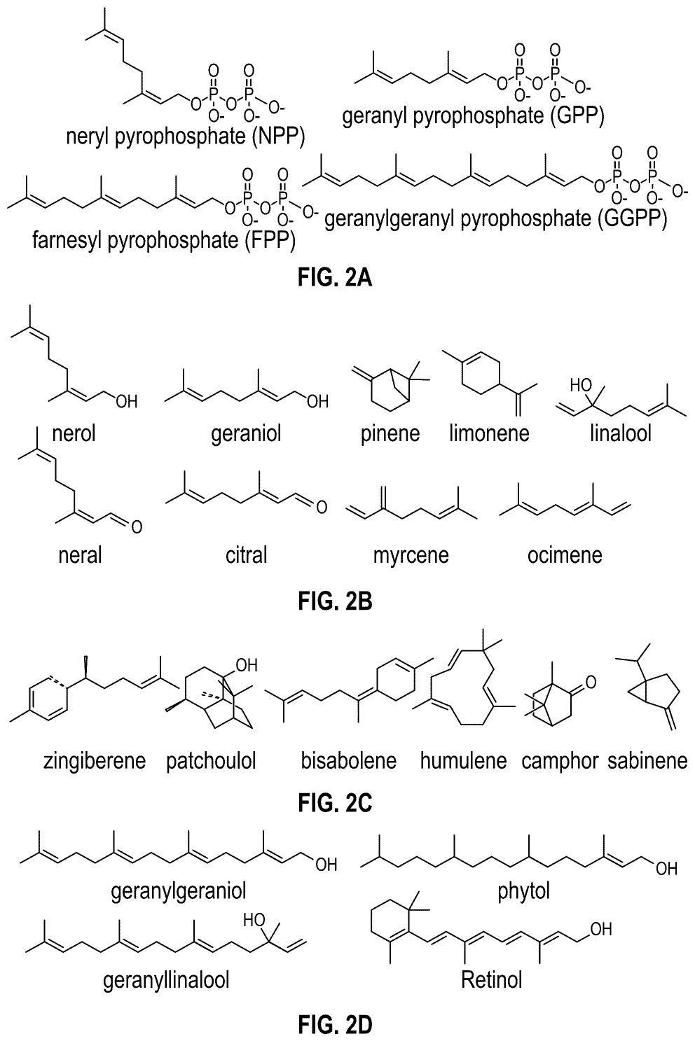

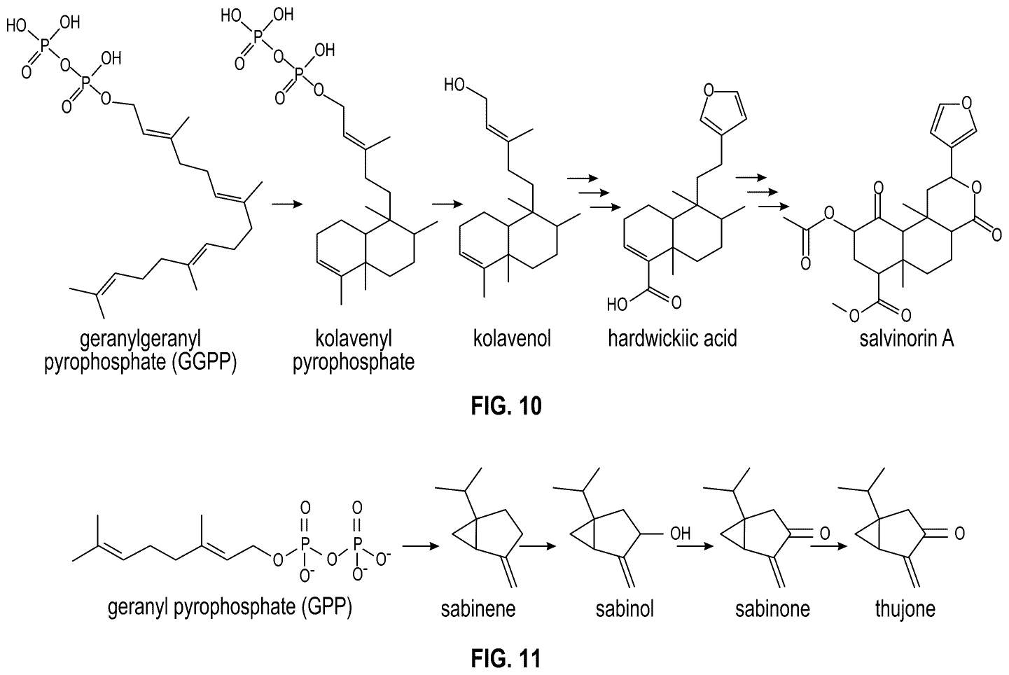

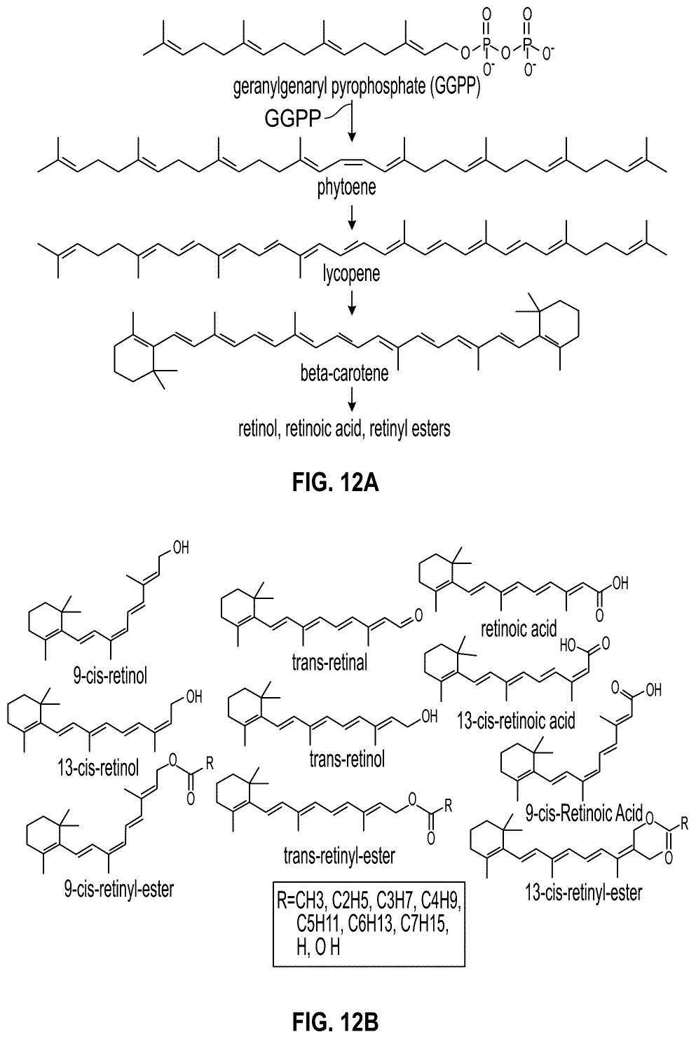

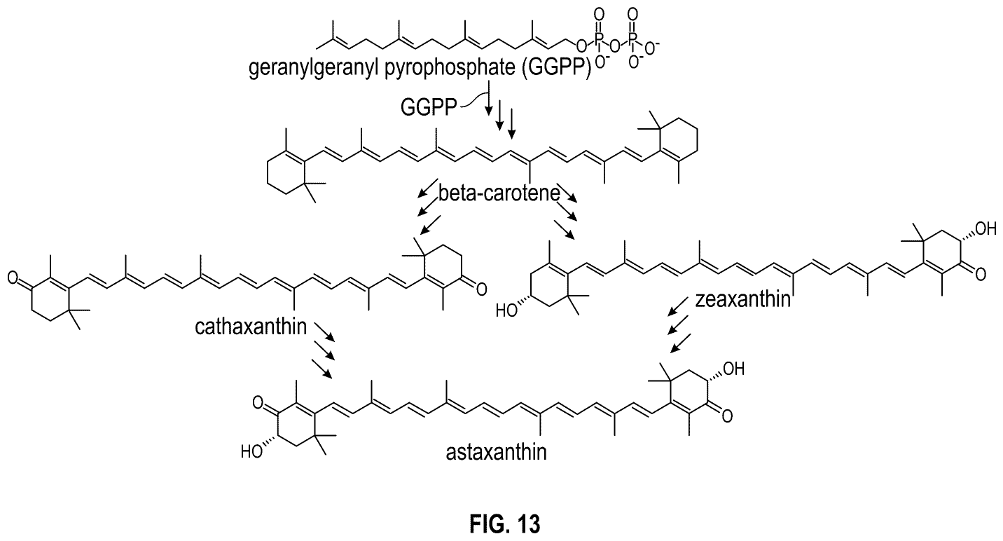

An important precursor molecule in the biosynthesis of cannabinoids and terpenes is geranyl pyrophosphate (GPP), also called geranyl diphosphate (FIG. 1). GPP is made biosynthetically by condensation of two 5-carbon isoprenoids, IPP (isopentenyl pyrophosphate) and DMAPP (dimethyl allylpyrophosphate). The biosynthetic reaction is catalyzed by a GPP synthase or dimethylallyltranstransferase. This reaction can also yield the cis geometric isomer of GPP, neryl pyrophosphate (NPP), also called neryl diphosphate. Further addition of another 5-carbon isoprenoid (IPP) to GPP yields farnesyl pyrophosphate (FPP), also called farnesyl diphosphate. Further addition of another 5-carbon isoprenoid (IPP) to FPP yields geranylgeranyl pyrophosphate (GGPP), also called geranylgeranyl diphosphate (FIG. 1). GPP is thus a key molecule in cannabinoid and other terpenoid pathways. Additional terpenes that can be derived from GPP or GGPP are kolavenol and salvinorin A (FIG. 10); monoterpenes such as thujone (FIG. 11), beta-carotene, retinol, retinoic acid, and retinyl esters (FIGS. 12A and 12B); and diterpenes such as astaxanthin (FIG. 13).

For a diterpenoid product such as the alkaloid salvinorin. GPP is modified by enzymes of the salvinorin biosynthesis pathway to create first, clerodienyl diphosphate or kolavenol diphosphate, as depicted in FIG. 10 (Pelot et al., 2016).

For biosynthesis of the GPP derived terpene thujone, GPP is first converted to sabinene by sabinene synthase (Kshatriya, 2020). See FIG. 11.

Diterpenoids such as carotenoids are derived from GGPP. First, GGPP is converted to phytoene by phytoene synthase, then phytoene to lycopene, beta carotene, canthaxanthin, astaxanthin and derivatives of these molecules (FIGS. 12A, 12B, and 13).

It would therefore be useful to utilize GPP synthase (GPPS) in recombinant systems such as yeast to produce cannabinoids and other terpenoid compounds.

### Nucleic Acids and Polypeptides

Thus, provided is a nucleic acid comprising a recombinant bacterial or archaeal geranyl pyrophosphate synthase (GPPS) gene, codon optimized for production in yeast. Nonlimiting examples of such nucleic acids include GPPS genes having SEQ ID NOs:1-46, encoding proteins having amino acid SEQ ID NOs:47-92, respectively (Table 1). These bacterial GPP synthase (bkGPPS) enzymes and archaeal GPP synthase (rkGPPS) enzymes have the capacity to synthesize GPP, NPP, FPP and/or GGPP in a recombinant host. Because they are codon optimized, they catalyze the production of GPP, NPP, FPP and/or GGPP more efficiently and with higher yield than the naturally occurring enzymes from which they are derived. The codon optimization is specific for a particular host. Additional enzymes may be selected from bacterial and archaeal hosts from a wide variety of habitats in order to match the conditions under which they will be utilized industrially to maximize or maintain enzymatic activity. For example, if the fermentation is to be run at high temperature, it may be beneficial to select a sequence derived from a thermophilic bacterium or archaeon.

TABLE 1

| Shorthand | Codon Optimized | Amino Acid Sequence |
|---|---|---|
| name | Nucleic Acid Sequence | for Isolated Protein |
| bkGPPS1 | Seq. ID NO: 1 | Seq. ID NO: 47 |
| bkGPPS2 | Seq. ID NO: 2 | Seq. ID NO: 48 |
| bkGPPS3 | Seq. ID NO: 3 | Seq. ID NO: 49 |
| bkGPPS4 | Seq. ID NO: 4 | Seq. ID NO: 50 |
| bkGPPS5 | Seq. ID NO: 5 | Seq. ID NO: 51 |
| bkGPPS6 | Seq. ID NO: 6 | Seq. ID NO: 52 |
| bkGPPS7 | Seq. ID NO: 7 | Seq. ID NO: 53 |
| bkGPPS8 | Seq. ID NO: 8 | Seq. ID NO: 54 |
| bkGPPS9 | Seq. ID NO: 9 | Seq. ID NO: 55 |
| bkGPPS10 | Seq. ID NO: 10 | Seq. ID NO: 56 |
| bkGPPS11 | Seq. ID NO: 11 | Seq. ID NO: 57 |
| bkGPPS12 | Seq. ID NO: 12 | Seq. ID NO: 58 |
| bkGPPS13 | Seq. ID NO: 13 | Seq. ID NO: 59 |
| bkGPPS14 | Seq. ID NO: 14 | Seq. ID NO: 60 |
| bkGPPS15 | Seq. ID NO: 15 | Seq. ID NO: 61 |
| bkGPPS16 | Seq. ID NO: 16 | Seq. ID NO: 62 |
| bkGPPS17 | Seq. ID NO: 17 | Seq. ID NO: 63 |
| bkGPPS18 | Seq. ID NO: 18 | Seq. ID NO: 64 |
| bkGPPS19 | Seq. ID NO: 19 | Seq. ID NO: 65 |
| bkGPPS20 | Seq. ID NO: 20 | Seq. ID NO: 66 |
| bkGPPS21 | Seq. ID NO: 21 | Seq. ID NO: 67 |
| bkGPPS22 | Seq. ID NO: 22 | Seq. ID NO: 68 |
| bkGPPS23 | Seq. ID NO: 23 | Seq. ID NO: 69 |
| bkGPPS24 | Seq. ID NO: 24 | Seq. ID NO: 70 |
| rkGPPS1 | Seq. ID NO: 25 | Seq. ID NO: 71 |
| rkGPPS2 | Seq. ID NO: 26 | Seq. ID NO: 72 |
| rkGPPS3 | Seq. ID NO: 27 | Seq. ID NO: 73 |
| rkGPPS4 | Seq. ID NO: 28 | Seq. ID NO: 74 |
| rkGPPS5 | Seq. ID NO: 29 | Seq. ID NO: 75 |
| rkGPPS6 | Seq. ID NO: 30 | Seq. ID NO: 76 |
| rkGPPS7 | Seq. ID NO: 31 | Seq. ID NO: 77 |
| rkGPPS8 | Seq. ID NO: 32 | Seq. ID NO: 78 |
| rkGPPS9 | Seq. ID NO: 33 | Seq. ID NO: 79 |
| rkGPPS10 | Seq. ID NO: 34 | Seq. ID NO: 80 |
| rkGPPS11 | Seq. ID NO: 35 | Seq. ID NO: 81 |
| rkGPPS12 | Seq. ID NO: 36 | Seq. ID NO: 82 |
| rkGPPS13 | Seq. ID NO: 37 | Seq. ID NO: 83 |
| rkGPPS14 | Seq. ID NO: 38 | Seq. ID NO: 84 |
| rkGPPS15 | Seq. ID NO: 39 | Seq. ID NO: 85 |
| rkGPPS16 | Seq. ID NO: 40 | Seq. ID NO: 86 |
| rkGPPS17 | Seq. ID NO: 41 | Seq. ID NO: 87 |
| rkGPPS18 | Seq. ID NO: 42 | Seq. ID NO: 88 |
| rkGPPS19 | Seq. ID NO: 43 | Seq. ID NO: 89 |
| rkGPPS20 | Seq. ID NO: 44 | Seq. ID NO: 90 |
| rkGPPS21 | Seq. ID NO: 45 | Seq. ID NO: 91 |
| rkGPPS22 | Seq. ID NO: 46 | Seq. ID NO: 92 |

The nucleic acid sequences in Table 1 having SEQ ID NOs:1-46 are codon optimized to improve expression using techniques as disclosed in U.S. Pat. No. 10,435,727, which is incorporated herein by reference in its entirety. SEQ ID NOs:1-24 are derived from bacterial GPPS (“bkGPP”) and SEQ ID NOs:25-46 are derived from archaeal GPPS (“rkGPP”).

More specifically, optimized nucleotide sequences are generated based on a number of considerations: (1) For each amino acid of the recombinant polypeptide to be expressed, a codon (triplet of nucleotide bases) is selected based on the frequency of each codon in the Saccharomyces cerevisiae genome; the codon can be chosen to be the most frequent codon or can be selected probabilistically based on the frequencies of all possible codons. (2) In order to prevent DNA cleavage due to a restriction enzyme, certain restriction sites are removed by changing codons that cover those sites. (3) To prevent low-complexity regions, long repeats (sequences of any single base longer than five bases) are modified. (2) and (3) are performed recursively to ensure that codon modification does not lead to additional undesirable sequences. (4) A ribosome binding site is added to the N-terminus. (5) A stop codon is added.

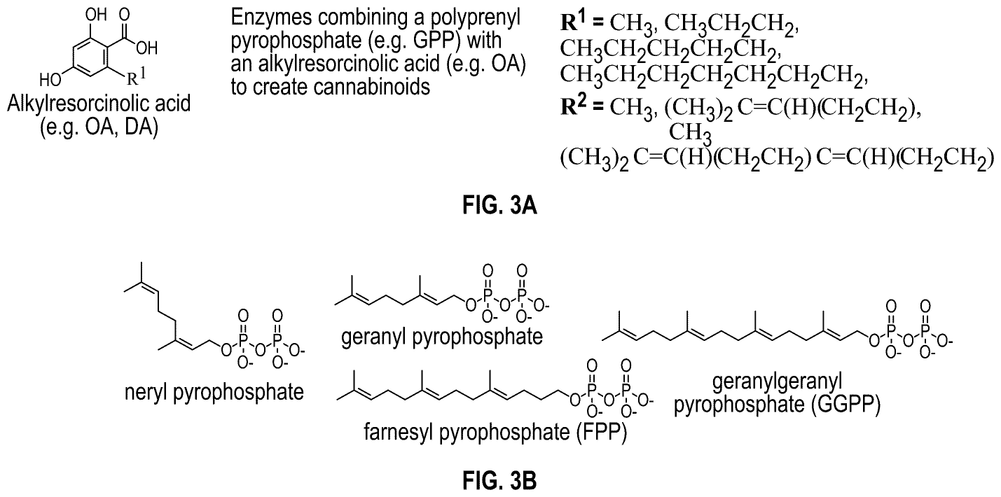

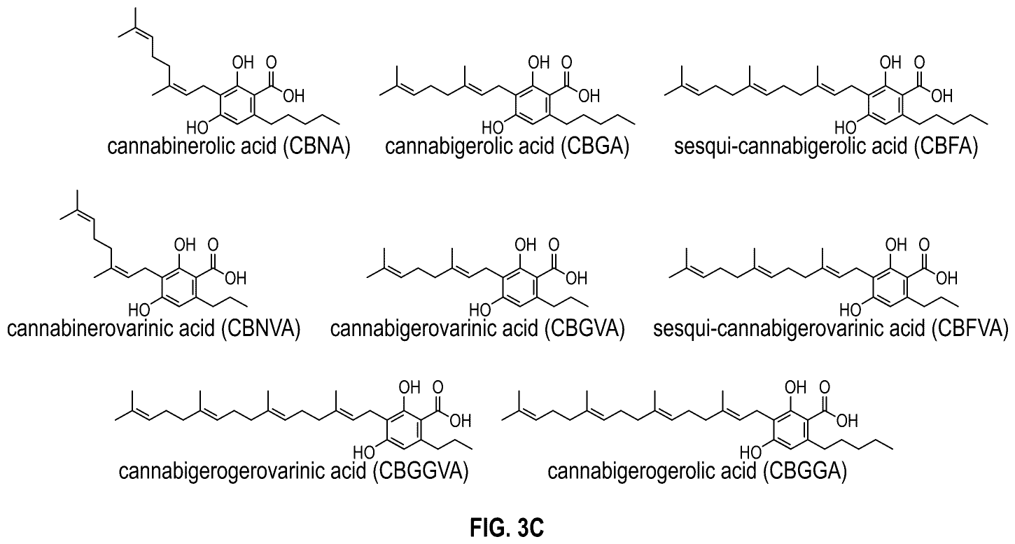

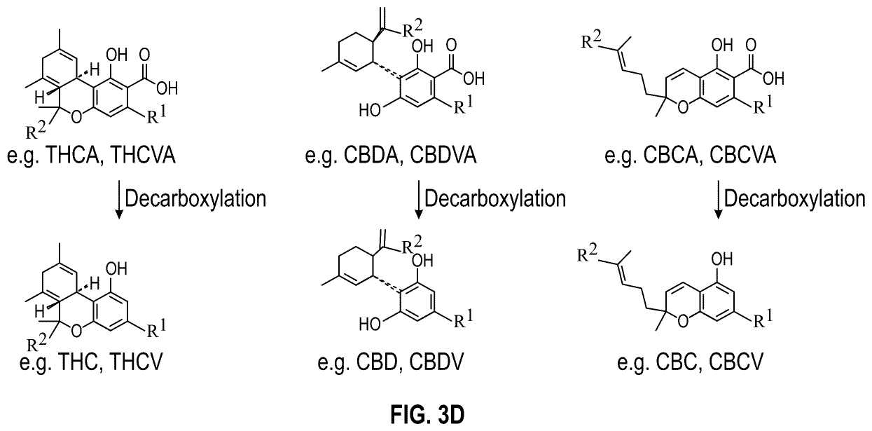

Biosynthesis of sesquiterpenes utilize farnesyl pyrophosphate (FIG. 3) as the starting precursor. Thus, for sesquiterpene biosynthesis, it would be desirable to increase FPP levels, using bacterial or archaeal enzymes that preferentially produce FPP.

Additionally, the class of terpenes known as diterpenes is derived from geranylgeranyl pyrophosphate (FIG. 3). For diterpene biosynthesis, it would be desirable to increase GGPP levels, using bacterial or archaeal enzymes that preferentially produce GGPP.

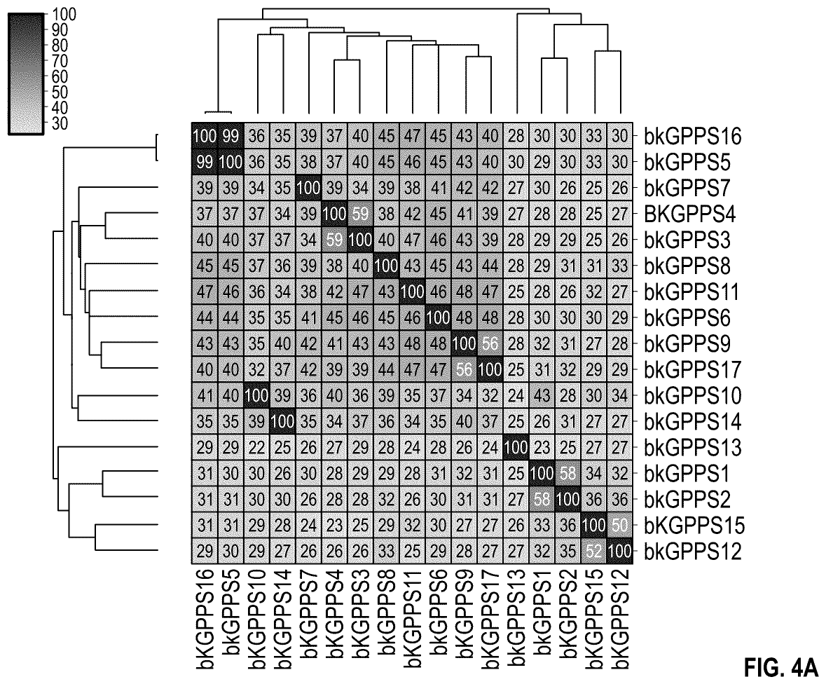

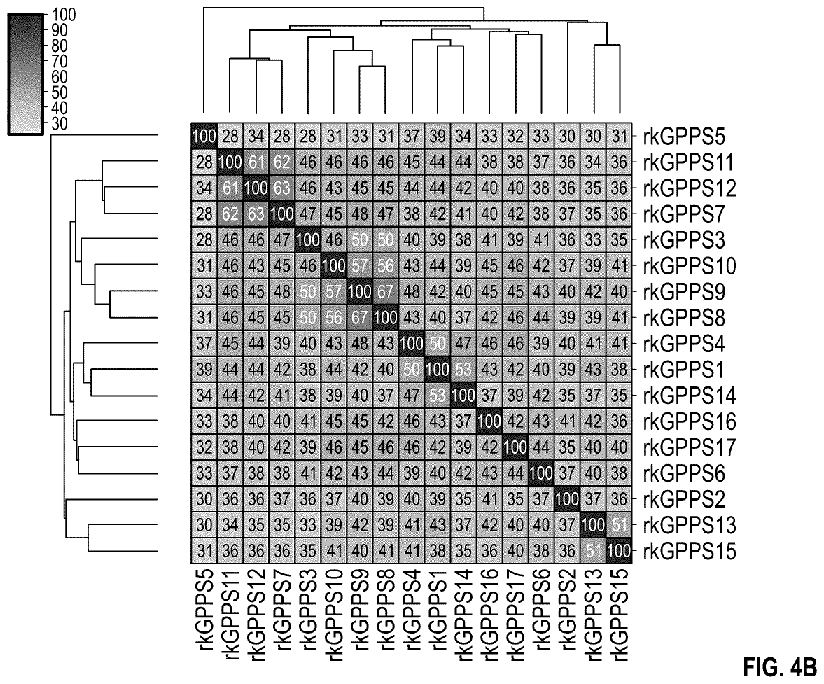

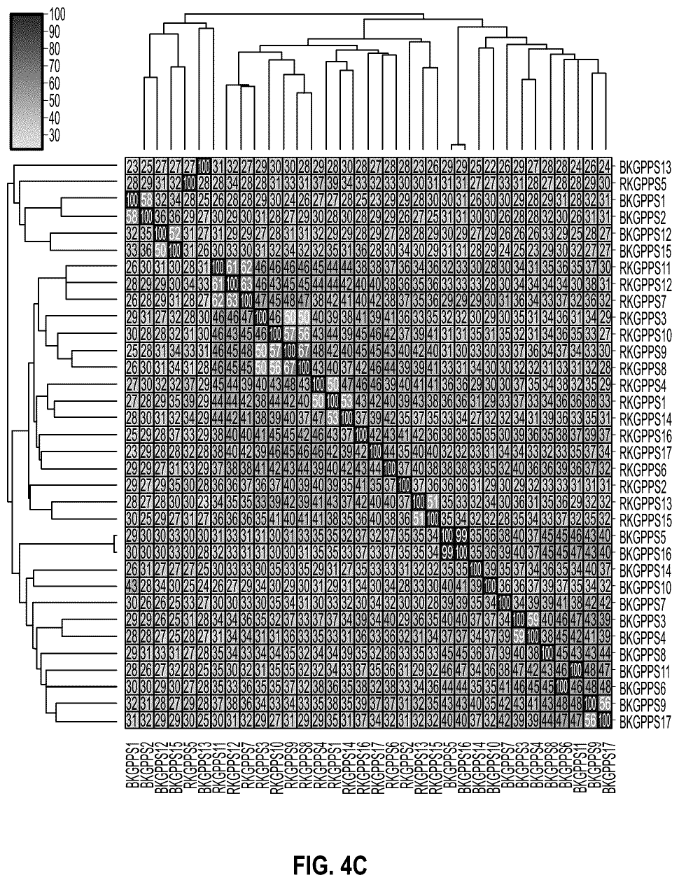

FIGS. 4A, 4B and 4C depict cluster maps comparing A) pairs of bkGPPS enzymes evaluated, B) pairs of rkGPPS enzymes evaluated, and C) bkGPPS and rkGPPS enzymes together. The value in each cell is the percentage of identical residues between each pair of amino acid sequences between the recombinant GPPSs.

In some embodiments, the nucleic acid comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% identical to any one of the thirty-five sequences of SEQ ID NOs:1-46, or its complement, or an RNA equivalent thereof.

In other embodiments, the nucleic acids provided herein encode an enzymatically active GPPS comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity or conservative amino acid substitution to any one of the forty-six sequences of SEQ ID NOs:47-92. These polypeptides are capable of synthesizing GPP, FPP, and/or GGPP.

In some embodiments, the GPPS gene is derived from a bacterium. It is envisioned that a GPPS from any bacterium now known or later discovered can be utilized in the present invention. For example, the bacterium can be from phylum Abditibacteriota, including class Abditibacteria, including order Abditibacteriales; phylum Abyssubacteria or Acidobacteria, including class Acidobacteriia, Blastocatellia, Holophagae, Thermoanaerobaculia, or Vicinamibacteria, including order Acidobacteriales, Bryobacterales, Blastocatellales, Acanthopleuribacterales, Holophagales, Thermotomaculales, Thermoanaerobaculales, or Vicinamibacteraceae; phylum Actinobacteria, including class Acidimicrobiia, Actinobacteria, Actinomarinidae, Coriobacteriia, Nitriliruptoria, Rubrobacteria, or Thermoleophilia, including orders Acidimicrobiales, Acidothermales, Actinomycetales, Actinopolysporales, Bifidobacteriales, Nanopelagicales, Catenulisporales, Corunebacteriales, Cryptosporangiales, Frankiales, Geodermatophilales, Glycomycetales, Jiangellales, Micrococcales, Micromonosporales, Nakamurellales, Propionibacteriales, Pseudonocardiales, Sporichthyales, Streptomycetales, Streptosporangiales, Actinomarinales, Coriobacteriales, Eggerthellales, Egibacterales, Egicoccales, Euzebyales, Nitriliruptorales, Gaiellales, Rubrobacterales, Solirubrobacterales, or Thermoleophilales; phylum Aquificae, including class Aquificae, including order Aquificales or Desulfurobacteriales; phylum Armatimonadetes, including class Armatimonadia, including order Armatimonadales, Capsulimonadales, Chthonomonadetes, Chthonomonadales, Fimbriimonadia, or Fimbriimonadales; phylum Aureabacteria or Bacteroidetes, including class Armatimonadia, Bacteroidia, Chitinophagia, Cytophagia, Flavobacteria, Saprospiria or Sphingobacteriia, including order B acteroidales, Marinilabiliales, Chitinophag ales, Cytophag ales, Flavobacteriales, Saprospirales, or Sphingopacteriales; phylum Balneolaeota, Caldiserica, Calditrichaeota, or Chlamydiae, including class B alneolia, Caldisericia, Calditrichae, or Chlamydia, including order Balneolales, C aldiseric ale s, Calditrichales, Anoxychlamydiales, Chlamydiales, or Parachlamydiales; phylum Chlorobi or Chloroflexi, including class Chlorobia, Anaerolineae, Ardenticatenia, Caldilineae, Thermofonsia, Chloroflexia, Dehalococcoidia, Ktedonobacteria, Tepidiformia, Thermoflexia, Thermomicrobia, or Sphaerobacteridae, including order Chlorobiales, Anaerolineales, Ardenticatenales, Caldilineales, Chloroflexales, Herpetosiphonales, Kallotenuales, Dehalococcoidales, Dehalogenimonas, Ktedonobacterales, Thermogemmatisporales, Tepidiformales, Thermoflexales, Thermomicrobiales, or Sphaerobacterales; phylum Chrysiogenetes, Cloacimonetes, Coprothermobacterota, Cryosericota, or Cyanobacteria, including class Chrysiogenetes, Coprothermobacteria, Gloeobacteria, or Oscillatoriophycideae, including order Chrysiogenales, Coprothermobacterales, Chroococcidiopsidales, Gloeoemargaritales, Nostocales, Pleurocapsales, Spirulinales, Synechococcales, Gloeobacterales, Chroococcales, or Oscillatoriales; phyla: Eferribacteres, Deinococcus-thermus, Dictyoglomi, Dormibacteraeota, Elusimicrobia, Eremiobacteraeota, Fermentibacteria, or Fibrobacteres, including class Deferribacteres, Deinococci, Dictyoglomia, Elusimicrobia, Endomicrobia, Chitinispirillia, Chitinivibrionia, or Fibrobacteria, including order Deferribacterales, Deinococcales, Thermales, Dictyoglomales, Elusimicrobiales, Endomicrobiales, Chitinspirillales, Chitinvibrionales, Fibrobacterales, or Fibromonadales; phylum Firmicutes, Fusobacteria, Gemmatimonadetes, or Hydrogenedentes, including class Bacilli, Clostridia, Erysipelotrichia, Limnochordia, Negativicutes, Thermolithobacteria, Tissierellia, Fusobacteriia, Gemmatimonadetes, Longimicrobia, including order Bacillales, Lactobacillales, Borkfalkiales, Clostridiales, Halanaerobiales, Natranaerobiales, Thermoanaerobacterales, Erysipelotrichales, Limnochordales, Acidaminococcales, Selenomonadales, Veillonellales, Thermolithobacterales, Tissierellales, Fusobacteriales, Gemmatimonadales, or Longimicrobia; phylum Hydrogenedentes, Ignavibacteriae, Kapabacteria, Kiritimatiellaeota, Krumholzibacteriota, Kryptonia, Latescibacteria, LCP-89, Lentisphaerae, Margulisbacteria, Marinimicrobia, Melainabacteria, Nitrospinae, or Omnitrophica, including class Ignavibacteria, Kiritimatiellae, Krumholzibacteria, Lentisphaeria, Oligosphaeria, or Nitrospinae, including order Ignavibacteriales, Kiritimatiellales, Krumholzibacteriales, Lentisphaerales, Victivallales, Oligosphaerales, or Nitrospinia; phylum Omnitrophica or Planctomycetes, including class Brocadiae, Phycisphaerae, Planctomycetia, or Phycisphaerales, including order Sedimentisphaerales, Tepidisphaerales, Gemmatales, Isosphaerales, Pirellulales, or Planctomycetales; phylum Proteobacteria including class Acidithiobacillia, Alphaproteobacteria, Betaproteobacteria, Lambdaproteobacteria, Muproteobacteria, Deltaproteobacteria, Epsilonproteobacteria, Gammaproteobacteria, Hydrogenophilalia, Oligoflexia, or Zetaproteobacteria, including order Acidithiobacillales, Caulobacterales, Emcibacterales, Holosporales, lodidimonadales, Kiloniellales, Kopriimonadales, Kordiimonadales, Magnetococcales, Micropepsales, Minwuiales, Parvularculales, Pelagibacterales, Rhizobiales, Rhodobacterales, Rhodospirillales, Rhodothalas siales, Rickettsiales, Sneathiellales, Sphingomonadales, Burkholderiales, Ferritrophicales, Ferrovales, Neis seriales, Nitrosomonadales, Procabacteriales, Rhodocyclales, Bradymonadales, Acidulodesulfobacterales, Desulfarculales, Desulfobacterales, Desulfovibrionales, Desulfurellales, Desulfuromonadales, Myxococcales, Syntrophobacterales, Campylobacterales, Nautiliales, Acidiferrobacterales, Aeromonadales, Alteromonadales, Arenicellales, Cardiobacteriales, Cellvibrionales, Chromatiales, Enterobacterales, Immundisolibacterales, Legionellales, Methylococcales, Nevskiales, Oceanospirillales, Orbales, Pasteurellales Pseudomonadales, Salinisphaerales, Thiotrichales, Vibrionales, Xanthomonadales, Hydrogenophilales, Bacteriovoracales, Bdellovibrionales, Oligoflexales, Silvanigrellales, or Mariprofundales; phylum Rhodothermaeota, Saganbacteria, Sericytochromatia, Spirochaetes, Synergistetes, Tectomicrobia, or Tenericutes, including class Rhodothermia, Spirochaetia, Synergistia, Izimaplasma, or Mollicutes, including order Rhodothermales, Brachyspirales, Brevinematales, Leptospirales, Spirochaetales, Synergistales, Acholeplasmatales, Anaeroplasmatales, Entomoplasmatales, or Mycoplasmatales; phylum Thermodesulfobacteria, Thermotogae, Verrucomicrobia, or Zixibacteria, including class Thermodesulfobacteria, Thermotogae, Methylacidiphilae, Opitutae, Spartobacteria, or Verrucomicrobiae, including order Thermodesulfobacteriales, Kosmotogales, Mesoaciditogales, Petrotogales, Thermotogales, Methylacidiphilales, Opitutales, Puniceicoccales, Xiphinematobacter, Chthoniobacterales, Terrimicrobium, or Verrucomicrobiales.

In other embodiments, the GPPS gene is derived from an archaeon. It is envisioned that a GPPS from any archaeon now known or later discovered can be utilized in the present invention. For example, the bacterium can be from phylum Euryarchaeota, including class Archaeoglobi, Hadesarchaea, Halobacteria, Methanobacteria, Methanococci, Methanofastidiosa, Methanomicrobia, Methanopyri, Nanohaloarchaea, Theionarchaea, Thermococci, or Thermoplasmata, including order Archaeoglobales, Hadesarchaeales, Halobacteriales, Methanobacteriales, Methanococcales, Methanocellales, Methanomicrobiales, Methanophagales, Methanosarcinales, Methanopyrales, Thermococcales, Methanomas siliicoccales, Thermoplasmatales, or Nanoarchaeales; DPANN superphylum, including subphyla Aenigmarcheota, Altiarchaeota, Diapherotrites, Micrarchaeota, Nanoarchaeota, Pacearchaeota, Parvarchaeota, or Woesearchaeota; TACK superphylum, including subphylum Korarchaeota, Crenarchaeota, Aigarchaeota, Geoarchaeota, Thaumarchaeota, or Bathyarchaeota; Asgard superphylum including subphylium Odinarchaeota, Thorarchaeota, Lokiarchaeota, Helarchaeota, or Heimdallarchaeota.

The nucleic acids of the present invention can further comprise additional nucleotide sequences or other molecules. In some embodiments, the additional sequences encode additional amino acids present when the nucleic acid is translated, encoding, for example, an additional protein domain, with or without a linker sequence, creating a fusion protein. Other examples are localization sequences, i.e., signals directing the localization of the folded protein to a specific subcellular compartment or membrane.

In some embodiments, any of the codon optimized nucleic acids having sequences SEQ ID NOs:1-46 are have, at the 5′ end, a nucleic acid encoding codon optimized cofolding peptides to create a fusion protein, e.g., having SEQ ID NOs:93-97 (Table 2), joining the sequences together to form a fusion polypeptide, e.g., having the amino acid sequence of SEQ ID NO:98-102 fused at the N terminus of any of the polypeptides having SEQ ID NO:47-92, generating recombinant fusion polypeptides.

TABLE 2

| | Codon Optimized | Amino Acid Sequence |
|---|---|---|
| NAME | Nucleic Acid Sequence | for Isolated Protein |
| MBP | Seq. ID NO: 93 | Seq. ID NO: 98 |
| VEN | Seq. ID NO: 94 | Seq. ID NO: 99 |
| MST | Seq. ID NO: 95 | Seq. ID NO: 100 |
| OSP | Seq. ID NO: 96 | Seq. ID NO: 101 |
| OLE | Seq. ID NO: 97 | Seq. ID NO: 102 |

Other additional amino acids that can be added to the GPPS of the present invention include various yeast protein tags and modifiers. See e.g. http://parts.igem.org/Yeast.

In other embodiments, the nucleic acid comprises additional nucleotide sequences that are not translated. Examples include promoters, terminators, barcodes, Kozak sequences, targeting sequences, and enhancer elements. Particularly useful here are promoters that are functional in yeast.

Expression of a GPPS gene is determined by the promoter controlling the gene. In order for a gene to be expressed, a promoter must be present within 1,000 nucleotides upstream of the GPPS gene. A gene is generally cloned under the control of a desired promoter. The promoter regulates the amount of GPPS enzyme expressed in the cell and also the timing of expression, or expression in response to external factors such as sugar source.

Any promoter now known or later discovered can be utilized to drive the expression of the GPPS genes described herein. See e.g. http://parts.igem.org/Yeast for a listing of various yeast promoters. Exemplary promoters listed in Table 3 below drive strong expression, constant gene expression, medium or weak gene expression, or inducible gene expression. Inducible or repressible gene expression is dependent on the presence or absence of a certain molecule. For example, the GAL1, GAL7, and GAL10 promoters are activated by the presence of the sugar galactose and repressed by the presence of the sugar glucose. The HO promoter is active and drives gene expression only in the presence of the alpha factor peptide. The HXT1 promoter is activated by the presence of glucose while the ADH2 promoter is repressed by the presence of glucose.

TABLE 3
Exemplary yeast promoters

| | Medium and weak | |
|---|---|---|
| Strong constitutive | constitutive | Inducible/repressible |
| promoters | promoters | promoters |
| TEF1 | STE2 | GAL1 |
| PGK1 | TPI1 | GAL7 |
| PGI1 | PYK1 | GAL10 |
| TDH3 | | HO |
| | | HXT1 |
| | | ADH2 |

In various embodiments, the nucleic acid is in a yeast expression cassette. Any yeast expression cassette capable of expressing GPPS in a yeast cell can be utilized. In some embodiments, the expression cassette consists of a nucleic acid encoding a GPPS with a promoter. Additional regulatory elements can also be present in the expression cassette, including restriction enzyme cleavage sites, antibiotic resistance genes, integration sites, auxotrophic selection markers, origins of replication, and degrons.

The expression cassette can be present in a vector that, when transformed into a host cell, either integrates into chromosomal DNA or remains episomal in the host cell. Such vectors are well-known in the art. See e.g. http://parts.igem.org/Yeast for a listing of various yeast vectors.

A nonlimiting example of a yeast vector is a yeast episomal plasmid (YEp) that contains the pBluescript II SK(+) phagemid backbone, an auxotrophic selectable marker, yeast and bacterial origins of replication and multiple cloning sites enabling gene cloning under a suitable promoter (see Table 3). Other exemplary vectors include pRS series plasmids.

### Host Cells

The present invention is also directed to genetically engineered host cells that comprise the above-described nucleic acids. Such cells may be, e.g., any species of filamentous fungus, including but not limited to any species of Aspergillus, which have been genetically altered to produce precursor molecules, intermediate molecules, or cannabinoid molecules. Host cells may also be any species of bacteria, including but not limited to Escherichia, Corynebacterium, Caulobacter, Pseudomonas, Streptomyces, Bacillus, or Lactobacillus.

In some embodiments, the genetically engineered host cell is a yeast cell, which may comprise any of the above-described expression cassettes, and capable of expressing a GPPS comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity or conservative amino acid substitutions to any one of the thirty-four sequences of SEQ ID NOs:47-92.

Any yeast cell capable of being genetically engineered can be utilized in these embodiments. Nonlimiting examples of such yeast cells include species of Saccharomyces, Candida, Pichia, Schizosaccharomyces, Scheffersomyces, Blakeslea, Rhodotorula, or Yarrowia. These cells can achieve gene expression controlled by inducible promoter systems; natural or induced mutagenesis, recombination, and/or shuffling of genes, pathways, and whole cells performed sequentially or in cycles; overexpression and/or deletion of single or multiple genes and reducing or eliminating parasitic side pathways that reduce precursor concentration.

The host cells of the recombinant organism are engineered to produce any or all precursor molecules necessary for the biosynthesis of cannabinoids, including but not limited to olivetolic acid (OA), olivetol (OL), FPP and GPP, hexanoic acid and hexanoyl-CoA, malonic acid and malonyl-CoA, dimethylallylpyrophosphate (DMAPP) and isopentenylpyrophosphate (IPP) as disclosed in U.S. Pat. No. 10,435,727.

Construction of Saccharomyces cerevisiae strains expressing bacterial or archaeal GPPS enzymes to produce GPP, NPP, FPP, and/or GGPP for cannabinoid and/or terpene production, such as CBGA or geraniol, is carried out via expression of a GPPS gene which encodes for an enzyme with GPPS activity such as the archaeal (rkGPPS) and bacterial (bkGPPS) genes and proteins listed in Table 1. The GPPS gene can be cloned into vectors with the proper regulatory elements for gene expression (e.g. promoter, terminator) and the derived plasmid can be confirmed by DNA sequencing. As an alternative to expression from an episomal plasmid, the GPPS gene may be inserted into the recombinant host genome. Integration may be achieved by a single or double cross-over insertion event of a plasmid, or by nuclease based genome editing methods, as are known in the art e.g. CRISPR, TALEN and ZFR. Strains with the integrated gene can be screened by rescue of auxotrophy and genome sequencing. See, e.g., Green and Sambrook (2012)

In some embodiments, the recombinant cell further comprises a second recombinant nucleic acid that encodes a second enzyme in a terpenoid biosynthetic pathway. In some of these embodiments, the yeast cell is capable of expressing the second enzyme.

The second enzyme in these embodiments can encode any enzyme in the terpenoid biosynthetic pathway. In some embodiments, the second enzyme catalyzes synthesis of a compound that immediately precedes or is immediately after a product of the GPPS in the terpenoid biosynthetic pathway.

The recombinant cell can further comprise a third, fourth, etc. recombinant nucleic acid in the terpenoid biosynthetic pathway so that the cell can process a compound through at least three, four, five, etc. steps in the terpenoid biosynthetic pathway.

In some of these embodiments, the terpenoid biosynthetic pathway is not a cannabinoid biosynthetic pathway. In these embodiments, the recombinant cell can co-express genes for downstream terpenoid synthesis (reviewed in Davis and Croteau, 2000) such as cyclases, thiolases, desaturases, hydroxylases, hydrolases, oxidoreductases, and P450s, to produce monoterpenoids including but not limited to: 3-carene, ascaridole, bornane, borneol, camphene, camphor, camphorquinone, carvacrol, carveol, carvone, carvonic acid, chrysanthemic acid, chrysanthenone, citral, citronellal, citronellol, cuminaldehyde, p-cymene, cymenes, epomediol, eucalyptol, fenchol, fenchone, geranic acid, geraniol, geranyl acetate, geranyl pyrophosphate, grandisol, grapefruit mercaptan, halomon, hinokitiol, hydroxycitronellal, 8-hydroxygeraniol, incarvillateine, (s)-ipsdienol, jasmolone, lavandulol, lavandulyl acetate, levoverbenone, limonene, linalool, linalyl acetate, lineatin, p-menthane-3,8-diol, menthofuran, menthol, menthone, menthoxypropanediol, menthyl acetate, 2-methylisoborneol, myrcene, myrcenol, nerol, nerolic acid, ocimene, 8-oxogeranial, paramenthane hydroperoxide, perilla ketone, perillaldehyde, perillartine, perillene, phellandrene, picrocrocin, pinene, alpha-pinene, beta-pinene, piperitone, pulegone, rhodinol, rose oxide, sabinene, safranal, sobrerol, terpinen-4-ol, terpinene, terpineol, thujaplicin, thujene, thujone, thymol, thymoquinone, umbellulone, verbenol, verbenone, and wine lactone.

In other embodiments, the recombinant cell can also co-express genes for downstream terpenoid synthesis to produce sesquiterpenoids including but not limited to: abscisic acid, amorpha-4,11-diene, aristolochene, artemether, artemotil, artesunate, bergamotene, bisabolene, bisabolol, bisacurone, botrydial, cadalene, cadinene, alpha-cadinol, delta-cadinol, capnellene, capsidiol, carotol, caryophyllene, cedrene, cedrol, copaene, cubebene, cubebol, curdione, curzerene, curzerenone, dictyophorine, drimane, elemene, farnesene, farnesol, farnesyl pyrophosphate, germacrene, germacrone, guaiazulene, guaiene, guaiol, gyrinal, hernandulcin, humulene, indometacin farnesil, ionone, isocomene, juvabione, khusimol, koningic acid, ledol, longifolene, matricin, mutisianthol, nardosinone, nerolidol, nootkatone, norpatchoulenol, onchidal, patchoulol, periplanone b, petasin, phaseic acid, polygodial, rishitin, α-santalol, β-santalol, santonic acid, selinene, spathulenol, thujopsene, tripfordine, triptofordin c-2, valencene, velleral, verrucarin a, vetivazulene, α-vetivone, zingiberene.

In further embodiments, the recombinant cell can also co-express genes for downstream terpenoid synthesis to produce diterpenoids including but not limited to: abietane, abietic acid, ailanthone, andrographolide, aphidicolin, beta-araneosene, bipinnatin j, cafestol, cannabigerolic acid, carnosic acid, carnosol, cembratrienol, cembrene a, clerodane diterpene, crotogoudin, 10-deacetylbaccatin, elisabethatriene, erinacine, ferruginol, fichtelite, forskolin, galanolactone, geranylgeraniol, geranylgeranyl pyrophosphate, gibberellin, ginkgolide, grayanotoxin, guanacastepene a, incensole, ingenol mebutate, isocupressic acid, isophytol, isopimaric acid, isotuberculosinol, kahweol, labdane, lagochilin, laurenene, levopimaric acid, menatetrenone, mezerein, momilactone b, neotripterifordin, 18-norabietane, paxilline, phorbol, phorbol 12,13-dibutyrate, phorbol esters, phyllocladane, phytane, phytanic acid, phytol, phytomenadione, pimaric acid, pristane, pristanic acid, prostratin, pseudopterosin a, retinol, salvinorin, saudin, sclarene, sclareol, shortolide a, simonellite, stemarene, stemodene, steviol, taxadiene, taxagifine, taxamairin, taxodone, tenuifolin, 12-o-tetradecanoylphorbol-13-acetate, tigilanol tiglate, totarol, tricholomalide, tripchlorolide, tripdiolide, triptolide, triptolidenol.

In further embodiments, the recombinant cell can also co-express genes for downstream terpenoid modification to produce terpenoid derivatives including but not limited to: cholesterol, steroid hormones and analogs, heme, antioxidants such as carotenoids and quinones.

In specific embodiments, the recombinant cell is capable of producing nerol, geraniol, pinene, limonene, linalool, neral, citral, myrcene, ocimene, zingiberene, patchoulol, bisabolene, humulene, camphor, sabinene, geranylgeraniol, phytol, geranyllinalool, retinol, or any combination thereof.

The production of specific terpenes in recombinant cells can be enhanced by the use of specific recombinant GPPSs that preferentially produces geranyl pyrophosphate (GPP) or farnesyl pyrophosphate (FPP) or geranylgeranyl pyrophosphate (GGPP). For example, to enhance production of a monoterpene, the use of a GPPS that preferentially produces geranyl pyrophosphate (GPP) over farnesyl pyrophosphate (FPP) or geranylgeranyl pyrophosphate (GGPP) is beneficial. Similarly, to enhance production of a sesquiterpene, the use of a GPPS that preferentially produces FPP over GPP or GGPP is beneficial. Also, to enhance production of a diterpene, the use of a GPPS that preferentially produces GGPP over GPP or FPP is beneficial.

In various embodiments, the terpenoid biosynthetic pathway engineered in the recombinant host cell is a cannabinoid biosynthetic pathway. In these embodiments, the cell is capable of producing cannabigerolic acid (CBGA), cannabidiolic acid (CBDA), cannabichromenic acid (CBCA), cannabinerolic acid (CBNA), cannabigerolic acid (CBGA), cannabinerovarinic acid (CBNVA), cannabigerophorolic acid (CB GPA), cannabigerovarinic acid (CBGVA), cannabigerogerovarinic acid (CBGGVA), tetrahydrocannabinolic acid (THCA), cannabinerovarinic acid (CBNVA), sesquicannabigerol (CBF), cannabigerogerol (CBGG), sesqui-cannabigerolic acid (CBFA), cannabigerogerolic acid (CBGGA), sesquicannabigerolic acid (CBFA), sesquicannabidiolic acid (CBDFA), sesquiTHCA (THCFA), sesqui-cannabigerovarinic acid (CBFVA), sesquiCBCA (CBCFA), sesquiCBGPA (CBFPA) or any combination thereof.

To enhance production of a cannabinoid, the use of a GPPS that preferentially produces GPP over FPP is beneficial.

### Methods of Producing Terpenes

The present invention is also directed to a method of producing a terpene in a yeast. The method comprises incubating any of the recombinant yeast cells described above in a manner sufficient to produce the terpene.

In some embodiments, a mixture of different archaeal GPPS (rkGPPS) genes are expressed, a mixture of different bacterial GPPS (bkGPPS) genes are expressed, or a mixture of rkGPPS and bkGPPS are expressed in a modified strain. GPPS genes, such as those listed in Table 1, are synthesized using DNA synthesis techniques known in the art. The rkGPPS and bkGPPS genes can also be expressed in combination with known fungal GPPSes, such as Erg20 and the Erg20 mutants, and other fungal GPPSes (Genbank Accession Identification numbers: AFC92798.1, OBZ88092.1, AMM73096.1, EMS20556.1, CDR39302.1, ATB19148.1, AAY33922.1, ALK24263.1, ALK24264.1). Wild type ERG20 has the following corresponding GenBank Accession Identification Number: CAA89462.1. Certain point mutations in ERG20 have been shown to change product specificity. Examples include: any combination of A99 to C, I, F or W, and F96W and N127W as reported in Ignea (2014), mutation of A99 to any residue as reported in Rubat (2017) and mutation of K197 to any residue as reported in Fischer (2011) especially K197E and K197G. The optimized genes can be cloned into vectors with the proper regulatory elements for gene expression (e.g. promoter and terminator) and the derived plasmid can be confirmed by DNA sequencing. As an alternative to expression from an episomal plasmid, the optimized prenyltransferase genes are inserted into the recombinant host genome. Integration is achieved by a single cross-over insertion event of the plasmids. Strains with the integrated genes can be screened by rescue of auxotrophy and genome sequencing.

In some embodiments, a monoterpene is produced. In some of these embodiments, a recombinant GPPS that preferentially produces GPP over FPP or GGPP is utilized. In other embodiments, a sesquiterpene is produced. In some of these embodiments, a recombinant GPPS that preferentially produces FPP over GPP or GGPP is utilized. In additional embodiments, a diterpene is produced. In some of these embodiments, a recombinant GPPS that preferentially produces GGPP over GPP and FPP is utilized.

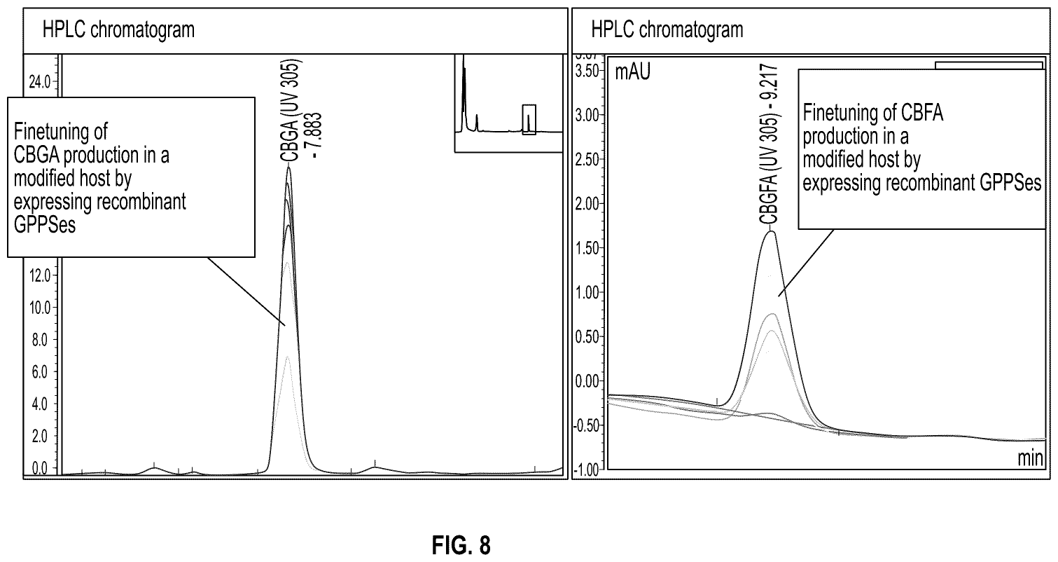

Depending on the desired target molecule, it may be beneficial to selectively produce or increase GPP, FPP, or GGPP levels or modulate the ratio of GPP:FPP, GPP:GGPP, or FPP:GGPP to selectively obtain a desired end product (see FIGS. 1 and 8). To that end, the GPPS enzymes herein disclosed comprise a system that allows finetuning of the mevalonate pathway flux to produce the precursor of choice for production of a particular cannabinoid or terpene.

For the biosynthesis of phytocannabinoids such as CBG, CBD, CBC, and THC, the presence of farnesyl pyrophosphate (FPP) is undesirable as it may be combined with the prenyl acceptor molecule in place of GPP, yielding an undesirable sesquicannabinoid byproduct. To maximize production of cannabinoids such as THC and CBD, the concentration of GPP should be maximized and the concentration of FPP minimized. The pathway making both GPP and FPP in fungi is the mevalonate pathway, whose end product is ergosterol. In this pathway, GPP is the immediate precursor of FPP. However, GPP and FPP are synthesized by the same enzyme in yeast, Erg20, making it challenging to manipulate the Erg20 enzyme to produce predominantly GPP or predominantly FPP.

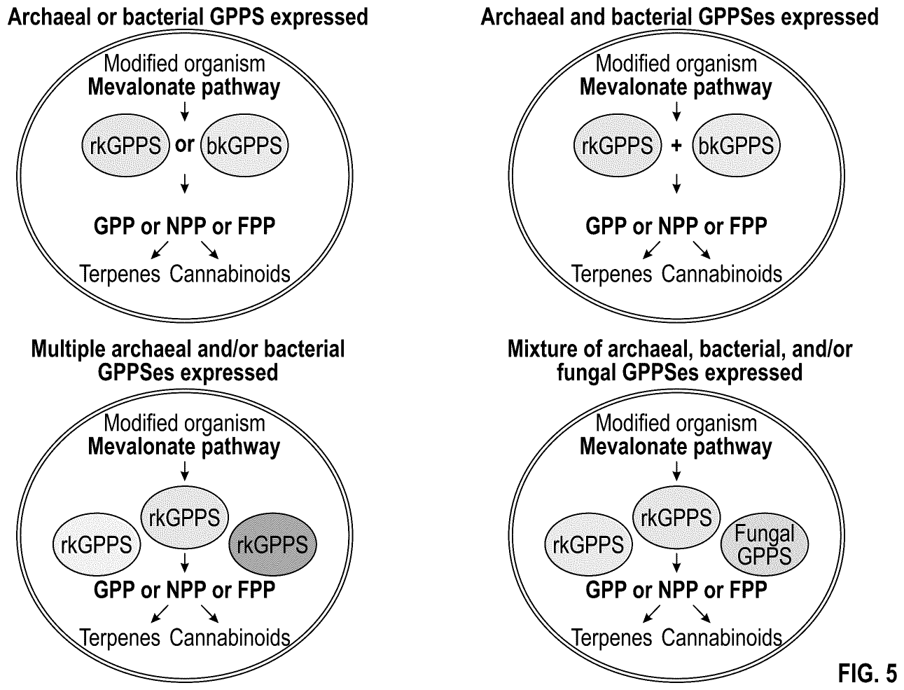

In yeast, some mutant alleles of the ERG20 gene use steric hindrance in the prenyl donor binding site of the enzymes to bias the synthase towards producing more GPP than FPP. The endogenous copy or copies of ERG20 can be replaced entirely by an engineered version of ERG20 to remove or greatly reduce the endogenous capacity to make FPP. While protein engineering approaches have been very successful in conferring specificity for GPP production over FPP, some of these mutations negatively affect the catalytic efficiency and catalytic rate of the enzyme (Ignea, 2013 and Rubat, 2017). Although not as catalytically efficient as the wild type enzyme, the engineered yeast enzyme can be used in combination with bacterial or archaeal GPP synthases disclosed herein to increase the concentration of GPP while maintaining specificity (see FIG. 5).

Conversely, FPP pools in an engineered host cell can be increased by certain other mutations of the endogenous Erg20. The engineered Erg20 fungal GPPS may be used in combination with a bacterial or archaeal enzyme that preferentially synthesizes FPP (FIG. 5).

Pathways for GPP biosynthesis differ in other kingdoms. Bacteria use the methyl erythritol phosphate pathway, using entirely different biosynthetic enzymes and intermediates to make GPP. Archaea have a modified form of the mevalonate pathway (Vinokur, 2014). This presents the possibility that GPP synthase homologs derived from bacteria and archaea may have different GPP:FPP product ratios. Although they may also make FPP, some bacterial and archaeal enzymes may have an advantage for GPP production, while others are more prone to generate FPP.

Thus, the set of recombinant heterologous enzymes disclosed offers a variety of options for constructing a modified host system biased either towards the production of FPP or the production of GPP. Choice of one set of enzymes should direct a cell towards making monoterpenoids or sesquiterpenoids.

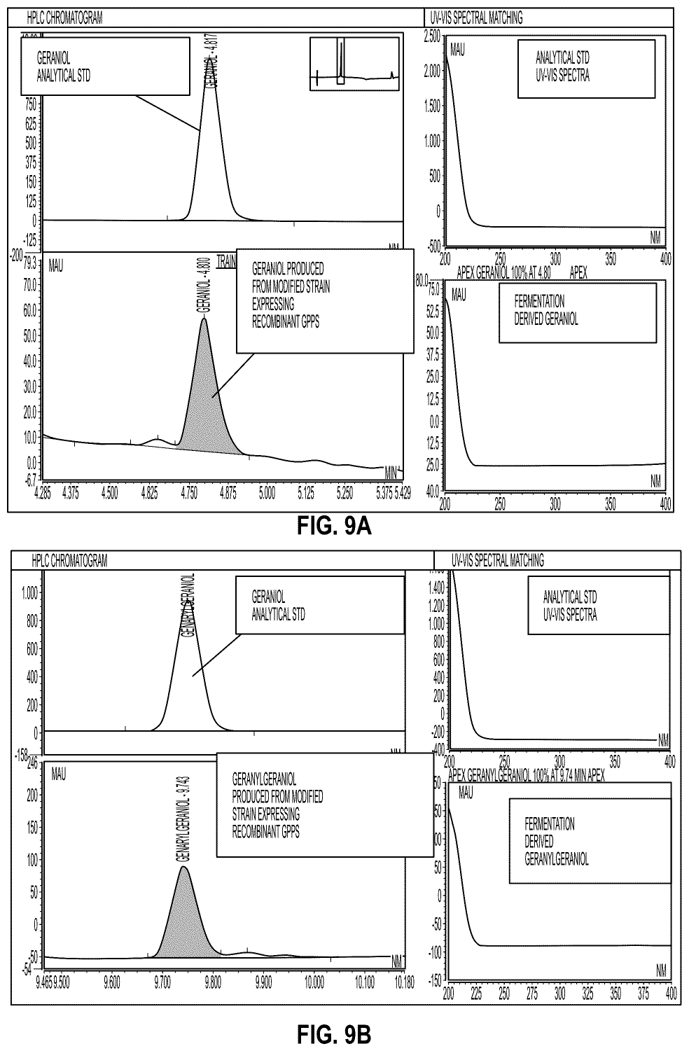

To produce the desired terpene, each candidate polypeptide is introduced into a host cell genetically modified to contain all necessary components for cannabinoid and terpene biosynthesis using standard yeast cell transformation techniques (Green and Sambrook (2012). Cells are subjected to fermentation under conditions that activate the promoter controlling the candidate polypeptide (see, e.g., Table 3). The broth may be subsequently subjected to HPLC analysis (FIG. 9).

DNA sequences encoding the GPPS are synthesized and cloned using techniques known in the art (Green and Sambrook (2012). Gene expression can be controlled by inducible or constitutive promoter systems (see Table 3) using the appropriate expression vectors. Genes are transformed into an organism using standard yeast or fungi transformation methods to generate modified host strains (i.e., the recombinant host organism). To produce cannabinoids, the modified strains which produce cannabinoid precursors express genes for (i) a bacterial GPP synthase, (ii) an archaeal GPP synthase, or (iii) a mixture of archaeal and bacterial GPP synthases to generate meroterpenoids such as CBGA, sesqui-CBGA, CBGGA, and mono-, sesqui- and diterpenes. The modified strains from above can also co-express genes for downstream cannabinoid synthases, such as CBCA, THCA, and CBDA synthases, to produce additional cannabinoid compounds including but not limited to CBCA, CBCVA, CBC, THCA, THCVA, THCV, CBDA, CBDVA, CBD, CBGF, CBGFA, CBDF, CBDFA, THCF, THCFA, etc.

In some embodiments, recombinant heterologous GPPS genes are expressed in combination with a modified cannabinoid producing strain.

Construction of a modified Saccharomyces cerevisiae host is carried out by co-expressing cannabinoid synthases with (i) a rkGPPS enzyme, (ii) a bkGPPS enzyme, (iii) a mixture of either rkGPPS, bkGPPS, or both rkGPPS and bkGPPS enzymes, as shown in FIG. 5. The recombinant GPPS genes expressed with the cannabinoid pathway in a modified host enable the production of cannabinoids, such as CBGVA, CBGA, CBDA, THCA, CBCA, etc. The modified host can also produce sesquicannabinoids, such as CBFA, CBFVA, CBF, THCFA, etc. The optimized GPPS genes are synthesized using DNA synthesis techniques known in the art and expressed in a modified host as referenced, as described in U.S. Provisional Patent Application 63/035,692. Strains with fungal prenyltransferase and mixed prenyltransferase pathways co-expressing downstream cannabinoid synthase genes can be screened by rescue of auxotrophy and genome sequencing.

During cannabinoid biosynthesis a polyprenyl pyrophosphate such as GPP, NPP, FPP, and GGPP acts as a prenyl donor and is combined with a prenyl acceptor to produce a cannabinoid. For example, combining GPP with olivetolic acid (OA) results in the formation of cannabigerolic acid (CBGA) (FIG. 3), which itself is a precursor of other downstream cannabinoids such as cannabidiolic acid (CBDA), cannabichromenic acid (CBCA), tetrahydrocannabinolic acid (THCA). As a direct precursor of CBGA, any increase in the intracellular concentration of GPP should result in increased titers of these cannabinoids. Decarboxylation, which can occur spontaneously or with the addition of heat, leads to cannabinoids such as cannabigerol (CBG), cannabidiol (CBD), cannabichromene (CBC), and tetrahydrocannabinol (THC) (FIG. 3).

When FPP is used in place of GPP during CBG biosynthesis, a prenylog is generated, published as sesquicannabigerol (CBF) (Pollastro, 2011). If the prenylog sesquicannabigerol (CBF) is the desired reaction product, in this case it would be desirable to increase intracellular levels of FPP. This could be accomplished by overexpression of bacterial and archaeal GPP synthase enzymes (GPPSes) that preferentially make FPP.

When GGPP is used in place of GPP during CBGA and CBG biosynthesis, the prenylogs cannabigerogerol (CBGG) and cannabigerogerolic acid (CBGGA) are generated. If the prenylogs CBGG and CB GGA are the desired reaction products, in this case it would be desirable to increase intracellular levels of GGPP. This could be accomplished by overexpression of bacterial and archaeal GPP synthase enzymes (GPPSes) that preferentially make GGPP.

CBGA is a precursor molecule of many downstream cannabinoids, e.g. CBDA, THCA, CBCA. If FPP is used in place of GPP in the biosynthesis of CBGA and the CBGA prenylogs sesquicannabigerol (CBF) or sesquicannabigerolic acid (CBFA) are generated (FIG. 3), sesquicannabigerol or sesquicannabigerolic acid will be the precursor molecule for prenylog versions of the downstream cannabinoids, e.g. sesquiCBDA, (CBDFA), sesquiTHCA, (THCFA), sesquiCBCA (CBCFA), etc.

The alkyl chain of the prenyl acceptor may also vary during cannabinoid biosynthesis. If divarinolic acid, also called divarinic acid or varinolic acid, which has an alkyl chain 2-carbons shorter than olivetolic acid (FIG. 3) is used in place of olivetolic acid and GPP is the prenyl donor, CBGVA will be the product. If sphaerophorolic acid which has an alkyl chain 2-carbons longer than olivetolic acid (FIG. 4) is used in place of olivetolic acid and GPP is the prenyl donor, CB GPA will be the product. The sesqui-versions of CBGVA and CBGPA also exist, formed by using FPP as the prenyl donor and divarinolic acid or sphaerophorolic acid as the prenyl acceptor. Similarly, the diterpenoid variants of CBGVA and CBGPA, formed by using GGPP as the prenyl donor and divarinolic acid or sphaerophorolic acid as the prenyl acceptor.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1. Expression of a Mixed GPPS Pathway for Cannabinoid Production in a Modified Host Organism

Recombinant Saccharomyces cerevisiae were modified to express multiple GPPS genes, following the techniques described in Ignea (2014) and Rubat (2017).

Modification of host cells included expression of genes on self-replicating vectors and/or genetic insertion of recombinant genes by single or double cross-over insertion. Vectors used for modified host cell expression of GPPSes and biosynthetic pathways for terpenes and cannabinoids contained a yeast origin of replication, a promoter upstream of the recombinant gene or fusion-gene, and a poly-A terminator downstream of the recombinant genes or fusion-genes, allowing for expression of recombinant enzymes and fusion-enzymes (Table 1 and 2). In some cases, the vectors contained auxotrophic and drug-resistant markers for host cell selection, such as selectable cassettes for the amino acid, tryptophan, or antibiotic, geneticin. Recombinant genes were cloned into expression vectors using restriction digest and T4 ligation, by techniques known in the art.

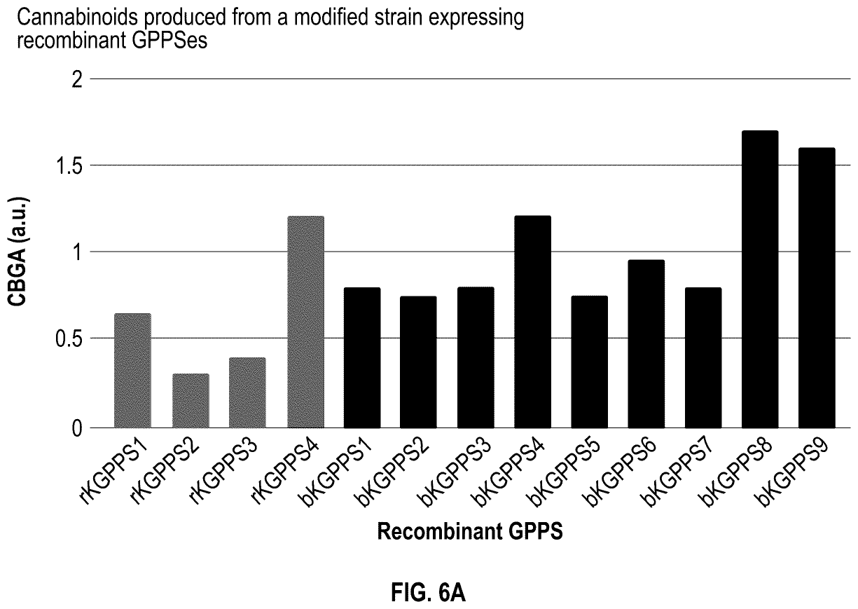

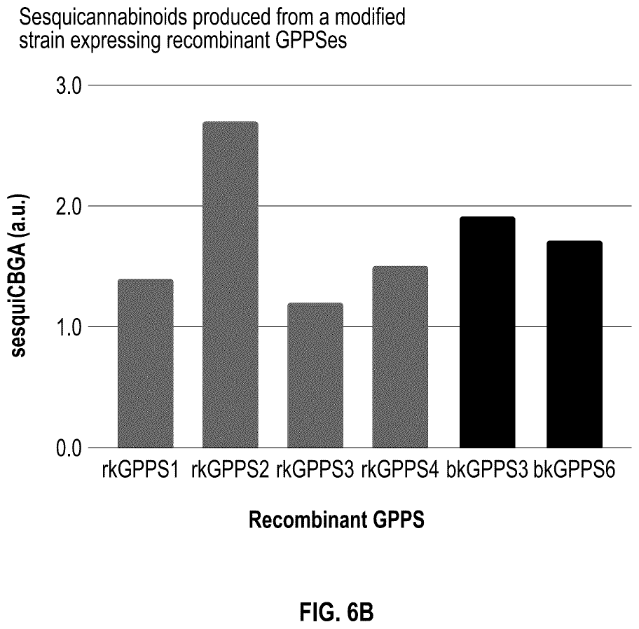

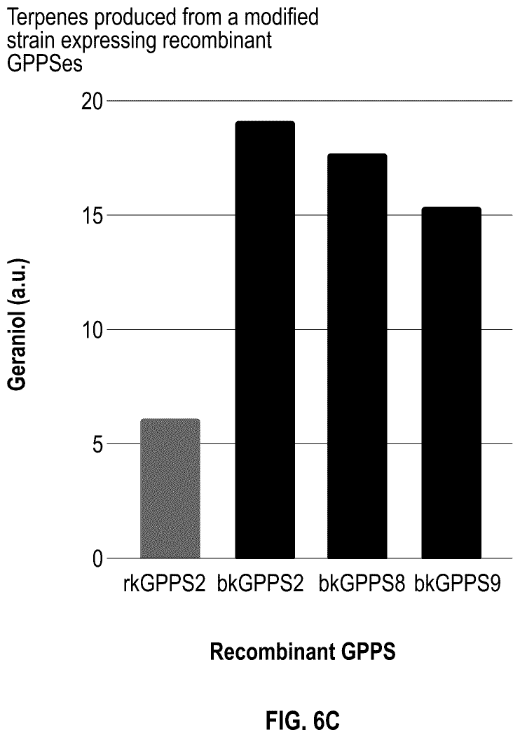

The production of cannabinoids, sesquicannabinoids and terpenes by strains with various recombinant GPPSes is shown in FIGS. 5, 6A, 6B and 6C, using methods described in Example 3. As shown in FIGS. 6A, 6B and 6C, expression of different GPPSs result in differences in absolute amount of cannabinoids, sesquicannabinoids and terpenes produced, as well a different ratios of cannabinoids to sesquicannabinoids and to terpenes.

### Example 2. Methods of Growth

Construction of Saccharomyces cerevisiae strains expressing bacterial or archaeal GPPS enzymes fused with N terminal cofolding peptides from Table 2, SEQ76-SEQ80 to produce GPP, NPP, FPP, and/or GGPP for cannabinoid and/or terpene production, including CBGA or geraniol, was carried out via expression of a fusion GPPS gene of any codon optimized nucleic acid sequence SEQ71-SEQ75 combined at the 5′ end of any nucleic acid sequence SEQ1-SEQ36 which encodes for an enzyme with GPPS activity such as the archaeal (rkGPPS) and bacterial (bkGPPS) genes and proteins listed in Table 1. The fusion GPPS genes were cloned into vectors with the proper regulatory elements for gene expression (e.g. promoter, terminator) and the derived plasmid was confirmed by DNA sequencing. Alternatively, the fusion GPPS genes were inserted into the recombinant host genome. Integration was achieved by a single cross-over insertion event of the plasmid. Strains with the integrated gene were screened by rescue of auxotrophy and genome sequencing.

Cannabinoid-producing strains expressing the GPPSs of the present invention were grown in a feedstock as described in U.S. patent application Ser. No. 17/068,636, in a minimal-complete or rich culture media containing yeast nitrogen base, amino acids, vitamins, ammonium sulfate, and a carbon source, such as glucose or molasses. The feedstock was consumed by the modified host to convert the feedstock into (i) biomass, (ii) GPP, NPP, FPP, cannabinoids and/or terpenes, and (iii) biomass and GPP, NPP, FPP, cannabinoids and/or terpenes. Strains expressing the recombinant GPPS genes were grown on feedstock for 12 to 160 hours at 25-37° C. for isolation of products.

### Example 3. Detection of Isolated Product

To identify fermentation-derived terpenes, cannabinoids, and sesquicannabinoids, (see FIGS. 5, 6A, 6B, 6C, 7A, 7B, 8, 9A and 9B), an Agilent 1100 series liquid chromatography (LC) system equipped with a reverse phase C18 column (Agilent Eclipse Plus C18, Santa Clara, CA, USA) was used with a gradient of mobile phase A (ultraviolet (UV) grade H₂O+0.1% formic acid) and mobile phase B (UV grade acetonitrile+0.1% formic acid), and a column temperature of 30° C. Compound absorbance was measured at 210 nm and 305 nm using a diode array detector (DAD) and spectral analysis from 200 nm to 400 nm wavelengths. A 0.1 milligram (mg)/milliliter (mL) analytical standard was made from certified reference material for each terpene and cannabinoid (Cayman Chemical Company, USA). Each sample was prepared by diluting fermentation biomass from a recombinant host expressing the engineered biosynthesis pathway 1:3 or 1:20 in 100% acetonitrile and filtered in 0.2 um nanofilter vials. The retention time and UV-visible absorption spectrum (i.e., spectral fingerprint) of the samples were compared to the analytical standard retention time and UV-visible spectra (i.e. spectral fingerprint) when identifying the terpene and cannabinoid compounds.

FIGS. 6A, 6B and 6C depict a bar graph of isolated cannabinoid (6A), sesquicannabinoid (6B), and terpene (6C) products from various fermentations of a modified host strain expressing recombinant rkGPPS and bkGPPS genes listed in Table 1.

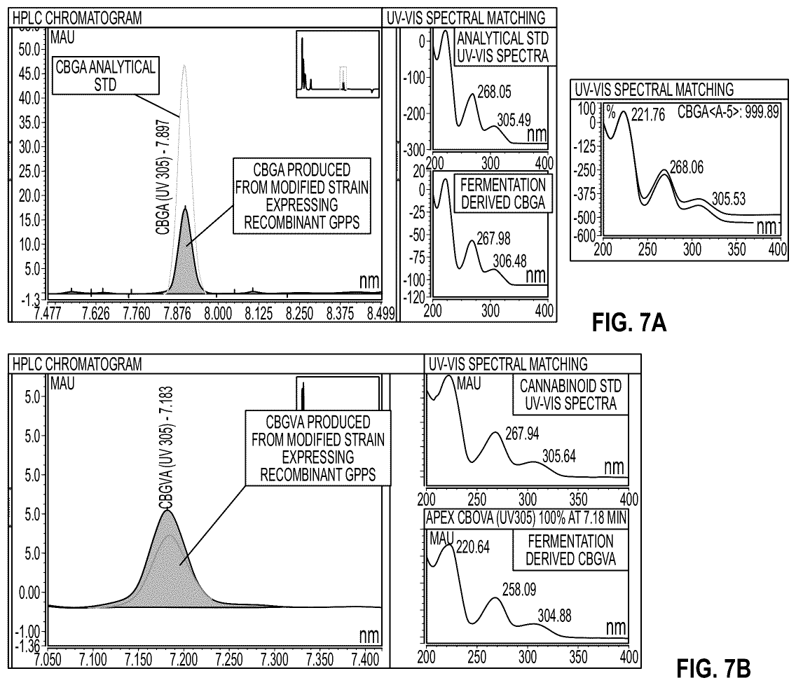

FIGS. 7A and 7B depict the detection of CBGA (7A) and CBGVA (7B) isolated from fermentation with a recombinant host expressing recombinant GPPS enzymes for CBGA and CBGVA production from GPP. Detection and isolation were depicted by retention time matching of fermentation derived CBGA (middle panel) with a CB GA analytical standard (top panel), along with a matching UV-vis spectral fingerprint of the fermentation derived CBGA with the CBGA analytical standard. This also corroborates that the recombinant host is able to successfully convert GPP to CBGA and CBGVA, which further validates that the systems and methods herein direct molecules into cannabinoid pathways from the recombinant GPPS enzymes.

FIG. 8 depicts the identification of CBGA and CBFA, by HPLC chromatogram and UV-vis spectra as described above. The UV-vis spectrum identified the cannabinoid compounds in addition to the retention time matching on the chromatogram.

FIGS. 9A and 9B depicts the HPLC chromatograms and UV-vis spectral matching of the monoterpene geraniol (9A) and the diterpene geranylgeraniol (9B) produced from the fermentation of a modified host strain expressing recombinant heterologous GPPSes. Production of the terpenes were confirmed by comparison with analytical standards by retention time and UV-vis special fingerprinting between the fermentation derived product and the analytical standard.

### REFERENCES

Davis and Croteau R. (2000) Cyclization Enzymes in the Biosynthesis of Monoterpenes, Sesquiterpenes, and Diterpenes. In: Leeper F. J., Vederas J. C. (eds) Biosynthesis. Topics in Current Chemistry, vol 209. Springer, Berlin, Heidelberg. https://doi.org/10.1007/3-540-48146-X_2
Fischer et al. (2011). Biotechnology and Bioengineering 108:1883-1892.
Green and Sambrook (2012) Molecular Cloning: A Laboratory Manual (Fourth Edition), Cold Spring Harbor Laboratory Press, Cold Spring Harbor, N.Y.
Kshatriya (2020), Thujone Biosynthesis in Western Redcedar (Thuja plicata). University of British Columbia Thesis.
Ignea et al. (2014) ACS Synth. Biol. 3:298-306.
Pelot et al. (2016) Plant Journal: Cell and Molecular Biology. 89. 10.1111/tpj.13427.
Pollastro et al. (2011) Nat Prod. 74:2019-22.
Rubat et al. (2017) FEMS Yeast Research 17, 2017 doi: 10.1093/femsyr/fox032.
Vinokur et al. (2014) Biochemistry 53:4161-4168.
U.S. patent application Ser. No. 16/553,103.
U.S. patent application Ser. No. 16/553,120.
U.S. patent application Ser. No. 16/558,973.
U.S. patent application Ser. No. 17/068,636.
U.S. Provisional Patent Application 63/053,539.
U.S. Provisional Patent Application 63/035,692.
US Patent Publication 2020/0063170.
US Patent Publication 2020/0063171.
U.S. Pat. No. 10,435,727.

Sequences

Seq. ID NO: 1
>bkGPPS1
ATGTCATCCGATTCTAGCTCTATAGGGGCGATCGAAACCAGAATACGTGAACTGGTCCATGACTATGT
GGGTGTCAATGGCACTGATGCACCTATAACGCCAGCTTTACGTCCCATGTTTCATACCGTCGTTGACCA
GGCGCTTGCTTCGAGCGAGGGAGGGAAAAGATTACGCGCTCTTTTAACTTTGGACGCATATGATGTCT
TGGCAGGGGCGCCGGATTCTACTCAAAGTAGGTCCGTCAGAACTAAGGTCCTAGATTTCGCGTGCGCT
ATCGAGGTCTTCCAAACCGCGGCGTTGGTACACGATGACCTGATTGATGATAGCGACTTGAGGAGGGG
CAAACCTTCTGCACATTGCGCACTAACATCATTTGCAGGAGCAAGGAGCATAGGTCGTGGACTGGGCC
TTATGCTTGGAGATATGTTGGCTACGGCATGTACGCTGATAATGGAAGACGCTAGTACTGGTATGGTC
GAGCACCGTAGGCTGGTCGAAGCGTTTCTAAGTATGCAGCACGACGTCGAAGTTGGACAAGTGTTGGA
TTTAGCTATCGAAAGAATGCCCCTGGACGACCCACAGGCGCTTGCAGAAGCCAGCCTTGACGTCTTTC
GTTGGAAAACTGCGTCCTACACGACCATAGCACCACTAATGTTGGCTTTCTTAGCAAGTGGTATGACA
AGCGAAGCCGCGAACCTTCACTGTCATGCTATTGGATTGCCGTTAGGCCAAGCATTCCAGCTTGCAGA
CGATCTGTTGGACGTTACAGGAAGTTCTCGTTCTACCGGGAAACCCGTGGGTGGTGATATTAGAGAAG
GTAAAAGAACAGTATTACTTGCAGACGCGATGATGCTAGGGACCGCTGCACAGCGTGTCCAACTACAG
CAATTATATGAGCAACCCTTCAGATCAGATGCGCAGGTTCATGAGACCATTGCTCTATTCCATGATACC
GGCGCGATTGAACACTCACATGAGAGAATAGCTAAGTTGTGGAGTCAAACCCAAGAGTCTATTGAGG
CTATGGGCCTTACAGCCGCTCAGAGTCAGAGCCTGCGTAAGGCGTGCGAGCGTTTCCTACCGGATTTT
ACCGCCGAAAGGTAA
Seq. ID NO: 2
>bkGPPS2
ATGTCATGTACCACTGCTAATAATCGTGAGATCATCGAACCCAGGATCATACAATTAGTCAGGGAACT
TACCGCGGCACCGGCGACCGACGAAGTTGCCGACGCGTTGAAGCCGGTAATGGAACAAGTCGTAGAC
CAGGCCGCCAGTTCTTCCCAAGGCGGGAAGAGACTAAGGGCCCTTTTAGCATTAGACGCCTTCGATAT
TCTTGCAGGTGACGTAACGCCAGATAGGCGTGATGCAATGATTGATCTAGCATGTGCAATCGAAGTGT
TCCAAACTGCGGCGCTGGTTCACGATGACATTATAGACGAAAGCGACCTACGTCGTGGCAAACCCTCA
GCACACCATGCTCTTGAGCAAGCAGTCCATAGCGGCGCGATAGGCAGAGGTTTGGGTCTGATGTTGGG
AGACATCCTTGCAACCGCATGCATAGAAATTACTCGTAGAAGCGCCTCACGTCTTCCTAACACTGACG
CCTTGAATGAGGCGTTCCTAACAATGCAGAGAGAAGTAGAAATTGGTCAGGTACTAGACTTAGCCGTG
GAGATGACTCCTCTGTCTAATCCGGAAGCACTAGCTAACGCAAGCCTAAATGTGTTTAGGTGGAAGAC
CGCTTCATATACGACGATAGCACCTCTATTATTAGCATTACTTGCTGCCGGTGAATCTCCAGATCAAGC
TAGGCACTGCGCCTTAGCGGTCGGGAGGCCTCTGGGGTTGGCCTTTCAATTAGCGGACGATCTGCTAG
ACGTAGTAGGGTCTAGCAGAAATACCGGCAAACCAGTAGGGGGTGACATTAGGGAAGGTAAGAGAAC
AGTGTTGTTGGCCGACGCCTTGTCAGCGGCTGACACGGCTGACAAAGCGGATCTTATAGCGATTTTCG
AGGAGGACTGTAGGAACGATAACCAGGTGGCGAGAACGATCGAATTATTTACATCAACAGGTGCTCT
GGATCGTAGTCGTGAGCGTATAGCTGCATTGTGGGGTGAATCAAGGAAAGCAATCGCTGGATTGGAGT
TGAACTCCGAGGCTCAAAGGAGGCTGACCGAGGCTTGTGCCCGTTTTGTACCGGAAAGTCTTAGATAA
Seq. ID NO: 3
>bkGPPS3
ATGTCAGATAAGATTAAAAAGATGGGCGAGGAAATAGAACTTTGGTTAAAAGAATATTTGGATAATA
AGGGTAACTACGATAAGAAGATATATGAAGCAATGGCTTACTCTTTGGAGGCTGGCGGGAAGAGAAT
TAGACCGGTGCTGTTTCTAAACACTTACTCACTATATAAGGAGGATTACAAGAAAGCAATGCCGATTG
CAGCCGCCATTGAAATGATTCATACATACTTCTTGATACACGATGATCTGCCGGCCATGGACAACGAC
GACTTACGAAGGGGAAAACCCACTAACCATAAAATATTTGGAGAAGCAATAGCGATACTTGCGGGAG
ACGCTCTATTAAATGAAGCAATGAACATAATGTTTGAGTACAGCCTGAAGAATGGGGAAAAAGCGTT
AAAAGCATGTTACACCATTGCTAAAGCTGCGGGAGTCGATGGGATGATCGGAGGGCAAGTCGTAGAC
ATTTTATCAGAAGATAAATCTATCTCATTGGATGAGTTGTATTATATGCACAAAAAGAAAACCGGTGC
CTTAATAAAAGCGTCAATACTTGCTGGAGCCATATTGGGCTCAGCTACCTATACTGATATAGAACTACT
AGGCGAGTACGGGGACAACCTTGGCTTAGCGTTCCAGATCAAAGATGACATACTTGACGTAGAAGGC
GATACAACTACCCTTGGCAAAAAGACGAAAAGCGATGAAGATAATCACAAGACAACCTTTGTTAAAG
TGTATGGAATAGAGAAATGTAACGAACTGTGTACTGAGATGACCAATAAGTGTTTTGACATTCTAAAT
AAGATCAAAAAGAATACTGATAAGTTGAAAGAGATAACGATGTTTCTTCTGAATAGAAACTATTAA
Seq. ID NO: 4
>bkGPPS4
ATGTCAAAAAAGAGGAAGACCCTGGAGGACACAGCAATGAATATCAACAGCCTTAAAGAGGAGGTGG
ACCAATCATTGAAGGCATACTTCAATAAGGATCGTGAGTATAACAAGGTTTTATATGATAGCATGGCT
TACTCAATTAACGTCGGGGGTAAGAGAATAAGACCCATTCTAATGCTGTTGTCATATTACATCTATAA
GTCTGATTATAAGAAAATCCTTACACCAGCGATGGCAATCGAAATGATCCACACTTACTTCATTCACG
ACGACCTACCCTGTATGGACAACGATGATCTAAGGAGAGGAAAGCCGACGAACCATAAAGTGTTCGG
CGAAGCGATAGCAGTATTAGCAGGGGATGCCTTACTAAACGAGGCGATGAAGATACTAGTGGATTAC
TCATTGGAAGAAGGTAAAAGCGCCCTGAAGGCTACGAAAATCATCGCCGATGCAGCGGGATCTGATG
GGATGATCGGAGGGCAAATCGTGGACATCATAAATGAAGATAAGGAGGAAATTTCTCTGAAGGAACT
AGACTATATGCACCTGAAGAAAACTGGCGAGTTAATTAAGGCTAGTATAATGAGTGGTGCAGTCTTAG
CTGAAGCAAGTGAGGGTGACATTAAAAAGCTGGAAGGTTTTGGTTATAAGCTGGGACTGGCTTTTCAA
ATTAAAGATGACATCTTAGATGTAGTGGGTAACGCGAAGGACTTGGGTAAAAATGTCCATAAGGACC
AGGAATCCAATAAAAACAATTACATAACTATCTTTGGTCTTGAAGAGTGCAAGAAAAAGTGCGTTAAT
ATTACAGAGGAGTGCATAGAAATCCTGTCCTCCATAAAAGGGAATACGGAACCCCTGAAGGTCTTGAC
AATGAAACTACTAGAAAGGAAATTCTAA
Seq. ID NO: 5
>bkGPPS5
ATGTCAGACTTTCCTCAGCAATTGGAGGCCTGCGTGAAACAGGCAAATCAGGCGTTGTCCAGATTCAT
TGCACCCTTGCCGTTCCAGAATACGCCTGTAGTTGAGACGATGCAATACGGTGCCCTACTTGGTGGCA
AGAGGCTTCGTCCGTTTCTAGTGTACGCAACTGGACATATGTTTGGGGTATCCACCAACACATTGGAC
GCGCCTGCGGCTGCTGTTGAGTGCATCCATGCCTACTTTTTAATCCACGACGACCTACCCGCCATGGAT
GATGACGATTTAAGACGTGGTTTACCTACGTGCCACGTCAAATTCGGAGAGGCTAACGCAATTCTAGC
CGGGGATGCCCTTCAGACTCTGGCATTTTCCATTCTATCCGACGCCGACATGCCCGAGGTCAGCGACC
GTGACAGGATTTCAATGATCTCTGAATTGGCCTCAGCCAGCGGCATAGCAGGTATGTGTGGAGGTCAA
GCCTTAGACTTGGATGCGGAGGGAAAACACGTTCCCTTGGACGCCCTGGAACGTATTCATCGTCACAA
AACTGGGGCTCTAATTCGTGCTGCCGTCAGGTTGGGTGCGCTTAGTGCAGGTGACAAGGGCAGGAGAG
CTTTACCTGTATTGGATAAGTATGCGGAAAGTATCGGATTAGCTTTCCAAGTCCAAGATGACATTCTGG
ACGTGGTCGGCGATACTGCGACTTTAGGGAAGAGGCAGGGTGCAGACCAGCAGTTGGGGAAGTCAAC
GTATCCTGCTCTATTGGGACTAGAACAAGCTAGGAAAAAGGCCAGGGATTTGATTGATGATGCTAGGC
AGTCACTAAAACAGTTGGCAGAGCAATCACTTGATACTTCAGCTCTTGAGGCCCTGGCCGATTACATT
ATACAGAGAAATAAGTAA
Seq. ID NO: 6
>bkGPPS6
ATGTCAACCAATTTTAGCCAGCAACATCTTCCACTGGTAGAAAAGGTGATGGTTGATTTCATTGCAGA
GTACACTGAGAACGAGAGATTGAAGGAAGCTATGTTGTATTCCATTCACGCTGGAGGGAAAAGGCTG
CGTCCACTGCTGGTCTTAACTACTGTGGCCGCCTTTCAGAAAGAGATGGAAACTCAAGATTATCAGGT
AGCTGCATCCTTGGAAATGATCCATACTTATTTCCTAATACACGACGACCTGCCCGCGATGGATGATG
ATGATTTGAGACGTGGGAAGCCGACAAACCACAAGGTGTTTGGGGAAGCCACTGCTATATTAGCGGG
AGACGGATTATTAACAGGAGCCTTTCAGTTACTATCCTTGAGCCAATTGGGGCTATCCGAAAAGGTAC
TTCTGATGCAGCAGCTGGCGAAAGCTGCTGGTAATCAGGGCATGGTATCCGGACAGATGGGTGATATA
GAGGGGGAAAAAGTGTCTCTGACGCTGGAAGAGCTTGCAGCGGTACACGAGAAAAAGACTGGAGCAC
TGATAGAGTTTGCATTGATTGCAGGAGGCGTCCTAGCAAACCAAACCGAGGAGGTTATTGGTCTGCTT
ACGCAATTCGCGCATCACTATGGATTGGCGTTCCAGATCAGGGACGACCTGCTTGATGCGACTTCAAC
GGAAGCCGACTTGGGCAAGAAAGTTGGTCGTGACGAGGCTCTAAATAAGTCCACATATCCAGCCCTTT
TGGGAATTGCAGGTGCAAAAGACGCTCTAACCCATCAATTAGCGGAGGGCTCCGCTGTGCTAGAGAA
AATTAAGGCAAACGTTCCAAATTTCTCTGAAGAGCACTTGGCTAATCTTCTTACCCAACTGCAATTGAG
GTAA
Seq. ID NO: 7
>bkGPPS7
ATGTCATCTTCCCCTAATCTGTCTTTCTACTACAATGAATGTGAAAGATTTGAATCTTTCCTTAAAAATC
ACCATTTGCACCTAGAAAGTTTTCATCCATACTTAGAGAAAGCATTCTTTGAGATGGTACTGAATGGA
GGAAAGAGGTTCAGGCCTAAGCTATTCTTGGCCGTATTATGTGCGCTAGTCGGTCAGAAGGATTATAG
CAACCAGCAGACGGAGTATTTTAAGATAGCATTGAGCATTGAGTGTTTGCATACATACTTTTTAATCCA
CGATGATTTACCATGTATGGATAATGCTGCTTTGCGTAGGAACCACCCGACTCTACATGCTAAATATGA
TGAGACCACTGCTGTACTAATAGGGGACGCCCTAAACACCTACTCATTTGAACTGTTGAGCAACGCTC
TGCTTGAATCCCATATAATCGTAGAGCTAATTAAGATACTATCTGCAAACGGGGGCATAAAAGGAATG
ATTCTGGGACAGGCATTAGATTGTTATTTCGAGAACACCCCCTTGAACTTGGAGCAGCTGACTTTCCTT
CACGAGCACAAGACTGCTAAATTAATAAGTGCAAGCCTAATTATGGGACTAGTCGCAAGTGGAATTAA
AGACGAGGAGTTGTTCAAATGGCTACAAGCGTTTGGATTGAAGATGGGTCTTTGTTTTCAGGTGTTGG
ACGATATCATAGATGTCACACAGGACGAAGAGGAGTCAGGTAAAACTACACACTTGGATTCAGCTAA
AAACTCCTTCGTGAATCTTCTAGGTTTGGAAAGGGCGAATAATTATGCGCAAACTCTAAAGACGGAGG
TCTTAAACGACCTAGACGCACTGAAGCCCGCCTATCCACTGCTACAGGAAAACCTAAATGCGCTACTT
AATACGCTGTTTAAGGGTAAAACGTAA
Seq. ID NO: 8
>bkGPPS8
ATGTCACCTATAAACGCGAGGTTAATTGCATTCGAGGATCAGTGGGTTCCTGCATTAAACGCTCCGCTT
AAACAAGCGATTCTTGCAGATTCCCACGACGCACAACTTGCTGCCGCTATGACATATTCTGTCCTAGCA
GGGGGAAAACGTTTAAGGCCCCTATTAACTGTCGCAACTATGAGGAGCCTTGGTGTGACTTTTGTACC
TGAGAGACACTGGAGACCCGTAATGGCACTAGAGTTGCTGCATACCTACTTTTTGATTCATGATGATCT
TCCCGCTATGGATAACGACGCATTAAGGAGAGGGGAACCCACCAATCATGTGAAGTTCGGTGCCGGTA
TGGCCACATTGGCAGGGGATGGGCTTTTAACACTAGCGTTTCAGTGGTTGACCGCTACTGACTTGCCA
GCGACTATGCAAGCCGCTCTAGTACAAGCTCTAGCAACCGCGGCAGGCCCTTCAGGCATGGTAGCTGG
TCAGGCGAAAGACATACAGAGCGAACACGTGAATCTACCATTAAGCCAACTTAGAGTATTACATAAA
GAGAAAACAGGCGCTCTACTGCATTACGCCGTGCAGGCAGGATTGATATTGGGCCAAGCCCCAGAGG
CACAATGGCCAGCCTACCTGCAATTTGCGGACGCATTCGGTCTAGCGTTCCAAATATATGATGACATA
TTAGATGTAGTTTCATCTCCGGCGGAGATGGGAAAGGCTACACAGAAGGATGCTGATGAGGCTAAAA
ACACATATCCGGGTAAGCTGGGTCTAATTGGAGCCAATCAAGCTCTAATAGATACTATCCATTCTGGA
CAAGCAGCACTGCAAGGATTACCAACATCCACACAAAGAGATGATCTGGCTGCTTTCTTCTCATACTTT
GATACGGAGAGGGTCAACTAA
Seq. ID NO: 9
>bkGPPS9
ATGTCAGATACCAAGATTTTGAAACTTGAGGACTTCCTAACAGAATTTTATGAGAGTGCAGAGTTCCC
GACTGGGCTGGCCGAATCAGCAAAATACAGTCTACTTGCAGGAGGGAAAAGAATACGTCCGCTATTAT
TTTTGAACCTGCTAGAAGCCTTCGACTTGGAACTTTCTAAGGCTCACTACCATGTCGCAGCAGCTTTGG
AGATGATACATACCGGATCTCTTATCCATGACGATCTTCCAGCAATGGATAATGACGACTATAGACGT
GGCCAATTGACGAATCACAAAAAGTTCGATGAGGCGACAGCTATCTTAGCTGGCGATACCTTATTTTT
CGATCCCTTCTTTATTCTGTCCACTGCGGATTTGAGTGCAGAGATAATCGTTGCCCTAACGAGAGAGTT
GGCTTTCGCCTCTGGCTCATACGGCATGGTCGCGGGGCAAATCTTAGATATGGCAGGTGAAGGAAAAG
AACTAACCCTTGCTGAAATTGAGCAAATCCACAGGCTAAAGACCGGGCGTCTGTTGACGTTCCCTTTC
GTGGCAGCGGGGATTGTCGCCCAAAAGAGTACGGATGAAGTCGAAAAACTAAGGCAAGTGGGGCAAA
TCTTAGGACTTGCTTTCCAAATCAGGGACGACATCCTGGATGTTACAGCGACCTTCGCCGAGCTTGGCA
AAACCCCCGGCAAGGACATTTTAGAGGAGAAGAGTACATATGTAGCTCATTTGGGCTTGGAAGGAGCT
AAAAAGTCTTTGACGGGGAACTTGTCAGAGGTGAAGAAACTACTTACAGATTTATCAGTCACTGATAG
TAGCGAGATTTTTAAGATAATTGAGCAACTGGAAGTTAAGTAA
Seq. ID NO: 10
>bkGPPS10
ATGTCAATAGATTTAAAATCTTTCCAAAAAGAGTGGCTACCAAAAATAAACCAACAACTTGAAAACGA
CCTTAGCATGGCAAGCCCAGACGCGGATCTAGTTGCAATGATGAAATACGCTGTCTTAAATGGTGGAA
AGCGTTTGCGTCCTTTACTTACTCTTGCTGTAGTTACCTCATTCGGGGAATCCATTACACCATCCATTCT
GAAGGTAGCAACAGCGATTGAGTGGGTACATAGCTACTTTCTGGTACACGATGATCTTCCAGCCATGG
ATAACGATATGTTTCGTAGAGGCAAACCTTCCGTCCATGCGCTTTATGGTGAAGCTAACGCAATTTTAG
TAGGCGATGCGTTATTAACGGGCGCTTTTGGCGTCATAGCTACCGCTAATAGTTCTTGTTCCGTCGAAG
ACTGCCTGCCCACAGAAGAGCTGCTTTTGATAACCCAGAACCTGGCGAGAGAAGCCGGAGGTTCAGG
CATGGTCTTAGGACAATTGCATGACATGGATAACCACACTGAAGAGCAGAATGCTTCTACGAATTGGC
TATTGAACGATGTGTACTCAATGAAGACGGCAGCTCTTATACGTTATACGACGACACTAGGCGCTATC
TTGACCCACCAGAACGTCAATGTGGAAGATAATCACTTTGACCCCAAAAAGGCAATGTACGACTTTGG
GGAAAAATTCGGATTAGCATTCCAGATACAAGATGATCTTGATGATTACCAGCAGGACCAGCTTGAGG
ACGTAAATTCACTACCCCATATCGTAGGTGTGAAGGAAGCACAGTCTGTGCTAGATCAGTACCTATTC
TCAACTCAAGAGATACTAGCGAACACTGTTGAGCAGGATCAGCAATTCGACAGGAGGCTGTTAGATG
ACTTTGTATCTCTAATAGGAGACAAGAAGTAA
Seq. ID NO: 11
>bkGPPS11
ATGTCACAGGATTTGACTCTATTCTTGGAACAATATAAAAAGGTCATCGACGAAAGCCTGTTTAAAGA
GATATCAGAGCGTAACATCGAGCCGAGATTAAAAGAGTCTATGTTATACTCTGTCCAAGCGGGCGGTA
AGCGTATAAGGCCCATGTTGGTCTTTGCCACCCTTCAAGCTCTAAAAGTCAACCCTTTACTGGGGGTTA
AAACTGCGACAGCCCTGGAGATGATTCATTTCACCTACTTTCTAATTCACGACGACCTGCCCGCTATGG
ACAATGATGACTACAGGAGGGGTAAATACACGAACCATAAGGTATTTGGAGACGCCACTGCAATCCT
AGCGGGAGACGCCCTTCTAACGTTGGCATTTAGTATTCTGGCCGAAGACGAGAACTTGTCATTTGAGA
CCAGAATAGCATTAATAAACCAAATCTCTTTCAGCTCTGGAGCTGAGGGGATGGTCGGAGGACAACTA
GCAGACATGGAAGCAGAAAATAAACAAGTCACTCTTGAGGAATTATCTTCAATTCATGCAAGGAAGA
CTGGAGAGCTACTGATTTTTGCGGTAACCTCAGCCGCTAAGATAGCAGAGGCGGACCCGGAACAGACT
AAGAGACTAAGGATATTTGCTGAGAATATTGGGATAGGATTTCAGATTTCTGATGACATACTAGATGT
TATTGGCGACGAGACAAAAATGGGGAAAAAGACAGGAGTCGATGCCTTCCTGAATAAGTCTACCTAT
CCTGGTTTGTTGACCTTAGACGGCGCGAAGAGAGCTTTAAACGAGCATGTGGCAATAGCTAAATCCGC
TCTGTCAGGGCATGATTTCGATGACGAAATACTTTTAAAACTGGCAGACCTAATTGCCCTTCGTGAAA
ATTAA
Seq. ID NO: 12
>bkGPPS12
ATGTCAACCGGTGCTATTACGGAACAACTAAGACGTTACTTACACGATAGAAGGGCAGAAACAGCGT
ACATAGGTGACGATTACTCAGGGCTGATAGCAGCCTTAGAGGAGTTCGTGCTAAACGGGGGAAAGAG
ACTGAGGCCCGCCTTCGCGTATTGGGGTTGGCGTGCTGTTGCGACCGAGGCTCCAGATGACCAGGCAT
TATTGTTGTTTTCAGCCCTGGAGCTTCTACACGCATGTGCTCTTGTTCACGATGACGTTATTGACGACA
GTGCGACGAGACGTGGACGTCCGACAACCCACGTCAGGTTTGCTAGTCTACATAGGGATAGACAATGG
CAGGGCTCTCCGGAAAGATTCGGAATGAGTGCAGCAATATTATTAGGTGATCTGGCCCTAGCGTGGGC
GGATGACATCGTATTAGGGGTGGACCTAACACCACAAGCCGCCAGGAGGGTAAGGAGAGTATGGGCT
AACATAAGGACAGAAGTCTTAGGCGGGCAGTATCTGGACATTGTCGCCGAGGCATCAGCTGCTGCTTC
AATCGCCTCCGCCATGAACGTGGACACTTTTAAAACGGCATGTTACACGGTCTCTCGTCCTTTACAACT
TGGGGCAGCTGCGGCGGCCGATAGGCCAGACGTTCATGACCTTTTCTCTCAGTTCGGAACTGACCTGG
GTGTTGCCTTCCAGCTTCGTGATGACGTTCTGGGGGTATTTGGTGATCCAGCGGTAACCGGTAAACCAA
GTGGTGATGACTTGAGATCCGGGAAAAGAACGGTTTTGTTAGCAGAAGCCGTAGAGCTGGCTGAGAA
GTCTGATCCACTAGCGGCCAAATTACTTCGTGACAGCATAGGCGCTCAGTTGTCAGATGCGGAGGTAG
ATCGTCTTCGTGACGTTATCGAATCAGTTGGTGCATTGGCTGCTGCCGAGCAAAGGATCGCTACTTTGA
CACAGAGGGCACTGGCCACCCTGGCGGCTGCACCTATTAACACTGCGGCAAAAGCAGGCCTGAGTGA
ACTAGCGAAACTAGCCACGAATCGTTCCGCTTAA
Seq. ID NO: 13
>bkGPPS13
ATGTCAATCCCTGCCGTAAGTCTGGGCGATCCCCAATTTACAGCAAACGTGCATGATGGCATTGCTAG
GATCACCGAACTGATTAACAGTGAACTTTCTCAAGCTGACGAGGTAATGAGAGACACAGTTGCACATT
TGGTAGACGCTGGTGGTACTCCATTTAGACCTCTATTCACCGTTCTTGCCGCGCAGTTGGGTAGCGATC
CAGATGGGTGGGAAGTTACGGTGGCGGGTGCAGCCATCGAACTGATGCACCTGGGAACTTTGTGCCAT
GATCGTGTGGTAGATGAATCTGATATGTCTAGGAAAACGCCTAGTGACAATACTAGGTGGACCAATAA
CTTTGCAATATTAGCTGGTGACTACAGATTCGCTACCGCAAGTCAGCTTGCAAGTCGTCTTGATCCTGA
GGCTTTTGCGGTCGTCGCGGAGGCGTTCGCGGAGCTTATTACCGGTCAGATGCGTGCAACACGTGGCC
CCGCAAGCCACATAGACACGATCGAACATTACCTTAGGGTGGTCCACGAAAAGACAGGCTCTCTGATT
GCGGCATCTGGACAGCTTGGTGCTGCTTTATCCGGCGCAGCAGAGGAACAGATTAGAAGGGTAGCTCG
TTTAGGAAGGATGATAGGAGCTGCTTTCGAGATTTCAAGAGATATCATTGCTATTTCAGGCGATTCTGC
TACGTTATCAGGCGCGGACCTGGGACAGGCCGTCCACACGTTGCCAATGCTGTACGCACTGCGTGAAC
AAACCCCGGACACGTCTAGGTTAAGGGAGCTATTAGCGGGTCCTATCCATGATGACCATGTCGCAGAG
GCCCTTACTCTGCTAAGGTGCAGTCCGGGTATAGGGAAGGCCAAGAACGTGGTGGCCGCTTACGCTGC
CCAAGCTAGAGAAGAGCTGCCATATCTGCCAGACAGACAACCGAGACGTGCGTTGGCTACCTTGATTG
ATCACGCTATATCCGCCTGTGACTAA
Seq. ID NO: 14
>bkGPPS14
ATGTCAAAATTCAAGGATTTCAGCAATAGGTATCTTCCCGAAATCAACAACGACCTGAGCAACTATTT
CGCGGACAGGGATGACGACATCTTCCGTATGATAACATACGCTTTAAATTCAACGGGAAAGAGACTAA
GACCGCTACTGACATTGGCAACTTTCGCGGCGGCGGGAAATGTTATCAACGATTCCACCATTGAAGCT
GCGACTGCCGTAGAATTTGTTCATGCCTACTTTCTGGTGCACGACGATCTGCCCGAGATGGATGACGA
CACCAAAAGAAGGAACCAATCTTCCACTTGGAAGAAGTTCGGCGTAGGGAACGCCGTATTGGTGGGG
GATGGTTTGCTGACCGAGGCGTTCAAAAAGATTTCTAACTTATCTTTGCCTGAGTCCATAAGGTTAAGA
TTGATTTACAATCTTGCTCTTGCCGCCGGTCCGGATAACATGGTGCGTGGACAGCAATACGACCTATTC
AGTCAAGACAAGGTCGAGTCCATAGATGACCTGGAGTTCATCCATTTGATGAAAACTGGCGCTTTGAT
GACTTACGCAGCTACTGCAGGTGGGATACTAGCCGGGCTGAGCGATGATAAGCTGAGGGCATTGAAC
ATATATGGGGCTAATCTGGGAATAGCGTTTCAGATTAAGGACGATCTAAGGGACATAAAACAGGATG
AAGAGGAAAATAAAAAGTCATTCCCCCGTTTAATTGGTGTTCAAAAATCCCAGACAGAGCTAGAAGA
ACACTTAAAGATTTCAGCCAACGCGATCAAAGAAATCCCGGACTTTCAGAATACAGTCCTGCTGGACC
TACTTGACAGAATTTAA
Seq. ID NO: 15
>bkGPPS15
ATGTCAGAAGCCGTCCTGTCCGCCGGTGCAGGCGAATCAACGAGACCATCTCCCAGTGTTCCTCCTTTT
ACGGATACTGTTGAAGACGCTCTTCGTGAATTTTTCGCGAGTAGAGCAGGGACGGTCGAAACTGTAGG
TGGCGGTTACGCGGAAGCAGTCGCTGCCCTAGAGAGTTTTGTCCTGAGAGGTGGTAAGAGGGTTAGGC
CGATGTTTGTGTGGACGGGATGGTTGGGGGCTGGTGGAGACGCAACCGGGCCTGAGGCGCCTGCCGCT
TTGCGTGCGGCGTCCGCATTGGAGTTGGTTCAAGCATGCGCCTTAGTTCATGACGACATAATTGACGCT
TCCACTACGAGAAGAGGATTTCCAACTGTCCATGTTGAATTTGCTGACCAGCATTCAGCTCATCATTGG
TCCGGTGGCTCAGCTGAATTTGGTCGTGCAGTGGCTATCCTTTTGGGGGATTTGGCGTTGGCTTGGGCA
GATGACATGATTAGAGAAGCGGGCCTGAGTCCCGATGCTCAGGCGCGTATTTCCCCAGTTTGGTCTGC
AATGAGAACCGAAGTTCTGGGAGGTCAATTCCTTGATATAAGCTCTGAAGTGAGAGGCGACGAAACT
GTCGAGGCAGCATTACGTGTAGACAGGTACAAAACAGCGGCTTATACTATCGAGCGTCCCTTGCATCT
AGGTGCTGCGTTGGCTGGAGCGGATGATGCGTTAGTAGCGGCGTACCGTACCTTTGGCACTGATATAG
GTATCGCGTTCCAGCTACGTGATGACCTGTTGGGTGTCTTTGGAGACCCCGAGATCACAGGGAAGCCC
TCCGGCGATGATTTGAGAGCTGGCAAAAGGACCGTTCTGTTTGCTGAGGCATTGCAACGTGCAGACGC
CAGTGATCCTGCGGCGGCTGCACTTCTAAGGGAATCCATTGGGACAGACTTGAGCGATGCGCAGGTAG
CTACACTTAGGAGCGTCATTACGGACTTAGGGGCTGTCGATGACGCAGAAAGGCGTATCTCTGAACTT
ACCGACAGTGCTTTATCTGCTTTGGACGGGTCTACAGCGACTGACGAAGGTAAGCTGCGTTTGAGGGA
AATGGCCATTGCCGTAACGAGAAGAGACGCCTAA
Seq. ID NO: 16
>bkGPPS16
ATGTCAGACTTCCCACAACAGCTAGAAGCGTGTGTCAAACAAGCTAACCAGGCTTTGTCAAGATTTAT
AGCTCCGCTGCCCTTCCAGAATACTCCGGTAGTGGAGACCATGCAGTACGGGGCATTGTTGGGCGGGA
AGAGGCTACGTCCGTTTCTGGTATACGCAACCGGTCATATGTTTGGGGTCAGCACGAACACACTGGAT
GCTCCCGCCGCAGCTGTTGAGTGTATTCACGCATACTTTTTGATCCACGACGATTTACCGGCAATGGAT
GACGACGACTTGCGTAGAGGACTGCCTACTTGTCATGTTAAATTTGGCGAAGCCAATGCCATACTGGC
GGGGGACGCATTGCAGACCTTGGCGTTTAGCATTCTTTCCGACGCTAATATGCCGGAGGTTTCTGATCG
TGACAGGATCTCCATGATTTCTGAGTTGGCTTCTGCGTCCGGCATTGCAGGAATGTGTGGTGGACAAG
CACTTGATTTAGACGCTGAGGGAAAGCACGTACCGCTGGACGCTCTGGAACGTATCCATCGTCACAAA
ACCGGCGCACTGATACGTGCTGCTGTTAGACTAGGTGCTCTAAGTGCCGGGGACAAGGGAAGGAGAG
CCCTTCCTGTCTTAGACAAATATGCAGAAAGTATAGGACTAGCTTTTCAAGTACAGGACGACATATTA
GATGTGGTCGGCGATACGGCAACTTTGGGGAAACGTCAGGGCGCTGATCAACAGCTGGGTAAATCCA
CGTATCCAGCACTTCTAGGTCTGGAGCAGGCTCGCAAGAAAGCGAGAGATTTAATCGACGACGCACGT
CAGGCACTTAAACAATTAGCGGAGCAAAGCCTGGACACATCCGCGTTAGAGGCTTTGGCTGACTACAT
AATACAGAGGAACAAATAA
Seq. ID NO: 17
>bkGPPS17
ATGTCAAAAGATAAGATTAAGTATATTAACCAAGCCATAAAGCATTACTACGCACAGACGCATGTGTC
TCAGGACTTAGTGGAAGCAGTGCTTTACTCTGTCGCCGCTGGTGGAAAAAGGATACGTCCCCTTTTGCT
GCTTGAAATCCTGCAAGGGTTTGGTCTTGTATTAACCGAAGCCCATTACCAGGTTGCAGCAAGTTTAG
AAATGATACACACTGGTTTTCTAGTCCATGACGACCTTCCCGCTATGGACAACGATGACTACAGACGT
GGCCAGCTAACTAACCACAAGAAATTCGGTGAAACTACGGCCATACTTGCTGGGGATTCCCTTTTCCT
AGACCCCTTCGGCTTACTAGCGAAGGCCGATTTGCGTGCCGACATCAAAATCAAGTTGGTTGCGGAAC
TATCTGACGCAGCTGGAAGCTATGGCATGGTAGGCGGCCAGATGTTGGATATTAAGGGAGAGCATGTG
CAGCTGAATTTAGACCAACTTGCCCAGATACACGCTAACAAGACTGGAAAGCTATTAACCTTCCCATT
TGTGGCAGCCGGCATCATTGCAGAGCTATCCGAAAAAGCACTGGCTAGGCTGCGTCAAGTGGGGGAA
TTAGTTGGCTTGGCCTTTCAGGTCAGGGATGACATCTTAGACGTTACGGCGAGTTTTTCTGAACTTGGC
AAGACCCCTCAGAAAGACATAGAAGCTGATAAGTCTACATATCCCTCATTACTGGGTCTGGATAAATC
CTACGCTATACTGGAGGACAGTCTGAACCAGGCCCAGGCAATTTTCCAAAAGCTGGCCCTAGAGGAAC
AGTTCAACGCAACAGGTATTGAGACGATAATTGAACGTCTACGTCTACACGCGTAA
Seq. ID NO: 18
>bkGPPS18
ATGTCACAAGAGGCGTTAATCAGCTTTCAACAGAGGAACAATCAGCAGTTGGAGTGGTGGCTTTCTCA
GCTACCTCACCAGAACCAGACTTTGATCGAGGCGATGAGATACGGGCTACTATTGGGCGGTAAAAGG
GCAAGGCCCTTTCTGGTATACATCACCGGACAAATGCTGGGCTGTAAGGCCGAAGATTTAGATACGCC
TGCCAGTGCGGTCGAATGTATTCATGCGTATTCTCTGATTCATGACGACTTACCTGCTATGGATGACGA
TGAGTTGAGACGTGGACAACCAACTTGTCATATAAAGTTCGATGAAGCCACAGCAATTTTAACTGGGG
ACGCATTACAAACACTTGCGTTTAGCATATTGGCCGACGGACCGCTAAACCCCAACGCTGAGTCAATG
AGAATCAACATGGTAAAGGTATTAGCTCAGGCTTCAGGTGCCGCAGGTATGTGTATGGGCCAAGCGTT
GGATTTGCAGGCGGAGAACAGGTTGGTGAATCTTCAAGAACTTGAGGAAATACATAGAAACAAGACG
GGGGCTCTGATGAAATGTGCGATACGTCTAGGCGCACTAGCTGCGGGAGAGAAGGGGCGTGAAGTGT
TACCCTTACTAGACAAGTACGCCGACGCGATAGGATTGGCCTTTCAAGTTCAAGATGATATCTTGGAC
ATTATTAGTGACACCGAAACATTGGGGAAGCCGCAGGGTTCTGACCAGGAACTTAATAAGTCCACATA
TCCGGCTCTTCTAGGACTTGAGGGCGCTATTGAAAAAGCAAATAATTTGTTACAAGAGGCCCTTCAAG
CGCTGGATGCAATTCCATACAACACCGAGCTTCTGGAGGAATTTGCCAGATATGTTATCGAGCGTAAA
AACTAA
Seq. ID NO: 19
>bkGPPS19
ATGTCACACAAGCCCGTTGATCTGACGGATACGGCGGCCTTCGAGACCCAGTTAGACAGATGGAGGG
GTAGAATCGGAGAGGCCGTTGCTGAAGCGATGGCATTTGGCACGACGGTGCCAGCACCGTTACAGGCT
GGGATGTCTCACGCCGTCCTGGCTGGGGGAAAGAGGTACCGTGGAATGCTAGTGCTGGCGCTGGGTTC
AGACTTGGGGGTGCCTGAGGAGCAGTTACTAAGCAGCGCTGTCGCGATAGAGACCATCCACGCGGCCT
CATTGGTTGTAGACGACCTGCCTTGCATGGACGACGCCCGTCGTAGGAGGTCCCAACCCGCCACGCAC
GTGGCATTTGGCGAAGCGACAGCTATTTTATCTAGTATCGCGCTGATTGCTCGTGCGATGGAGGTTGTC
GCGAGAGACAGGCAATTAAGTCCTGCGTCCAGATCTTCAATAGTTGACACACTATCTCACGCAATAGG
GCCACAGGCCTTATGTGGCGGGCAATACGACGACTTATATCCGCCCTATTACGCAACGGAACAAGATC
TTATACACCGTTATCAAAGAAAGACCAGCGCATTATTTGTGGCCGCTTTCCGTTGTCCTGCATTATTAG
CTGAGGTAGACCCTGAAACTCTATTAAGGATAGCGCGTGCCGGACAAAGGCTGGGTGTTGCTTTCCAG
ATATTCGACGACCTGTTGGATCTGACTGGAGATGCACACGCCATAGGGAAAGATGTCGGACAGGACC
ACGGCACCGTTACACTGGCAACTTTATTAGGACCAGCTAGAGCGGCGGAAAGGGCTGCCGATGAGCT
AGCTGCCGTACAGAAAGAGCTTCGTGAAACTGTGGGGCCGGGTCGTGCCTTAGACTTGATTAGACGTA
TGGCCGCACGTATAGCTGGGACTGGAAAAAAATCTGCAGGCCGTGATGATCTAAGGCCTCATGCTGGA
Seq. ID NO: 20
>bkGPPS20
ATGTCAGCATTCGAGCAGCGTATTGAGGCGGCTATGGCCGCCGCGATAGCTAGAGGACAGGGGTCAG
AAGCCCCGTCAAAATTGGCCACAGCTCTAGATTACGCCGTCACTCCAGGTGGAGCCCGTATTCGTCCA
ACCTTATTATTAAGCGTTGCGACGAGGTGTGGCGACAGTAGACCTGCGCTTTCCGATGCCGCCGCTGT
GGCTCTAGAATTGATCCACTGCGCTTCATTGGTACATGACGACCTTCCGTGTTTTGATGATGCCGAGAT
AAGGAGAGGGAAGCCGACTGTGCATAGGGCCTACTCAGAGCCTCTGGCTATTCTAACGGGCGACTCTC
TGATAGTTATGGGCTTCGAGGTCTTGGCTGGTGCGGCGGCTGATAGGCCACAGAGGGCGTTACAGTTA
GTAACGGCACTAGCGGTCAGGACGGGAATGCCAATGGGAATATGCGCAGGGCAGGGTTGGGAATCTG
AAAGTCAGATCAACTTAAGCGCTTACCACAGAGCTAAAACTGGTGCCCTTTTCATAGCAGCCACGCAG
ATGGGGGCTATTGCAGCCGGTTATGAAGCGGAACCGTGGGAAGAACTGGGAGCGAGGATTGGAGAGG
CATTCCAGGTCGCAGATGATCTGAGAGATGCTCTGTGTGATGCCGAAACCCTAGGCAAGCCAGCTGGG
CAAGATGAAATACATGCTAGGCCTAGTGCAGTTAGGGAATATGGTGTCGAAGGTGCAGCGAAAGGCC
TGAAAGACATTTTGGGAGGGGCCATAGCGTCTATCCCCAGCTGTCCTGCTGAGGCCATGCTAGCCGAG
ATGGTCCGTAGATATGCCGACAAGATTGTGCCTGCCCAGGTGGCCGCTAGAGTC
Seq. ID NO: 21
>bkGPPS21
ATGTCAGCCCTTACTTTACCTGACGCTCAACCCCCTACAGGATTGCTTCCCCTTGAGCAAGCGTGGCTT
CAGCTGGTCCAGACGGAGGTCGAGACATCTCTGGCCGAGCTATTCGAACTGCCCGATGAAGCGGGCCT
AGACGTGAGGTGGACACAGGCATTAACTCAAGCACGTGCGTACACCCTAAGACCGGCAAAAAGGCTA
CGTCCAGCTTTGGTAATGGCAGGACACTGCCTGGCACGTGGCTCAGCCGTTGTCCCGAGTGGGCTTTG
GAGGTTCGCCGCTGGTTTAGAACTACTACATACATTTTTACTGATTCATGACGACGTAGCAGACCAAG
CAGAGCTGAGAAGGGGGGCTCCACCCCTACATCGTATGTTGGCTCCCGGAAGAGCAGGAGAAGATTT
AGCCGTTGTAGTGGGTGATCACTTATTTGCCAGGGCACTTGAAGTGATGCTTGGATCAGGACTTACTTG
TGTCGCTGGTGTGGTCCAGTATTATCTAGGTGTATCCGGTCACACTGCGGCGGGGCAATACTTAGATCT
TGATCTAGGCAGAGCCCCGTTAGCGGAGGTAACCTTGTTCCAAACATTACGTGTCGCTCACTTAAAAA
CGGCCAGATACGGCTTTTGCGCACCTTTGGTCTGTGCCGCAATGTTAGGAGGCGCATCCAGCGGGCTT
GTAGAAGAGTTAGAACGTGTCGGTAGACATGTTGGGCTGGCTTATCAACTGAGAGATGATTTACTTGG
ACTATTTGGAGATAGCAACGTAGCGGGAAAGGCGGCAGATGGGGACTTTCTTCAGGGTAAACGTACCT
TTCCGGTTTTAGCAGCCTTTGCCCGTGCAACGGAAGCAGAAAGAACAGAACTTGAAGCCCTGTGGGCT
CTTCCGGTAGAGCAGAAGGATGCAGCAGCACTGGCCAGGGCTAGGGCATTGGTCGAGTCTTGCGGAG
GTAGGGCGGCTTGTGAAAGGATGGTTGTAAGGGCGTCCAGGGCGGCCAGGCGTTCCCTGCAAAGTTTA
CCCAATCCTAACGGAGTCAGAGAACTGTTAGATGCCCTGATTGCGAGGCTGGCGCACAGAGCAGCT
Seq. ID NO: 22
>bkGPPS22
ATGTCAGAGGCCACATTGTCTGCAGGGACTGCCAGGGTTGGCCAGTCAAGCACAAACACTGCGCCACA
TCCTACATCTCTTGAACTTCCGGGTGTGTTCGAGGGTGCCCTGCGTGATTTCTTTGATTCTAGAAGGGA
ACTGGTAAGCAATATCGGAGGCGGTTATGAGAAGGCAGTTTCAACACTGGAGGCTTTTGTACTTAGGG
GAGGTAAAAGAGTTAGGCCCAGTTTTGCTTGGACAGGTTGGTTAGGCGCGGGGGGAGACCCTAACGG
GAGTGGCGCGGACGCAGTCATCAGAGCGTGTGCTGCTCTGGAGCTTGTTCAAGCATGTGCCCTAGTCC
ACGATGATATAATCGATGCTTCCACTACTCGTAGAGGCTTTCCTACTGTTCATGTTGAATTTGAAGACC
AGCATCGTGGAGAGGAATGGTCTGGGGACTCCGCGCACTTTGGGGAGGCCGTTGCAATTTTGTTAGGG
GATTTAGCCCTGGCTTGGGCAGATGATATGATTAGAGAAAGCGGGATTTCTCCCGATGCGGCAGCTAG
GGTAAGTCCTGTATGGTCTGCGATGCGTACCGAGGTACTGGGAGGACAATTTCTTGATATTTCCAACG
AAGCCCGTGGCGACGAAACCGTGGAAGCAGCTATGCGTGTTAACAGATACAAAACAGCCGCTTACAC
CATAGAACGTCCGTTACACTTAGGTGCGGCGCTTTTCGGCGCGGACGCTGAGCTAATCGATGCTTATC
GTACATTTGGCACGGACATCGGGATCGCGTTTCAATTAAGGGATGATTTATTGGGAGTTTTTGGTGATC
CTTCTGTCACGGGTAAGCCATCTGGCGACGACTTGATAGCCGGCAAAAGAACAGTTTTGTTTGCAATG
GCCTTAGCTAGAGCTGACGCGGCGGATCCGGCTGCCGCCGAGTTACTTAGAAACGGCATCGGCACACA
GCTAACGGACAATGAAGTGGATACGTTGAGACAGGTAATAACTGACCTGGGTGCGGTAACGGATGTC
GAGACTCAGATTGATACGTTAGTCGAGGCGGCAGCCAACGCACTTGACAGTTCTACGGCGACGGCCGA
AAGTAAGGCCAGGTTGACCGACATGGCAATAGCTGCGACCAAGAGATCCTAT
Seq. ID NO: 23
>bkGPPS23
ATGTCACCGGCAGGAGCTCTGGCACCTCTAGCAGATTTCTTTGCTGCAGGCGGGAAAAGACTTAGGCC
GACTCTATGCGTGCTGGGGTGGCATGCGGCAGGTGGACAGACGCCTGCTTCAAGAGAGGTGGTGCAA
GTAGCTGCTGCGTTGGAAATGTTTCACGCGTTCGCTCTTATCCACGATGATGTAATGGATGACAGCGAC
ATCCGTAGGGGAGCGCCAACTTTGCACCGTGCGCTGGCAGGGCAGTACGCTGATCACAGGCCTAGGGC
ATTGACCGATAGATTGGGTGCCGGCGCCGCCATATTAATTGGCGACTTGGCTCTGTGCTGGTCAGACG
AGCTAATACATACGGCAGGTCTGAGGCATGATCAATTTGCCCGTATTTTGCCGGTGCTAGATATGATG
AGGACCGAGGTCATGTACGGCCAGTATTTGGATGTAACCGCCACGGGTCAACCTACCGCTGATATTGG
GAGGGCTCAAACGATCATCAGATACAAGACCGCAAAGTACACGATTGAAAGGCCGCTTCAGTTAGGT
GCGGAACTAGCTGGGGCCTCTACAGATGTGATAGACGCCTTGTCCGCCTACGCCGTTCCTTTAGGTGA
AGCGTTTCAATTAAGAGATGATCTATTAGGCGCATTTGGAGACCCCGTTGTAACCGGAAAATCCTCAA
CGGAAGACCTTCGTGAGGGGAAGCCAACGGTGCTTGTAGGCCTAGCATTGAGAGACGCAGCTCCAGA
TCAAGCTGACGTTCTTAGGAGGCTGCTTGGGAGGAGGGACTTAACTGAAGATCAAGCAACCCAAATTA
GGGCTGTTCTAACTGGCACTGGAGCTAGAGCCCAAGTGGAGAACATGATTGCACAACGTAGAGAGCG
TGTTCTGGCTCTGCTGGACACGAACACCGTGCTTGATGCGACTGCAGTCTTCCACTTACGTCAATTGGC
CGATTCCGCAACAAGAAGAACTAGT
Seq. ID NO: 24
>bkGPPS24
ATGTCAACGGTGTGCGCCAAAAAACATGTTCACCTTACTAGAGATGCAGCGGAGCAACTTCTGGCAGA
TATAGACAGGAGGTTGGATCAACTGTTACCAGTTGAGGGAGAGAGGGATGTCGTGGGTGCTGCTATGC
GTGAAGGGGCATTAGCCCCGGGCAAGCGTATTAGACCCATGTTGTTGTTACTGACAGCAAGGGACTTG
GGATGTGCAGTCTCCCACGACGGGTTATTGGATCTGGCCTGCGCGGTGGAGATGGTACATGCTGCGTC
TTTAATACTGGATGACATGCCCTGCATGGATGACGCAAAATTGAGAAGGGGGCGTCCAACCATTCATT
CTCACTATGGAGAGCACGTCGCCATTCTGGCCGCTGTGGCCCTATTGTCAAAGGCCTTTGGGGTCATAG
CAGACGCGGATGGCCTTACACCATTGGCGAAAAATAGAGCTGTCTCAGAGTTAAGCAATGCGATCGGT
ATGCAGGGGCTGGTACAAGGGCAGTTTAAGGATCTGAGTGAAGGCGACAAGCCGCGTTCCGCTGAGG
CAATTCTGATGACAAACCATTTCAAAACCTCCACCCTTTTCTGCGCGAGCATGCAAATGGCTAGTATTG
TTGCCAATGCTTCCAGCGAAGCTAGAGATTGTTTGCACCGTTTCAGCCTGGACTTAGGACAAGCATTTC
AACTGTTGGATGACTTGACAGACGGCATGACGGACACAGGAAAGGACAGCAATCAGGATGCAGGAAA
GTCTACGTTAGTGAATCTTCTTGGACCGCGTGCCGTGGAAGAACGTCTACGTCAGCATCTTCAACTTGC
TTCAGAGCATTTGTCCGCAGCGTGTCAGCACGGTCATGCTACCCAACATTTTATTCAAGCTTGGTTCGA
CAAAAAATTGGCCGCCGTCAGC
Seq. ID NO: 25
>rkGPPS1
ATGTCAGAGCTAGATAAGTACTTTGATGAAATAATTAAAAATGTCAATGAGGAAATTGAAAAATACAT
AAAGGGAGAACCCAAGGAATTGTACGACGCCTCAATTTACTTGTTAAAAGCGGGCGGGAAGAGGTTA
CGTCCGTTAATTACCGTTGCAAGTAGCGATCTTTTCTCTGGTGACCGTAAGAGAGCGTACAAGGCCGCT
GCTGCCGTCGAGATCTTACACAATTTTACGTTGATACATGATGACATAATGGATGAAGACACGTTAAG
AAGGGGTATGCCGACGGTACACGTTAAGTGGGGCGTCCCTATGGCAATACTAGCTGGAGACCTTTTGC
ACGCCAAGGCTTTCGAGGTTCTTAGCGAAGCGTTAGAGGGCTTAGATAGCAGGAGGTTCTACATGGGA
TTGTCCGAATTTTCTAAGTCCGTAATCATCATAGCTGAGGGACAGGCGATGGACATGGAATTTGAAAA
TAGGCAGGATGTTACAGAGGAAGAGTACCTTGAAATGATCAAGAAGAAAACTGCACAGTTGTTCTCAT
GTTCCGCGTTTCTTGGCGGGCTTGTAAGCAACGCAGAGGACAAGGATTTGGAGCTACTGAAGGAGTTC
GGCCTGAATCTTGGGATCGCGTTCCAAATAATTGATGACATTTTGGGTCTTACGGCTGATGAAAAAGA
ACTGGGAAAACCCGTCTACTCCGACATACGTGAGGGTAAGAAGACGATTCTTGTAATCAAAGCTCTAT
CCTTAGCTTCCGAGGCGGAGCGTAAAATAATCATCGAAGGTCTTGGAAGTAAAGACCAGGGGAAAAT
TACGAAAGCGGCGGAGGTCGTCAAAAGTTTATCACTGAACTATGCATATGAGGTGGCCGAGAAATACT
ATCAGAAGTCCATGAAAGCTCTATCCGCCATTGGAGGTAACGACATTGCTGGCAAAGCACTGAAGTAT
TTAGCGGAGTTTACCATTAAGAGGCGTAAGTAA
Seq. ID NO: 26
>rkGPPS2
ATGTCAACGCACGTACCCGCGAACGCAGTCCCCACAACTAACGGCTTGTCAATAATCCCTCCCGGTCT
GTCACTTCCGACAACTTTCGCCCCGTTGGTAGAACGTATACAAACTGTTGCTCACCTAGTAGAGACAG
CAATCGCCGAGGACTTGTCTGAAGTTACGCAACCTGAACTGCGTCAAGCGGTTCTACACCTATTCGAT
GGGAAAGGTAAAAGGCTTCGTCCATTCTTGGTGATTACGACCGCAGAGGCCGCGGGCGGCACTCTTGA
AGCCGCTTTACCACCCGCTTTGGCTGTTGAGTACCTTCACAACCTGAGTCTGATTCACGACGATATGAT
GGACGGGTCCCCTGAGCGTCACGGTAGACCAACCTTACATACTAGGTTTGGGCTAAACCTGAGTTTGC
TGGTAGGGGACTTACTTTATGCTAAAGCTGTTGAGCAAGCCTCTCGTATTAGGCATCACGCGCTAAGA
ATGGTGCACATTCTGGGGCAAACTGCCAAGCAGATGTGTTACGGTCAATTTGACGACCTGTACTTTGA
AAGGCGTTTGGATCTAACAATAGAGGATTATCTAAGGATGGCCGCAAGGAAAACTTCTGCCCTTTACA
GAGCTTCTTGCATTTTTGGGATGCTTACCGCAGACGCGGATGAGGCCGACCTTCAGGCGATGGCTACC
TTTGGAGAGAACATAGGAACCGCATTCCAGATCTGGGATGATGTATTAGACTTGCAAGCCGATCCGTT
ACGTTTAGGCAAGCCCTTAGGCCTTGACATTAGGGAAGGCAAAAAGACACTAATCGTTATCCACTTTC
TACAGCACGCTTCCCCTGCGGCGAGAAGGAGATTCCTGGAACTGCTAGGTAAACGTGATTTAAACGGA
GAATTGCCGGAGGCCATCGCGCTGTTGGAGGAGACGGGCTCAATAGCCTTTGCGCGTGACTTGGCGAT
AAGGTATCTAGTGGACGCGAAGCAGCACCTTTCCGTCTTGCCCGCCGGTCCGCACAGGAAATTATTAG
ACATGTATGCCGATTTCATGCTACAGAGAAGACATTAA
Seq. ID NO: 27
>rkGPPS3
ATGTCAACCTCAGAGACGAAGGAGGCGAGAGTGTTGGACGCAATTAGGGAGCGTAGAGATCTTGTAA
ACGCTGCTATTGATGAAGAACTTCCTGTCCAGGAACCCGAGCGTCTTTACGAGGCCACGAGATACATA
TTAGAGGCCGGAGGGAAGCGTCTGAGGCCCACAGTAACAACTTTAGCCGCCGAGGCTGTAACCGGAA
CCGAGCCTATGGGGGCTGACTTTAGGGCCTTTCCCAGTTTGGACGGGGATGACGTAGATGTTATGAGA
GCTGCAGTCGCAATTGAAGTCATTCAGAGCTTTACACTTATTCATGATGACATTATGGATGAGGATGA
CCTACGTCGTGGCGTCCCAGCTGTTCATGAGGCCTATGATGTCTCCACAGCTATTCTAGCTGGCGACAC
TCTGTACAGCAAGGCCTTTGAATTTATGACGGAAACTGGCGCAGACCCGCAGAACGGGCTGGAAGCTA
TGCGTATGTTAGCCAGCACGTGTACTGAAATCTGCGAGGGGCAGGCATTAGACGTTTCCTTTGAAAGC
AGGGACGATATATTACCCGAAGAGTACCTAGAGATGGTGGAACTAAAAACTGCCGTTCTTTATGGTGC
GTCAGCGGCAACACCTGCGCTTTTGCTGGGAGCTGATGAAGAGGTTGTTGACGCCTTATACAGATATG
GCATAGATAGCGGACGTGCCTTTCAGATACAAGATGACGTGCTGGATCTGACTGTTCCCAGCGAGGAG
CTGGGGAAGCAGAGAGGAAGCGATTTAGTAGAAGGTAAGGAAACATTAATCACACTTCATGCCAGAC
AACAGGGAATAGATGTAGATGGGCTTGTTGAGGCGGATACTCCTGCTGAAGTAACGGAAGCGGCAAT
CGAGGAAGCGGTAGCCACATTAGCTGAAGCAGGCTCCATAGAGTACGCTAGAGAGACAGCGGAAGAT
TTGACTGCACGTAGTAAGGGTCACTTGGAAGTTCTGCCTGAATCCGGTTCCCGTTCCCTGCTAGAGGAC
CTAGCTGATTACCTAATAGTAAGGGGCTACTAA
Seq. ID NO: 28
>rkGPPS4
ATGTCAGAAACCCTTACCCGTTATTTATCAGAGTTCAGACCGCTTGTTGATAAGAAGATAATGGAGGT
TCTTGAGGGAAGCCCTAAAGAATTATATGAAGCGGCCCGTCATCTGCCCTCTAAAGGAGGGAAAAGG
CTGCGTCCGGCTTTAGTATTGTTGGTCAACAAAGCCCTAGGTGGAGAGGTCGAAGGTGCGTTGCCCGC
TGCAGCCGCGGTCGAACTTTTACACAACTTCACACTTGTCCACGATGACATAATGGATCGTGACGAGT
TGCGTCGTGGTGTTCCGACTGTGCATGTTTTGTACGGCGAATCCATGGCGATTTTGGCTGGTGACTTGT
TATATGCGAAAGCATACGAGGCGCTGCTACAGTCCCCGCAACCACCCGATCTTGTTAAGGAAATGACC
GAAGTGTTAACTTGGTCTGCCGTGACAGTTGCCGAGGGTCAAGCCATGGATATGGAATTTGAAAAGCG
TTGGGACGTGACCGAGGAAGAATATTTGGAGATGATAGAAAAGAAAACAGGGGCACTTTTTGGAGCT
TCCGCAGCTCTGGGGGCGCTGACCGCAAATAAGCGTGAGGTCAAAGATCTGATGAAAGAGTTCGGGC
TAATTTTAGGGAAGGCTTTCCAGATAAAGGACGATGTGCTTTCCCTTTTAGGTGATGAAAAAGTTACC
GGAAAACCAAAGTATAATGATCTTAGGGAGGGGAAGAAAACCATCCTGGTGATTTATGCGTTGAGAA
ATTTACCCCGTGATGAAGCAGAAAGAGTAAAGTCAGTGCTTGGCAGAGAAACCTCCTACGAAGCGTTA
GAAGAAGTTGCAGAACTAATTAAGAGAAGTGGTGCTCTGGATTACGCTATGAAACTGGCTGAAGAGTT
CGAGAAAAGAGCGTACGAGATATTAGAAACTGTCAGGTTTGAAGACGAAGAGGCGATGAGGGCCCTA
AAAGAGCTGGTCGATTTCGCAGTTAAGAGGGAATATTAA
Seq. ID NO: 29
>rkGPPS5
ATGTCAGGGAAACAATTCAACCTGCTGAGGGAAAAATATCTTCCGCAAATCGAAAGGGAGATTAAGA
AATTTTTCGAGGAGAAAATCAGCACACAGAAAGACGAGGTCATTGTCAGATACTACGAGGAACTGTCT
TCATACGTACTTAGAGGAGGGAAAAGGTTCAGGCCGCTTGCCCTTATCTCATCTTATTATGGGAGCGG
CTCAAAGCATGAAGGTAACATTATTAGGGCATCAATAAGCGTTGAGCTTCTACACAACAGCTCTTTGA
TACACGACGATATAATGGACGAAAGTCCAAAGAGAAGGGGTGGTCCAAGTTTTCATTATCTGATGGCA
AATTGGAGTAGGCTATCCCCCAGAACGCCTCCACCAAGGAACCCCGGAATCTCTCTAGGCATTCTGGG
CGGGGACTCCCTAATCGAGCTAGGCTTAGAGGCTCTACTTGAGAGTGGATTTCCAAACGAGATCATTG
TTAAGGCCGCTAGTGAATATTCCGTTGCATATAGAAAGCTGATTGAAGGGCAGCTACTTGACTTATAT
CTGTCTACAGTTACCATGCCTACTGAAGAGGAAGTACTGCGTATGCTTTCTCTAAAGACCGGGACTCTA
TTTAGCGCATCCTTAGTTATGGGTGGCATGTTAGCCGGTGCATCAGAGGATATGTTACACTTTCTAAGG
TCTTTTGGTCAGAGAGTTGGGGTAGCATTCCAGTTACAAGATGACATTCTGGGACTTTACGGGGACGA
AGCCGTCATCGGGAAACCAGCCGATTCTGACATAAAAGAAGGTAAACGTACGCTATTGGTGGTGAAA
GCCTGGGAACTGTCCGATGAGGCCACCAGGAAGAAGCTACTTTCCATACTTGGCAACCCCAATATCAG
TGCTGCAGATCTAAACTACGTCCGTGAGGTAGTTAAAGAGCTAGGAGCCTTAGACTACACTCGTAAGA
CCGCCCTTAATCTACTGAAAGAGAATGAGAAAGATATTGAGTTCAACAAACACTTGTTCGAGGAATCA
TTTGTAGAGTTTTTGAAGGAGCTGAACGAAATTGTAATAGCGAGGTCATTCTAA
Seq. ID NO: 30
>rkGPPS6
ATGTCATCAAATATCAACGAAGATGTCGGGAAAGTTCTTGGTCAGTATAGTAAAGACATACACAAGGA
AATCGGAAACACACTGAGCAACATTGGACCCGAGGATCTAAGAGAAGCGAGTATTTACCTGACCGAG
GCAGGTGGTAAAATGCTACGTCCCGCTCTGACCGTGCTTATCTGTGAAGCAGTAGGCGGCACGTTCAG
CAGCTGTATAAAAGCAGCTGCAGCGATAGAATTGATCCATACATTCAGCTTAATTCACGACGACATAA
TGGATAAGGACGATATGAGAAGAGGTAAGCCGTCAGTCCACAAGGTGTGGGGCGAGCCGGTTGCCAT
ACTTGCGGGTGACACCTTATTTTCTAAGGCTTATGAGTTGGTGATCAACAGTAAGAATGAAATAGATT
CTTCTAACCCTGAAGAGTGTCTGAACAGGGTGAACCGTACCTTGAGCACCGTTGCGGACGCGTGTGTT
AAAATATGTGAAGGGCAGGCACAGGATATGGGCTTCGAAGGTAATTTCGATGTATCTGAAGAGGAGT
ATATGGAAATGATCTTCAAAAAGACCGCTGCTCTGATAGCGGCAGCAACCGAATCCGGGGCCATAATG
GGTGGTGCGAACGAAAAGATTGTGAGCGATATGTATGACTATGGTAAATTAATAGGTCTAGCGTTCCA
AATACAAGACGACTATCTGGACCTTGTCAGCGACGAAGATAGCTTAGGTAAACCCGTCGGTTCCGACA
TCGCAGAAGGAAAGATGACAATCATTGTAGTTAATGCATTAAACAGGGCGAACCCAGAGGACAAAAA
GCGTATCTTGGAAATTCTTCGTATGGGCAATGAGTCAGGTAACTGCGACCAAGTCTATGTGGATGAAG
CAATATCTCTATTCGAGAAATACGGGAGTATACAATACGCCCAGAATATTGCTTTGGCCAACGTCAAA
AAGGCCAAGCAACTGCTTGAAATACTACCGGAATCCGAGGCTAAGCATACTCTTTCCCTAGTTGCCGA
CTTTGTTTTATATAGACAAAACTAA
Seq. ID NO: 31
>rkGPPS7
ATGTCATCAGATTTGAAGACCTACCTAGAGAAGACGGCGGAACAGGTCGATATCGCATTGGAAAGAA
ACTTTGGTGACGTTTTCGGAGACCTTTATAAGGCTTCAGCGCACCTACTATTAGCAGGGGGAAAGCGT
TTACGTCCCGCCGTACTATTGCTGGCGGCTAATGCGGTTAAACCAGGACGTGCAGACGACCTAATTAC
GGCTGCCATAGCCGTTGAAATGACACACACGTTTTACTTGATACATGACGATATAATGGACGGTGATG
TTACCAGAAGGGGTGTTCCCACGGTTCATACTAAATGGGACGAACCAACGGCCATACTAGCAGGGGA
CGTATTGTACGCCAAGTCATTTGAGTACATCACGCACGCTTTAGCGGAAGATCGTGCTCGTGTGAAGG
CTGTTACACTATTAGCCCGTACTTGCACGGAAATCTGCGAAGGTCAACACCAAGACATGGCCTTTGAG
CAAAAAGGCGCTGAAGTAGAGGAAGCGGACTACATTGAGATGGCTGGTAAGAAAACAGGTGCTCTAT
ATGCCGCCGCTGCCGCTATCGGTGGAACTCTTGCCGGTGGAAACGCAATGCAGGTGGACGCACTTTAC
CAATATGGGATGAATGCGGGAATTGCTTTTCAGATCCAAGATGATCTGATAGACCTTCTAGCGCCTCC
AGAAACCTCCGGAAAGGACAGGGCATCTGACCTTAGGGAGGGGAAGCAAACATTGATCGCCATTATA
GCCAGGGAGAAAGACCTAGATCTTTCAAAGTACAGACACACGCTGACAACGACAGAGATTGACGCTG
CAATCGCAGAACTGGAAGGTGCAGGTGTAGTTGACGAGGTTAGGAGGGCTGCGGAAGAAAGAGTGGC
GACCGCTAAGAGAGCTTTATCCGTGCTGCCGGAGAGCATGGAGAGGACCTACCTAGAGGAGATCGCT
GATTACTTCCTGACCAGATCATTCTAA
Seq. ID NO: 32
>rkGPPS8
ATGTCAGATCTTATCGACGAGCTGAAAAAGCGTTCAACACTTGTAGACGAGTCTATACAGGAATTTTT
GCCCATCGATCACCCTGAGGAGCTGTACCGTGCAACGAGGTATTTACCCGACGCTGGTGGTAAACGTC
TGAGACCAGCTGTGCTTATGTTAAGCGCAGAAGCAGTGGGCGGCGACAGTGACTCCGTATTGCCTGCT
GCGGTTGCACTTGAACTAATCCACAACTTCACCTTGATTCACGATGACATCATGGATAGAGACGACAT
AAGGAGGGGGATGCCCGCCCTTCACGTAAAGTGGGGAACTGCAGGTGCCATCTTGGCCGGTGACACA
CTTTACTCAAGGGCCTTCGAGATCATATCAAAAATGGATGCTGATCCTCAAAAATTGCTGAAGTGCGT
TGCTTTGCTAAGCAGAACCTGCACTAAGATCTGTGAGGGACAGTGGTTGGATGTGGACTTTGAGAAAA
GAGATATCGTTGATGTGGATGAATACCTAGAAATGATTGAAAATAAGACGTCAGTCTTGTATGGTGCT
GCTGCTAAGGTTGGAGCGATTCTGGGAGGCGCGAGTGATGAGGTTGCTGATGCTATGTATGAGTTCGG
TAGGCTAACGGGCATTAGTTTCCAGATCCATGATGACGTTATAGACCTGGTTACCCCTGAGGAGATTCT
TGGTAAGAGTAGAGGATCTGACCTGAAAGAAGGGAAAAAGACATTAATTGCACTTCACGCTCTAAAC
AATGGTGTAGAATTGGAATGTTTTGGTAAAGCAGACGCCACGCAAGACGAAATAAACAATGCTGTCG
CTAAATTGGAAGAGAGTGGTACTCTGGCTTATGTCCGTGAGATGGCTGACAACTACTTAGAAGACGGG
AAGAGTAAGCTGGACTTATTAGAAGATAGTCCCGCGAAAGAAACCTTAATCGAGATCGCAGATTACAT
GGTTAGTAGAGAATACTAA
Seq. ID NO: 33
>rkGPPS9
ATGTCAGATCTTATTGAAGAAATTAAGAAACGTTCATCTCACGTAGATAAAGGTATAGAGGAGTACTT
GCCAATCGATAAGCCCTATGAATTATATAAAGCTGCAAGATATCTACCAGACGCCGGAGGAAAGCGTC
TAAGACCGGCAACTGTAATACTTGCTGCCGAGGCCGTCGGGAGCGACCTAGAGACTGTACTTCCTGCT
GCAGTAGCGGTGGAACTTGTTCATAATTTTTATTTGGTCCACGATGATATCATGGATCGTGATGATATA
AGAAGGGGTATGCCTGCCGTTCACGTGAAATGGGGCGAGGCAGGCGCCATTCTGGCTGGCGATACGCT
GTATTCAAAAGCCTTTGAGATATTAACCCACGCTCCCGCAGAGGCCCCGGAGAGAAACCTAAAGTGTA
TTGATATCTTATCAAAAGCGTGTCGTGATATTTGCGAGGGGCAATGGATGGATGTAGAGTTTGAGAAC
AGGGATGACGTAACTAAAGAGGAATATCTGGAAATGATCGAGAAGAAGACTGGAGTTTTATACGCCG
CGTCTATGCAGATAGGTGCAATCCTGGGTGGCGCGCCTGAAGAGGTGAGTGACGCTTTTTACGAGTGC
GGCAGACTAATCGGCATAGCATTTCAAATTTATGATGACGTAATTGACATGACTACACCAGAAGAGGT
TTTAGGGAAGGTTCGTGGTTCAGACCTTATGGAGGGTAAGAAAACACTTATAGCAATACATGCCTTGA
ACAAGGGTGTCGAATTAAAGATTTTCGGTAAGGGTGAAGCGACCACTGAGGAAATTAATGAAGCAGT
TCACCAGCTTGAAGAAGCTGGCAGTATAGATTATGTTAGAGATTTAGCCCTTGACTATATAGCAAGAG
GAAAGGAATTGTTAAACGTAGTTGAAGACTCCGAGTCCAAGACCATACTTAAAGCTATAGCAGACTAT
ATGATAACTAGGTCTTATTAA
Seq. ID NO: 34
>rkGPPS10
ATGTCAATTGAGGAAATATTACAAAAGAAGGCCAAATTGGTAGACGAAAGCATACCTAAGTTTCTTCC
TATAACGCCGCCGGACGAACTGTATAAAGCCATGAGGCACCTGTTAGATGCTGGCGGGAAGAGACTA
AGGCCTTCAGCTTTACTACTTGCCAGTGAGGCCGTAGGCGGTAAACCCGATGATGTCCTGCCTGCGGC
TGTTGCGGTTGAGTTAGTCCACAACTTCACATTGATACATGATGACATCATGGACGAGGCGGATCTGA
GAAGAGGTCTTGCAACAGTACACAAGAAATGGGGAGTACCAAGAGCTATAATTGCGGGAGACGCACT
TTACTCTAAGGCATTTGAGATTCTATCTTGCACAAAGAGCGAACCCCAGAGGCTGGTTGAAAGTCTTG
AGCTACTGAGTAAAACATGCACGGACATCTGCGAGGGCCAGTGGATGGATATGAATTTCCAGACAAG
AAAAGATGTAACCGAAGAAGAATACATGCGTATGGTTGAAAAGAAGACCGCGGTGTTGTTTGCCACT
GCACTGAAATTGGGGGCGGTCCTGAGCGGTGCCAATAGGGAACACGTAAGAGCCCTATGGGACTTCG
GCAGGCTAACTGGAGTCGGTTTTCAAATATACGATGATGTGATAGATCTAATAACACCAGAAGAGATA
CTGGGTAAAGCGCAAGGCGGCGACATAATAGAGGGTAAGAGGACCTTAATTATCATCCACGCTCTAA
GTAAAGGGATTTCTATTGACGCCTTAGGCAAGTGCAACGCTACTAGGTCTGAGATCAGTGCAGCATTA
ACCACGCTAAAGGAATCCGGATCTATTGATTATGCAATGAACAAAGCACTAAGTTTCGTCGATGAAGG
CAAAGCAGCTCTAGCGATGCTGCCTGAATCAGAGGCGAAAAACATTCTAACTCGTTTAGCCGACTATA
TGATTGAGCGTAAATATTAA
Seq. ID NO: 35
>rkGPPS11
ATGTCAGAAGCCGATATGAGCGACTTATCAGCCTACCTTAAATCTGTGGCACAGCAAATAGACGGTAT
GATCGAGAAAAACTTTACCCATGCAGGGGGAGAGTTGGACAGAGCCTCTGCACACCTATTGAGCGCA
GGAGGGAAACGTCTGAGGCCCGCCGTGGTCATGCTGAGTGCAGACGCGATCAGACATGGCTCAAGTA
AGGATGTAATGCCCGCCGCCCTTGCTTTGGAGGTCACCCATACATTTTACCTAATACATGATGATATCA
TGGATGGAGATAGTCTGAGACGTGGAGTTCCAACTGTTCATACGAAGTGGGATATGCCAACAGGTATT
CTTGCCGGAGACGTCCTTTATGCTAGGGCATTCGAGTTCATTTGCCAGAGTAAGGCTGATGAAGGCCC
TAAAGTGCAAGCCGTAGCTTTGTTGGCAAGGGCTTGCGCCGATATATGCGAAGGTCAACACCAGGACA
TGTCATTCGAACATAGGGCAGATGTAACTGAAGAAGAATACATGGCTATGGTGGCTAAAAAGACAGG
CGTATTGTACGCAGCGGCGGCTGCTATCGGCGGAACACTGGCGGGAGGGAACCCGGAACAGATCAGG
GCTTTGTACCAGTTTGGGTTAAATACAGGAATCGCCTTTCAAATACAAGATGATCTAATTGATCTTCTG
ACCCCCACTGAGAAGAGTGGAAAAGACCAGGGTAGCGACCTGAGGGAGGGAAAGCAAACTCTGGTCA
TGATCATTGCAAGGCAAAAGGGTGTGGATCTATTGAAATATAGACACGAACTTTCTCCTGCTGACATT
AAAGCGGCAATCCAGGAATTAACTGATGCGGGTGTCATTGACGCAGTTAAGAAGAAGGCGGCTGATC
TAGTGGCAGATTCCAATAGGTTGCTTATGGTCCTTCCGCCCACTAAGGAGAGACAGTTGATTATGGAC
GTAGGGGAGTTCTTCGTTACGAGGTCTTTTTAA
Seq. ID NO: 36
>rkGPPS12
ATGTCAGAGTTGATTGAATATCTGGAAAAGGTAGGGAACCAAGTCGATCGTTTAATCGATAGGTATTT
TGGAGATCCTGTGGGTGAACTAAACAAAGCGAGTGCGCACCTGCTTACTGCCGGTGGCAAGCGTCTTC
GTCCCGCGGTAATGATGCTGGCTGCAGACGCTGTAAGGAAGGGCTCTTCTGACGACTTGATGCCGGCT
GCTATCGCTTTAGAATTGACTCATTCATTTTACTTAATCCATGACGATATAATGGACGGCGACGAGGTT
AGACGTGGAGTCCCAACTGTTAACAAAAAGTGGGACGAGCCAACCGCCATTTTAGCGGGGGATGTGC
TTTACGCGAGGGCTTTTGCATTCATATGTCAAGCCCTTGCAATGGACGCTGCTAAACTGAGAGCAGTTT
CCATGTTGGCGGTTACGTGTGAGGAAATTTGTGCTGGACAGCATCTTGACATGGCCTTCGAAGATAGA
GATGATGTTTCAGAAGAGGAATATCTTGAAATGGTCGGGAAGAAAACTGGCGCTCTTTATGCAGCATC
AACTGCTATGGGTGGAGTCCTTGCGGGTGGTTCCCAGCCGCAAGTAGATGCGCTTTACCGTTACGGCA
TGAACATCGGCGTTGCGTTTCAAATTCAGGATGACCTGATTGATCTTCTGGCGTCTCCCGAAAGGTCAG
GTAAAGATAGAGCTAGTGACATACGTGAAGGAAAGCAAACACTGATTAACATAAAGGCGAGGGAGCA
CGGTTTTGACTTAGCCCCATACAGAAGACGTTTAGACGATGCTGAGATAGACGACTTAATTCAGCAAT
TAACAGATAATGGCGTTATTGGCGAAGTAAAAGCCACTGCGGAAGGACTGGTCACTTCTGCTGGTAAG
ATCCTTGCTATTTTGAAACCATCAGACGAAAAGGACTTATTGATAAGTATAGGTACCTTTTTCGTTGAA
CGTGGCTACTAA
Seq. ID NO: 37
>rkGPPS13
ATGTCAAAAGATACGACGAAGATTGAAGTGGAGAACTACATTAATAAAGTGAATAACCATCTAATTTC
ATTTCTTAGTGGGAAGCCACTTCAATTATATCAAGCAAGCACGCATTACCTGAAGTCAGGCGGAAAGA
GATTAAGACCGATAATGGTTATCAAATCCTGTGAAATGTTCGGTGGGACACAACAGGATGCACTACCT
GCGGCAGCAGCCGTCGAGTTTATTCACAACTTCTCCCTAGTGCACGATGATATTATGGATAACGATGA
CCTTCGTCACGGTATTCCAACTGTGCATAAGAGCTTTGGATTACCGCTTGCGATCCTTAGTGGTGACAT
TTTATTTTCCAAGGCTTTTCAAATACTTAGTATAACCAACGTAAACTCAATTAAAGATTCCAGCCTTCT
ATCAATGATAAGGAGGTTGTCCCTAGCCTGTGTAGATATCTGCGAAGGTCAGGCTAAAGATATACAGT
TCAGCGAATGTGAGACTTTTCCATCAGAAGAGGAGTATCTTGAAATGATCTCAAAGAAGACGGCAGCT
CTTTTTAACGTGTCCTGTTCACTAGGCGCGTTATCAAGTAGGAATGCCACAGAAAAAGACGTCAATAA
TATGAGTGACTTTGGCAAAAATTCCGGTATTGCGTTCCAGTTGATTGACGACCTGATAGGAATCGCGG
GACACTCAAAAGAGACGGGCAAAGCCGTAGGCAATGATATTCGTGAAGGGAAAAAGACATATCCGAT
CCTGTTATCCATCAAGAAGGCGAGCGAGTTGGAGAGGGCGCACATTCTAAAAGTGTTCGGCAAAGGG
CAGTGCGATAACATGAGCTTAAAGAAGGCAATAGACGTCATCTCTAGCTTGCAGATAGAGAAGATCGT
CAGGAAATCCGCGATGGCATATATCGAAAAGGCGATGGAGGCCCTGGTTAATTACGAGGATTCTGAA
CCGAAGAAGATATTACAGGAGTTGTCATCTTACATAGTAGAGCGTTCTAAATAA
Seq. ID NO: 38
>rkGPPS14
ATGTCACTGCAAGACTACTTTAACGAAGTAATCAATCAGGTGAACAAAACCATTGAGAAATACCTAAG
TAACGCCCCGAGCGGAACGAGTAGCTTATACGAGGCCTCAAAACATTTATTTTCTGCTGGAGGAAAGA
GACTGAGACCTCTAATCTTGGTAAGCTCCTGTGACTTTCTGGGTGGCGACCGTTCACGTGCCATCCTGG
CAGGATCAGCCATCGAAACTCTACATACATTCACATTGATCCACGATGACATCATGGACCATGATTTTC
TGAGAAGGGGCTTGCCTACTGTACATGTCAAATGGGGTGAATCTATGGCTATCTTGGCTGGCGATCTA
CTTCACGCTAAAGCATTTGAAATGCTAAATGACTCACTAGAAGGGGTGAATGAGACGCTACACTACGA
AGTGATGAAGACATTTATTAATTCTATCGTGGTAGTGAGTGAAGGGCAGGCCATGGACATGCAGTTTG
AGGGGCGTAATGACGTGACAGAGGAGGACTACCTGGAAATGGTAAAGAAGAAGACTGCTTATCTAAT
CGCCACTAGCTCTAAAATTGGTTCATTAATTGGCGGTGCGGGCCCAGATGTCGCCGACAAATTCTTTCA
CTTCGGGATTTATCTTGGCATAGCCTTCCAGATTGTTGATGACATCATTGGCATAACATCAGACGAGGC
TGAGCTGGGCAAGCCGTTATTTTCTGACATAAGGGAAGGAAAAAGAACACTTCTGGTAATCAGGACGT
TAAAGGAAGCCGAGTCACGTGAGCTTGAAGTTCTTAAACAAGTTTTGGGCAATAAGAATGCCAGTACC
GACCAACTGAAAGAGGCCTCCCAAATCGTCAAGAAGCACTCTTTGGAGTACGCATACAGTTTAGCTGA
GGAGTATAGATCCAGAGCTATCTCATCACTTGATGGCATACAGCCGCGTAATCAAGAGGCTTATGAGG
CCCTGAAGTTCGTGAGCGAATTTACGTTAAAGAGGAAAAAGTAA
Seq. ID NO: 39
>rkGPPS15
ATGTCATCTTTTAATTCAATCTCCAAAACAGCAAAGAAGGTGAACTCATTTTTATTGTCTAGCTTACAC
GGAAACCCTGAGGAGATTTACAAAGCTGCGAGCTACTTGATTGAATACGGCGGAAAAAGGTTACGTC
CGTACATGGTAATAAAATCTTGTGAAATACTTGGAGGCACAATCAAGCAGGCATTACCATCTGCAGCC
GCAATCGAGATGGTCCATAACTTTACCCTAATACACGACGACATTATGGACAATGACGAAATTAGACA
CGGCGTGAGCACGACCCATAAGAAATTCGGCATCCCCGTAGGGATTCTTGCGGGGGATGTGCTGTTTT
CCAAAGCGTTCGAGACCATTTCACATGGAGATCCTAAGATGCCCAAAGACGTCAGATTAGCCTTAGTG
TCAAACCTTGCCAAAGCGTGTACTGATGTGTGCGAAGGCCAAGCTCTTGACATTATGATGGCCAAATC
ACAGAAGATTCCTACTGAGGAGCAGTATATTATGATGATCGAAAAGAAGACAAGTGCATTGTTCGCAG
CGGCGTGTGCGATGGGCGCAATTAGTGCAAACACAAAGACGAGGGACGTCACAAACTTATCTAGCTTT
GGCAAAAACCTGGGAGTTGCGTTTCAAATCGTAGACGATTTGATTGGAATTATTGGTGATTCTAAGAT
AACCAAAAAGCCGGTCGGGAATGATTTAAGAGAGGGCAAAAAGAGTCTGCCAATTTTGTTGGCCATT
AACAAAGTCTCTGGTAAGAAGAAGGAAATTATCCTGAATGCCTTTGGTAATTCCGCGATATCAAAGAA
AGAGCTTGAGAACGCAGTGAGGATTATTAGCTCCATGGGGATAGAAACGGCTGTTAGAAAGAAGGCC
ATACAATACTCCAATGCCGCCAAAAAGAGCTTGAGCAACTATAAAGGGAGTGCTAAAAATGAGCTGC
TTTCCTTACTAGACTTCGTGGTCGAGAGAAGCCAGTAA
Seq. ID NO: 40
>rkGPPS16
ATGTCAGGCAAATATGATGAGTTATTTGCCCAAGTGAAGGCTAAGGCGAAAGACGTGGACGCCGTAA
TTTTTGAGCTAATACCCGAAAAGGAGCCCAAGACGTTGTACGAAGCTGCGAGACATTATCCTTTAGCT
GGAGGCAAAAGGGTTCGTCCCTTTGTTGTGTTGAGGGCAGCCGAGGCGGTTGGTGGCGACCCCGAAAA
GGCTCTGTACCCGGCTGCCGCAGTAGAATTTATTCATAATTATTCTCTGGTTCATGATGACATCATGGA
TATGGACGAACTAAGACGTGGCAGGCCCACTGTGCATAAGTTATGGGGCGTCAACATGGCCATCCTAG
CTGGCGACTTGTTATTCAGTAAAGCATTCGAGGCCGTTGCAAGAGCTGAAGTAAGCCCTGAAAAGAAG
GCTAGGATATTAGACGTTTTGGTCAAGACCTCAAATGAATTGTGTGAGGGTCAGGCCCTGGACATTGA
GTTTGAAACCAGGGATGAGGTAACAGTTGATGAATATCTTAAAATGATTTCTGGAAAGACAGGTGCGT
TGTTCAATGGGTCTGCCACCATCGGAGCCATCGTAGGAACGGACAACGAGAAGTACATTCAAGCACTG
AGTAAGTGGGGGAGGAATGTCGGTATCGCCTTTCAAATCTGGGACGACGTTCTTGATCTTATCGCAGA
TGAAGAAAAACTAGGGAAACCCGTTGGCAGTGACATAAGAAAAGGGAAGAAGACGTTAATTGTGAGC
CACTTTTTCCAGCACGCGAATGAAGAGGACAAAGCCGAATTTTTGAAGGTATTTGGTAAGTACGCGGG
GGATGCTAAGGGAGACGCGCTTATACATGATGAAAAGGTCAAAGAGGAAGTGGCCAAGGCGATCGAA
CTTCTTAAAAAGTATGGATCTATCGATTATGCCGCTAATTACGCTAAGAACTTAGTTAGAGAGGCTAA
CGAGGCGCTAAAGGTGCTACCGGAGAGCGAGGCGAGGAAGGACCTTGAATTACTAGCCGAATTTTTA
GTTGAAAGAGAATTTTAA
Seq. ID NO: 41
>rkGPPS17
ATGTCAGATATTATAAGCAGGTTCTCCGAAAAGATCGACGCCGTTAATTCTGCAATAGACAAGTTCCT
AAGGATACGTGAACCTAAAAGACTGTACTCTGCGACGAGACACCTTCCACTTGCAGGAGGCAAGAGG
CTACGTCCTATTCTGGCAATGTTATCAACAGAAGCCGTAGGCGAGGACTGGAAGAAAACAATACCCTT
TGCGGTGTCCTTAGAACTTCTTCATAATTTCACTCTGGTGCACGATGATATAATGGACCGTTCCGATCT
TAGAAGAGGAATCGAAACAGTTCACGTGAAGTTCGGCGAACCTACTGCTATACTTGCGGGAGATATAC
TTTTCGCTAAGTCCTTCGAGGTGCTTTACGAATTAGATATTGACGACGCAATCTTCAAAACTGTTAATA
GATTACTGATAGATTGTATTGAGGAAATATGCGATGGACAGCAGATCGATATGGAATTTGAGTCACGT
AAATACGTCAGCGAAGAGGAATATCTTGAGATGATTGAAAAGAAAACAAGCGCACTGTTTAGTTGCG
CGACAACGGGTGGTGCCATTATCGGGGACGGGAATAACCGTGAAGTCGATTCTCTTTCCTTGTACGGG
CGTTTCTTCGGTCTAGCTTTCCAGATTTGGGACGACTACTTGGATATCGCGGGGGAGGAGGGGGAATT
TGGGAAGAAGATAGGAAACGACATTAGGTGTGGCAAGAAGACCCTAATGATCGTTCACGCGACTAAG
AATGCTGATGGGAGAGAGAAGGAAACGATCTTCTCTATTCTTGGAAAGAAGGATGCAACGGATGAGG
AAATTAACGAGGTAATGGAGATCTTAACAAAGTCTGGAAGCATTGACTACGCGAAGAAAAAGGCGTT
ACACTTTGCCGAAAAAGCAAAAGAACAACTTAGGGTGTTACCAGATTCAAGGGCCAAGAGGGATTTG
ATTGAATTAGTCGATTTCGCCATTAGCAGAGAACGTTAA
Seq. ID NO: 42
>rkGPPS18
ATGTCACTTATTGACCACTATATTATGGATTTTATGTCAATTACACCAGATCGTCTGAGTGGTGCTTCC
CTTCATTTGATTAAAGCGGGTGGAAAAAGGCTAAGGCCTTTGATTACCTTGCTAACAGCGAGGATGCT
TGGAGGTCTGGAAGCAGAAGCGAGGGCGATACCGCTGGCGGCATCCATTGAAACGGCCCATACCTTCT
CCTTGATTCACGATGACATTATGGATAGAGATGAGGTGCGTAGAGGCGTACCAACAACGCACGTTGTC
TATGGAGATGACTGGGCGATTCTGGCAGGGGATACCCTTCATGCAGCTGCATTTAAAATGATCGCCGA
TTCCAGGGAGTGGGGTATGAGTCACGAACAGGCCTATAGGGCTTTTAAGGTATTATCAGAGGCGGCAA
TACAGATATCAAGAGGTCAGGCATACGACATGTTGTTCGAAGAGACTTGGGATGTAGATGTCGCTGAC
TACCTGAACATGGTAAGGCTGAAGACGGGAGCTTTGATAGAAGCGGCAGCCAGGATCGGCGCTGTAG
CAGCAGGGGCTGGATCAGAGATTGAGAAAATGATGGGCGAAGTTGGGATGAACGCGGGTATAGCGTT
CCAGATTCGTGATGACATTCTTGGCGTCATCGGAGATCCCAAAGTCACTGGAAAGCCCGTCTACAACG
ACCTTAGGAGAGGCAAAAAGACCCTGTTGGTAATCTATGCTGTAAAAAAAGCGGGTAGGCGTGAGAT
TGTTGACCTTATAGGCCCTAAGGCGTCAGAGGACGATTTAAAGAGGGCAGCTAGTATCATTGTTGACA
GTGGTGCTCTAGATTACGCGGAATCAAGAGCTAGGTTTTACGTGGAGAGAGCTAGGGATATATTGTCT
CGTGTCCCCGCAGTAGACGCGGAATCCAAAGAACTGCTTAATTTGTTACTGGATTACATAGTGGAACG
TGTCAAATAA
Seq. ID NO: 43
>rkGPPS19
ATGTCAATCTCAGAAATAATTAAGGATAGAGCGAAGCTAGTGAATGAGAAGATCGAAGAACTGCTAA
AGGAGCAGGAGCCGGAGGGGTTATATCGTGCAGCGCGTCATTACTTGAAGGCTGGCGGGAAGAGATT
GAGACCCGTCATAACCCTGTTGTCAGCGGAAGCCTTGGGTGAGGACTACAGGAAGGCGATCCACGCA
GCGATTGCTATTGAGACTGTTCACAACTTCACCCTAGTCCATGATGATATTATGGATGAGGATGAAAT
GAGAAGGGGCGTGAAGACTGTTCACACATTGTTTGGGATTCCCACAGCTATCTTAGCTGGAGACACAC
TATATGCCGAAGCATTCGAAATCTTAAGCATGTCTGATGCGCCGCCAGAAAACATCGTTAGGGCCGTC
TCTAAACTTGCGAGAGTTTGTGTTGAGATTTGCGAGGGCCAATTCATGGACATGTCCTTCGAAGAACG
TGACAGTGTCGGCGAGAGTGAGTACTTGGAGATGGTCCGTAAGAAGACTGGCGTGCTTATAGGTATAA
GTGCAAGTATCCCCGCAGTACTGTTCGGTAAGGATGAATCTGTGGAAAAAGCCTTATGGAATTATGGG
ATTTACTCAGGGATTGGGTTCCAGATCCACGATGACCTGCTGGATATTTCAGGGAAAGGTAAAATAGG
CAAGGACTGGGGTTCCGATATACTAGAGGGCAAAAAGACACTAATAGTAATTAAGGCCTTCGAAGAA
GGAATCGAACTAGAGACGTTTGGAAAGGGCAGGGCTAGTGAAGAGGAGTTAGAGAGGGATATTAAAA
AGTTATTCGACTGCGGAGCTGTCGACTACGCTAGGGAAAGGGCCAGAGAATATATTGAGATGGCGAA
AAAAAACTTAGAGGTCATAGATGAAAGCCCATCTAGAAATTACCTGGTTGAGTTAGCAGACTACCTGA
TTGAAAGGGATCATTAA
Seq. ID NO: 44
>rkGPPS20
ATGTCATCCGAACGTCATCAACAGGTAGAGGACGCAATCGTAGCACGTCGTGATAGGGTTAATGACGC
ACTACCTGAAGATCTGCCAGTGAAGAAGCCTGACCACCTATACGAAGCTAGTAGGTATCTGCTTGATG
CCGGGGGGAAAAGGTTGAGGCCTACAGTTCTGCTGCTGGTGGCAGAGTCCCTTCTTGATGTGGATCCT
CTTACGGCAGACTATCGTGATTTTCCCACCCTAGGGGGCGGCCAGGCAGACATGATGTCTGCAGCTCT
TGCCATAGAGGTGATTCAAACTTTTACTCTAATACATGATGATATTATGGACGACGACGCTTTAAGGC
GTGGGGTTCCCGCAGTTCATAAAGAATACGACTTGAGCACAGCAATCTTAGCCGGAGATACATTATAT
TCCAAGGCTTTTGAGTTCTTGCTAGGGACAGGTGCAGCGCACGAAAGAACGGTCGAGGCAAACAAGA
GATTAGCGACGACCTGCACACGTATTTGTGAGGGGCAGAGCTTGGACATTGAATTTGAACAGCGTGAC
GTTGTCACACCGGAAGAGTACCTAGAGATGGTGGAGCTGAAAACTGCAGTATTATATGGAGCGGCGG
CTAGCATACCAGCTACATTATTAGGAGCGGATGCCGAGACCGTCGACGCGTTGTATAACTACGGACTT
GATGTTGGAAGAGCTTTTCAAATACAAGACGATTTGTTAGATTTAACAACACCATCCGAAAAATTGGG
TAAGCAAAGAGGGTCCGATCTGGTCGAAAACAAACAAACGCTTGTTACTCTGCATGCCAGACAACAA
GGAGTGGATGTCGGCGACCTAATTGATACCGATTCTGTAGAGGCTGTAAGTGAAGCAGAAATTGATGC
TGCAGTCGAGAGACTGAGGGAGGTCGGTTCTATTGAATATGCACGTCAAACTGGGCAAGACCTTATCG
CGAGCGGCAAACAAAACTTAGAGGTATTACCGGACAATGAAAGCAGGTCCCTATTAGAAGGTATCGC
AAACTACTTAGTAGAAAGAGACTATTAA
Seq. ID NO: 45
>rkGPPS21
ATGTCAATGCTTATGACGCTGGTCGATGAGATCAAAAATCGTTCCAGCCATGTAGATGCAGCTATAGA
TGAATTGCTTCCCGTGACGCGTCCTGAAGAGCTGTATAAGGCTTCAAGGTATCTTGTGGACGCTGGAG
GAAAGCGTCTAAGGCCGGCCGTCCTAATTCTGGCCGCGGAGGCAGTCGGGTCCAATCTTAGGTCCGTC
CTACCCGCCGCCGTTGCGGTAGAACTTGTTCACAACTTTACGCTAATACATGACGACATTATGGATAG
AGATGACATTCGTCGTGGAATGCCCGCCGTTCATGTTAAGTGGGGTGAAGCAGGCGCGATTCTAGCGG
GGGATACCCTATATTCAAAAGCGTTTGAGATTCTATCAAAGGTGGAAAACGAGCCTGTAAGAGTACTG
AAGTGCATGGACGTTTTATCCAAGACTTGCACAGAGATTTGTGAAGGTCAATGGCTGGACATGGACTT
TGAGACTAGGAAAAAGGTTACCGAGAGCGAATATCTGGAGATGGTCGAGAAGAAGACCTCTGTACTG
TATGCGGCGGCCGCCAAAATTGGAGCGTTGCTTGGAGGGGCCTCCGATGAGGTGGCAGAGGCCCTAA
GTGAATATGGAAGGCTTATTGGAATTGGGTTCCAGATGTACGATGATGTCTTAGACATGACCGCTCCA
GAGGAGGTGTTAGGAAAGGTAAGGGGGTCTGACTTGATGGAAGGTAAGTATACTTTAATCGTGATCA
ATGCCTTCGAGAAGGGCGTTAAGTTGGACATATTTGGGAAGGGCGAAGCGACCCTAGAAGAGACCGA
AGCCGCCGTAAGAACCCTTACAGAATGTGGAAGCCTAGATTATGTAAAGAATCTAGCGATTAGTTACA
TCGAGGAAGGTAAGGAAAAGTTAGACGTGCTTAGAGATTGTCCAGAAAAGACACTTCTGTTGCAGATC
GCAGATTATATGATCTCCCGTGAGTACTAA
Seq. ID NO: 46
>rkGPPS22
ATGTCAACCGAGGTCCTGGATATACTGAGAAAGTACTCAGAAGTCGCCGACAAAAGAATAATGGAGT
GTATTTCTGACATCACACCAGATACTTTGCTTAAGGCGAGCGAACACCTAATAACGGCGGGCGGGAAG
AAAATACGTCCCTCCCTGGCCCTGCTATCATGTGAGGCAGTGGGGGGGAACCCTGAAGACGCCGCTGG
CGTAGCCGCAGCCATCGAGCTTATACATACATTTAGTTTGATTCACGACGACATAATGGATGATGACG
AGATGAGAAGGGGCGAACCCTCTGTGCATGTCATTTGGGGGGAACCAATGGCTATCTTGGCGGGAGAT
GTTCTTTTCTCTAAGGCCTTTGAAGCGGTTATCAGGAACGGCGATTCTGAGCGTGTGAAAGACGCACT
GGCTGTAGTAGTCGACAGCTGCGTCAAGATATGTGAAGGGCAGGCGCTGGATATGGGGTTCGAGGAA
AGACTAGACGTGACGGAAGATGAATACATGGAGATGATCTATAAAAAAACCGCAGCACTGATTGCTG
CTGCAACTAAAGCCGGGGCCATCATGGGGGGTGCGTCCGAACGTGAGGTGGAAGCTCTTGAGGACTA
TGGTAAATTCATCGGTTTGGCCTTTCAGATCCATGATGATTACCTTGACGTTGTCTCAGACGAGGAGAG
CCTGGGGAAACCGGTCGGGAGTGACATAGCAGAAGGTAAAATGACTTTAATGGTCGTAAAAGCGTTG
GAGGAGGCTTCAGAGGAGGATAGGGAACGTCTAATTTCCATCCTTGGTTCTGGAGATGAAGGCAGCGT
TGCCGAGGCCATCGAAATATTTGAAAGGTACGGGGCTACGCAGTATGCACACGAGGTTGCTTTAGACT
ACGTCAGGATGGCAAAAGAACGTCTTGAAATCCTAGAAGACTCTGACGCGCGTGACGCCTTGATGCGT
ATCGCGGATTTCGTGTTAGAGAGGGAGCACTAA
Seq. ID NO: 47
>bkGPPS1
MSSDSSSIGAIETRIRELVHDYVGVNGTDAPITPALRPMFHTVVDQALASSEGGKRLRALLTLDAYDVLAG
APDSTQSRSVRTKVLDFACAIEVFQTAALVHDDLIDDSDLRRGKPSAHCALTSFAGARSIGRGLGLMLGDM
LATACTLIMEDASTGMVEHRRLVEAFLSMQHDVEVGQVLDLAIERMPLDDPQALAEASLDVFRWKTASY
TTIAPLMLAFLASGMTSEAANLHCHAIGLPLGQAFQLADDLLDVTGSSRSTGKPVGGDIREGKRTVLLADA
MMLGTAAQRVQLQQLYEQPFRSDAQVHETIALFHDTGAIEHSHERIAKLWSQTQESIEAMGLTAAQSQSLR
KACERFLPDFTAER*
Seq. ID NO: 48
>bkGPPS2
MSCTTANNREIIEPRIIQLVRELTAAPATDEVADALKPVMEQVVDQAASSSQGGKRLRALLALDAFDILAG
DVTPDRRDAMIDLACAIEVFQTAALVHDDIIDESDLRRGKPSAHHALEQAVHSGAIGRGLGLMLGDILATA
CIEITRRSASRLPNTDALNEAFLTMQREVEIGQVLDLAVEMTPLSNPEALANASLNVFRWKTASYTTIAPLL
LALLAAGESPDQARHCALAVGRPLGLAFQLADDLLDVVGSSRNTGKPVGGDIREGKRTVLLADALSAADT
ADKADLIAIFEEDCRNDNQVARTIELFTSTGALDRSRERIAALWGESRKAIAGLELNSEAQRRLTEACARFV
PESLR*
Seq. ID NO: 49
>bkGPPS3
MSDKIKKMGEEIELWLKEYLDNKGNYDKKIYEAMAYSLEAGGKRIRPVLFLNTYSLYKEDYKKAMPIAAA
IEMIHTYFLIHDDLPAMDNDDLRRGKPTNHKIFGEAIAILAGDALLNEAMNIMFEYSLKNGEKALKACYTIA
KAAGVDGMIGGQVVDILSEDKSISLDELYYMHKKKTGALIKASILAGAILGSATYTDIELLGEYGDNLGLAF
QIKDDILDVEGDTTTLGKKTKSDEDNHKTTFVKVYGIEKCNELCTEMTNKCFDILNKIKKNTDKLKEITMFL
LNRNY*
Seq. ID NO: 50
>bkGPPS4
MSKKRKTLEDTAMNINSLKEEVDQSLKAYFNKDREYNKVLYDSMAYSINVGGKRIRPILMLLSYYIYKSD
YKKILTPAMAIEMIHTYFIHDDLPCMDNDDLRRGKPTNHKVFGEAIAVLAGDALLNEAMKILVDYSLEEGK
SALKATKIIADAAGSDGMIGGQIVDIINEDKEEISLKELDYMHLKKTGELIKASIMSGAVLAEASEGDIKKLE
GFGYKLGLAFQIKDDILDVVGNAKDLGKNVHKDQESNKNNYITIFGLEECKKKCVNITEECIEILSSIKGNTE
PLKVLTMKLLERKF*
Seq. ID NO: 51
>bkGPPS5
MSDFPQQLEACVKQANQALSRFIAPLPFQNTPVVETMQYGALLGGKRLRPFLVYATGHMFGVSTNTLDAP
AAAVECIHAYFLIHDDLPAMDDDDLRRGLPTCHVKFGEANAILAGDALQTLAFSILSDADMPEVSDRDRIS
MISELASASGIAGMCGGQALDLDAEGKHVPLDALERIHRHKTGALIRAAVRLGALSAGDKGRRALPVLDK
YAESIGLAFQVQDDILDVVGDTATLGKRQGADQQLGKSTYPALLGLEQARKKARDLIDDARQSLKQLAEQ
SLDTSALEALADYIIQRNK*
Seq. ID NO: 52
>bkGPPS6
MSTNFSQQHLPLVEKVMVDFIAEYTENERLKEAMLYSIHAGGKRLRPLLVLTTVAAFQKEMETQDYQVAA
SLEMIHTYFLIHDDLPAMDDDDLRRGKPTNHKVFGEATAILAGDGLLTGAFQLLSLSQLGLSEKVLLMQQL
AKAAGNQGMVSGQMGDIEGEKVSLTLEELAAVHEKKTGALIEFALIAGGVLANQTEEVIGLLTQFAHHYG
LAFQIRDDLLDATSTEADLGKKVGRDEALNKSTYPALLGIAGAKDALTHQLAEGSAVLEKIKANVPNFSEE
HLANLLTQLQLR*
Seq. ID NO: 53
>bkGPPS7
MSSSPNLSFYYNECERFESFLKNHHLHLESFHPYLEKAFFEMVLNGGKRFRPKLFLAVLCALVGQKDYSNQ
QTEYFKIALSIECLHTYFLIHDDLPCMDNAALRRNHPTLHAKYDETTAVLIGDALNTYSFELLSNALLESHII
VELIKILSANGGIKGMILGQALDCYFENTPLNLEQLTFLHEHKTAKLISASLIMGLVASGIKDEELFKWLQAF
GLKMGLCFQVLDDIIDVTQDEEESGKTTHLDSAKNSFVNLLGLERANNYAQTLKTEVLNDLDALKPAYPL
LQENLNALLNTLFKGKT*
Seq. ID NO: 54
>bkGPPS8
MSPINARLIAFEDQWVPALNAPLKQAILADSHDAQLAAAMTYSVLAGGKRLRPLLTVATMRSLGVTFVPE
RHWRPVMALELLHTYFLIHDDLPAMDNDALRRGEPTNHVKFGAGMATLAGDGLLTLAFQWLTATDLPAT
MQAALVQALATAAGPSGMVAGQAKDIQSEHVNLPLSQLRVLHKEKTGALLHYAVQAGLILGQAPEAQWP
AYLQFADAFGLAFQIYDDILDVVSSPAEMGKATQKDADEAKNTYPGKLGLIGANQALIDTIHSGQAALQGL
PTSTQRDDLAAFFSYFDTERVN*
Seq. ID NO: 55
>bkGPPS9
MSDTKILKLEDFLTEFYESAEFPTGLAESAKYSLLAGGKRIRPLLFLNLLEAFDLELSKAHYHVAAALEMIH
TGSLIHDDLPAMDNDDYRRGQLTNHKKFDEATAILAGDTLFFDPFFILSTADLSAEIIVALTRELAFASGSYG
MVAGQILDMAGEGKELTLAEIEQIHRLKTGRLLTFPFVAAGIVAQKSTDEVEKLRQVGQILGLAFQIRDDIL
DVTATFAELGKTPGKDILEEKSTYVAHLGLEGAKKSLTGNLSEVKKLLTDLSVTDSSEIFKIIEQLEVK*
Seq. ID NO: 56
>bkGPPS10
MSIDLKSFQKEWLPKINQQLENDLSMASPDADLVAMMKYAVLNGGKRLRPLLTLAVVTSFGESITPSILKV
ATAIEWVHSYFLVHDDLPAMDNDMFRRGKPSVHALYGEANAILVGDALLTGAFGVIATANSSCSVEDCLP
TEELLLITQNLAREAGGSGMVLGQLHDMDNHTEEQNASTNWLLNDVYSMKTAALIRYTTTLGAILTHQNV
NVEDNHFDPKKAMYDFGEKFGLAFQIQDDLDDYQQDQLEDVNSLPHIVGVKEAQSVLDQYLFSTQEILAN
TVEQDQQFDRRLLDDFVSLIGDKK*
Seq. ID NO: 57
>bkGPPS11
MSQDLTLFLEQYKKVIDESLFKEISERNIEPRLKESMLYSVQAGGKRIRPMLVFATLQALKVNPLLGVKTAT
ALEMIHFTYFLIHDDLPAMDNDDYRRGKYTNHKVFGDATAILAGDALLTLAFSILAEDENLSFETRIALINQI
SFSSGAEGMVGGQLADMEAENKQVTLEELSSIHARKTGELLIFAVTSAAKIAEADPEQTKRLRIFAENIGIGF
QISDDILDVIGDETKMGKKTGVDAFLNKSTYPGLLTLDGAKRALNEHVAIAKSALSGHDFDDEILLKLADLI
ALREN*
Seq. ID NO: 58
>bkGPPS12
MSTGAITEQLRRYLHDRRAETAYIGDDYSGLIAALEEFVLNGGKRLRPAFAYWGWRAVATEAPDDQALLL
FSALELLHACALVHDDVIDDSATRRGRPTTHVRFASLHRDRQWQGSPERFGMSAAILLGDLALAWADDIV
LGVDLTPQAARRVRRVWANIRTEVLGGQYLDIVAEASAAASIASAMNVDTFKTACYTVSRPLQLGAAAAA
DRPDVHDLFSQFGTDLGVAFQLRDDVLGVFGDPAVTGKPSGDDLRSGKRTVLLAEAVELAEKSDPLAAKL
LRDSIGAQLSDAEVDRLRDVIESVGALAAAEQRIATLTQRALATLAAAPINTAAKAGLSELAKLATNRSA*
Seq. ID NO: 59
>bkGPPS13
MSIPAVSLGDPQFTANVHDGIARITELINSELSQADEVMRDTVAHLVDAGGTPFRPLFTVLAAQLGSDPDG
WEVTVAGAAIELMHLGTLCHDRVVDESDMSRKTPSDNTRWTNNFAILAGDYRFATASQLASRLDPEAFAV
VAEAFAELITGQMRATRGPASHIDTIEHYLRVVHEKTGSLIAASGQLGAALSGAAEEQIRRVARLGRMIGA
AFEISRDIIAISGDSATLSGADLGQAVHTLPMLYALREQTPDTSRLRELLAGPIHDDHVAEALTLLRCSPGIG
KAKNVVAAYAAQAREELPYLPDRQPRRALATLIDHAISACD*
Seq. ID NO: 60
>bkGPPS14
MSKFKDFSNRYLPEINNDLSNYFADRDDDIFRMITYALNSTGKRLRPLLTLATFAAAGNVINDSTIEAATAV
EFVHAYFLVHDDLPEMDDDTKRRNQSSTWKKFGVGNAVLVGDGLLTEAFKKISNLSLPESIRLRLIYNLAL
AAGPDNMVRGQQYDLFSQDKVESIDDLEFIHLMKTGALMTYAATAGGILAGLSDDKLRALNIYGANLGIA
FQIKDDLRDIKQDEEENKKSFPRLIGVQKSQTELEEHLKISANAIKEIPDFQNTVLLDLLDRI*
Seq. ID NO: 61
>bkGPPS15
MSEAVLSAGAGESTRPSPSVPPFTDTVEDALREFFASRAGTVETVGGGYAEAVAALESFVLRGGKRVRPMF
VWTGWLGAGGDATGPEAPAALRAASALELVQACALVHDDIIDASTTRRGFPTVHVEFADQHSAHHWSGG
SAEFGRAVAILLGDLALAWADDMIREAGLSPDAQARISPVWSAMRTEVLGGQFLDISSEVRGDETVEAALR
VDRYKTAAYTIERPLHLGAALAGADDALVAAYRTFGTDIGIAFQLRDDLLGVFGDPEITGKPSGDDLRAGK
RTVLFAEALQRADASDPAAAALLRESIGTDLSDAQVATLRSVITDLGAVDDAERRISELTDSALSALDGSTA
TDEGKLRLREMAIAVTRRDA*
Seq. ID NO: 62
>bkGPPS16
MSDFPQQLEACVKQANQALSRFIAPLPFQNTPVVETMQYGALLGGKRLRPFLVYATGHMFGVSTNTLDAP
AAAVECIHAYFLIHDDLPAMDDDDLRRGLPTCHVKFGEANAILAGDALQTLAFSILSDANMPEVSDRDRIS
MISELASASGIAGMCGGQALDLDAEGKHVPLDALERIHRHKTGALIRAAVRLGALSAGDKGRRALPVLDK
YAESIGLAFQVQDDILDVVGDTATLGKRQGADQQLGKSTYPALLGLEQARKKARDLIDDARQALKQLAEQ
SLDTSALEALADYIIQRNK*
Seq. ID NO: 63
>bkGPPS17
MSKDKIKYINQAIKHYYAQTHVSQDLVEAVLYSVAAGGKRIRPLLLLEILQGFGLVLTEAHYQVAASLEMI
HTGFLVHDDLPAMDNDDYRRGQLTNHKKFGETTAILAGDSLFLDPFGLLAKADLRADIKIKLVAELSDAA
GSYGMVGGQMLDIKGEHVQLNLDQLAQIHANKTGKLLTFPFVAAGIIAELSEKALARLRQVGELVGLAFQ
VRDDILDVTASFSELGKTPQKDIEADKSTYPSLLGLDKSYAILEDSLNQAQAIFQKLALEEQFNATGIETIIER
LRLHA*
Seq. ID NO: 64
>bkGPPS18
MSQEALISFQQRNNQQLEWWLSQLPHQNQTLIEAMRYGLLLGGKRARPFLVYITGQMLGCKAEDLDTPAS
AVECIHAYSLIHDDLPAMDDDELRRGQPTCHIKFDEATAILTGDALQTLAFSILADGPLNPNAESMRINMVK
VLAQASGAAGMCMGQALDLQAENRLVNLQELEEIHRNKTGALMKCAIRLGALAAGEKGREVLPLLDKYA
DAIGLAFQVQDDILDIISDTETLGKPQGSDQELNKSTYPALLGLEGAIEKANNLLQEALQALDAIPYNTELLE
EFARYVIERKN
Seq. ID NO: 65
>bkGPPS19
MSHKPVDLTDTAAFETQLDRWRGRIGEAVAEAMAFGTTVPAPLQAGMSHAVLAGGKRYRGMLVLALGS
DLGVPEEQLLSSAVAIETIHAASLVVDDLPCMDDARRRRSQPATHVAFGEATAILSSIALIARAMEVVARDR
QLSPASRSSIVDTLSHAIGPQALCGGQYDDLYPPYYATEQDLIHRYQRKTSALFVAAFRCPALLAEVDPETL
LRIARAGQRLGVAFQIFDDLLDLTGDAHAIGKDVGQDHGTVTLATLLGPARAAERAADELAAVQKELRET
VGPGRALDLIRRMAARIAGTGKKSAGRDDLRPHAG
Seq. ID NO: 66
>bkGPPS20
MSAFEQRIEAAMAAAIARGQGSEAPSKLATALDYAVTPGGARIRPTLLLSVATRCGDSRPALSDAAAVALE
LIHCASLVHDDLPCFDDAEIRRGKPTVHRAYSEPLAILTGDSLIVMGFEVLAGAAADRPQRALQLVTALAV
RTGMPMGICAGQGWESESQINLSAYHRAKTGALFIAATQMGAIAAGYEAEPWEELGARIGEAFQVADDLR
DALCDAETLGKPAGQDEIHARPSAVREYGVEGAAKGLKDILGGAIASIPSCPAEAMLAEMVRRYADKIVPA
QVAARV
Seq. ID NO: 67
>bkGPPS21
MSALTLPDAQPPTGLLPLEQAWLQLVQTEVETSLAELFELPDEAGLDVRWTQALTQARAYTLRPAKRLRP
ALVMAGHCLARGSAVVPSGLWRFAAGLELLHTFLLIHDDVADQAELRRGAPPLHRMLAPGRAGEDLAVV
VGDHLFARALEVMLGSGLTCVAGVVQYYLGVSGHTAAGQYLDLDLGRAPLAEVTLFQTLRVAHLKTARY
GFCAPLVCAAMLGGASSGLVEELERVGRHVGLAYQLRDDLLGLFGDSNVAGKAADGDFLQGKRTFPVLA
AFARATEAERTELEALWALPVEQKDAAALARARALVESCGGRAACERMVVRASRAARRSLQSLPNPNGV
RELLDALIARLAHRAA
Seq. ID NO: 68
>bkGPPS22
MSEATLSAGTARVGQSSTNTAPHPTSLELPGVFEGALRDFFDSRRELVSNIGGGYEKAVSTLEAFVLRGGK
RVRPSFAWTGWLGAGGDPNGSGADAVIRACAALELVQACALVHDDIIDASTTRRGFPTVHVEFEDQHRGE
EWSGDSAHFGEAVAILLGDLALAWADDMIRESGISPDAAARVSPVWSAMRTEVLGGQFLDISNEARGDET
VEAAMRVNRYKTAAYTIERPLHLGAALFGADAELIDAYRTFGTDIGIAFQLRDDLLGVFGDPSVTGKPSGD
DLIAGKRTVLFAMALARADAADPAAAELLRNGIGTQLTDNEVDTLRQVITDLGAVTDVETQIDTLVEAAA
NALDSSTATAESKARLTDMAIAATKRSY
Seq. ID NO: 69
>bkGPPS23
MSPAGALAPLADFFAAGGKRLRPTLCVLGWHAAGGQTPASREVVQVAAALEMFHAFALIHDDVMDDSDI
RRGAPTLHRALAGQYADHRPRALTDRLGAGAAILIGDLALCWSDELIHTAGLRHDQFARILPVLDMMRTE
VMYGQYLDVTATGQPTADIGRAQTIIRYKTAKYTIERPLQLGAELAGASTDVIDALSAYAVPLGEAFQLRD
DLLGAFGDPVVTGKSSTEDLREGKPTVLVGLALRDAAPDQADVLRRLLGRRDLTEDQATQIRAVLTGTGA
RAQVENMIAQRRERVLALLDTNTVLDATAVFHLRQLADSATRRTS
Seq. ID NO: 70
>bkGPPS24
MSTVCAKKHVHLTRDAAEQLLADIDRRLDQLLPVEGERDVVGAAMREGALAPGKRIRPMLLLLTARDLG
CAVSHDGLLDLACAVEMVHAASLILDDMPCMDDAKLRRGRPTIHSHYGEHVAILAAVALLSKAFGVIADA
DGLTPLAKNRAVSELSNAIGMQGLVQGQFKDLSEGDKPRSAEAILMTNHFKTSTLFCASMQMASIVANASS
EARDCLHRFSLDLGQAFQLLDDLTDGMTDTGKDSNQDAGKSTLVNLLGPRAVEERLRQHLQLASEHLSAA
CQHGHATQHFIQAWFDKKLAAVS
Seq. ID NO: 71
>rkGPPS1
MSELDKYFDEIIKNVNEEIEKYIKGEPKELYDASIYLLKAGGKRLRPLITVASSDLFSGDRKRAYKAAAAVEI
LHNFTLIHDDIMDEDTLRRGMPTVHVKWGVPMAILAGDLLHAKAFEVLSEALEGLDSRRFYMGLSEFSKS
VIIIAEGQAMDMEFENRQDVTEEEYLEMIKKKTAQLFSCSAFLGGLVSNAEDKDLELLKEFGLNLGIAFQIID
DILGLTADEKELGKPVYSDIREGKKTILVIKALSLASEAERKIIIEGLGSKDQGKITKAAEVVKSLSLNYAYE
VAEKYYQKSMKALSAIGGNDIAGKALKYLAEFTIKRRK*
Seq. ID NO: 72
>rkGPPS2
MSTHVPANAVPTTNGLSIIPPGLSLPTTFAPLVERIQTVAHLVETAIAEDLSEVTQPELRQAVLHLFDGKGKR
LRPFLVITTAEAAGGTLEAALPPALAVEYLHNLSLIHDDMMDGSPERHGRPTLHTRFGLNLSLLVGDLLYA
KAVEQASRIRHHALRMVHILGQTAKQMCYGQFDDLYFERRLDLTIEDYLRMAARKTSALYRASCIFGMLT
ADADEADLQAMATFGENIGTAFQIWDDVLDLQADPLRLGKPLGLDIREGKKTLIVIHFLQHASPAARRRFL
ELLGKRDLNGELPEAIALLEETGSIAFARDLAIRYLVDAKQHLSVLPAGPHRKLLDMYADFMLQRRH*
Seq. ID NO: 73
>rkGPPS3
MSTSETKEARVLDAIRERRDLVNAAIDEELPVQEPERLYEATRYILEAGGKRLRPTVTTLAAEAVTGTEPM
GADFRAFPSLDGDDVDVMRAAVAIEVIQSFTLIHDDIMDEDDLRRGVPAVHEAYDVSTAILAGDTLYSKAF
EFMTETGADPQNGLEAMRMLASTCTEICEGQALDVSFESRDDILPEEYLEMVELKTAVLYGASAATPALLL
GADEEVVDALYRYGIDSGRAFQIQDDVLDLTVPSEELGKQRGSDLVEGKETLITLHARQQGIDVDGLVEAD
TPAEVTEAAIEEAVATLAEAGSIEYARETAEDLTARSKGHLEVLPESGSRSLLEDLADYLIVRGY*
Seq. ID NO: 74
>rkGPPS4
MSETLTRYLSEFRPLVDKKIMEVLEGSPKELYEAARHLPSKGGKRLRPALVLLVNKALGGEVEGALPAAA
AVELLHNFTLVHDDIMDRDELRRGVPTVHVLYGESMAILAGDLLYAKAYEALLQSPQPPDLVKEMTEVLT
WSAVTVAEGQAMDMEFEKRWDVTEEEYLEMIEKKTGALFGASAALGALTANKREVKDLMKEFGLILGK
AFQIKDDVLSLLGDEKVTGKPKYNDLREGKKTILVIYALRNLPRDEAERVKSVLGRETSYEALEEVAELIKR
SGALDYAMKLAEEFEKRAYEILETVRFEDEEAMRALKELVDFAVKREY*
Seq. ID NO: 75
>rkGPPS5
MSGKQFNLLREKYLPQIEREIKKFFEEKISTQKDEVIVRYYEELSSYVLRGGKRFRPLALISSYYGSGSKHEG
NIIRASISVELLHNSSLIHDDIMDESPKRRGGPSFHYLMANWSRLSPRTPPPRNPGISLGILGGDSLIELGLEAL
LESGFPNEIIVKAASEYSVAYRKLIEGQLLDLYLSTVTMPTEEEVLRMLSLKTGTLFSASLVMGGMLAGASE
DMLHFLRSFGQRVGVAFQLQDDILGLYGDEAVIGKPADSDIKEGKRTLLVVKAWELSDEATRKKLLSILGN
PNISAADLNYVREVVKELGALDYTRKTALNLLKENEKDIEFNKHLFEESFVEFLKELNEIVIARSF*
Seq. ID NO: 76
>rkGPPS6
MSSNINEDVGKVLGQYSKDIHKEIGNTLSNIGPEDLREASIYLTEAGGKMLRPALTVLICEAVGGTFSSCIKA
AAAIELIHTFSLIHDDIMDKDDMRRGKPSVHKVWGEPVAILAGDTLFSKAYELVINSKNEIDSSNPEECLNR
VNRTLSTVADACVKICEGQAQDMGFEGNFDVSEEEYMEMIFKKTAALIAAATESGAIMGGANEKIVSDMY
DYGKLIGLAFQIQDDYLDLVSDEDSLGKPVGSDIAEGKMTIIVVNALNRANPEDKKRILEILRMGNESGNCD
QVYVDEAISLFEKYGSIQYAQNIALANVKKAKQLLEILPESEAKHTLSLVADFVLYRQN*
Seq. ID NO: 77
>rkGPPS7
MSSDLKTYLEKTAEQVDIALERNFGDVFGDLYKASAHLLLAGGKRLRPAVLLLAANAVKPGRADDLITAA
IAVEMTHTFYLIHDDIMDGDVTRRGVPTVHTKWDEPTAILAGDVLYAKSFEYITHALAEDRARVKAVTLL
ARTCTEICEGQHQDMAFEQKGAEVEEADYIEMAGKKTGALYAAAAAIGGTLAGGNAMQVDALYQYGMN
AGIAFQIQDDLIDLLAPPETSGKDRASDLREGKQTLIAIIAREKDLDLSKYRHTLTTTEIDAAIAELEGAGVVD
EVRRAAEERVATAKRALSVLPESMERTYLEEIADYFLTRSF*
Seq. ID NO: 78
>rkGPPS8
MSDLIDELKKRSTLVDESIQEFLPIDHPEELYRATRYLPDAGGKRLRPAVLMLSAEAVGGDSDSVLPAAVAL
ELIHNFTLIHDDIMDRDDIRRGMPALHVKWGTAGAILAGDTLYSRAFEIISKMDADPQKLLKCVALLSRTCT
KICEGQWLDVDFEKRDIVDVDEYLEMIENKTSVLYGAAAKVGAILGGASDEVADAMYEFGRLTGISFQIHD
DVIDLVTPEEILGKSRGSDLKEGKKTLIALHALNNGVELECFGKADATQDEINNAVAKLEESGTLAYVREM
ADNYLEDGKSKLDLLEDSPAKETLIEIADYMVSREY*
Seq. ID NO: 79
>rkGPPS9
MSDLIEEIKKRSSHVDKGIEEYLPIDKPYELYKAARYLPDAGGKRLRPATVILAAEAVGSDLETVLPAAVAV
ELVHNFYLVHDDIMDRDDIRRGMPAVHVKWGEAGAILAGDTLYSKAFEILTHAPAEAPERNLKCIDILSKA
CRDICEGQWMDVEFENRDDVTKEEYLEMIEKKTGVLYAASMQIGAILGGAPEEVSDAFYECGRLIGIAFQI
YDDVIDMTTPEEVLGKVRGSDLMEGKKTLIAIHALNKGVELKIFGKGEATTEEINEAVHQLEEAGSIDYVR
DLALDYIARGKELLNVVEDSESKTILKAIADYMITRSY*
Seq. ID NO: 80
>rkGPPS10
MSIEEILQKKAKLVDESIPKFLPITPPDELYKAMRHLLDAGGKRLRPSALLLASEAVGGKPDDVLPAAVAVE
LVHNFTLIHDDIMDEADLRRGLATVHKKWGVPRAIIAGDALYSKAFEILSCTKSEPQRLVESLELLSKTCTDI
CEGQWMDMNFQTRKDVTEEEYMRMVEKKTAVLFATALKLGAVLSGANREHVRALWDFGRLTGVGFQIY
DDVIDLITPEEILGKAQGGDIIEGKRTLIIIHALSKGISIDALGKCNATRSEISAALTTLKESGSIDYAMNKALSF
VDEGKAALAMLPESEAKNILTRLADYMIERKY*
Seq. ID NO: 81
>rkGPPS11
MSEADMSDLSAYLKSVAQQIDGMIEKNFTHAGGELDRASAHLLSAGGKRLRPAVVMLSADAIRHGSSKDV
MPAALALEVTHTFYLIHDDIMDGDSLRRGVPTVHTKWDMPTGILAGDVLYARAFEFICQSKADEGPKVQA
VALLARACADICEGQHQDMSFEHRADVTEEEYMAMVAKKTGVLYAAAAAIGGTLAGGNPEQIRALYQFG
LNTGIAFQIQDDLIDLLTPTEKSGKDQGSDLREGKQTLVMIIARQKGVDLLKYRHELSPADIKAAIQELTDA
GVIDAVKKKAADLVADSNRLLMVLPPTKERQLIMDVGEFFVTRSF*
Seq. ID NO: 82
>rkGPPS12
MSELIEYLEKVGNQVDRLIDRYFGDPVGELNKASAHLLTAGGKRLRPAVMMLAADAVRKGSSDDLMPAA
IALELTHSFYLIHDDIMDGDEVRRGVPTVNKKWDEPTAILAGDVLYARAFAFICQALAMDAAKLRAVSML
AVTCEEICAGQHLDMAFEDRDDVSEEEYLEMVGKKTGALYAASTAMGGVLAGGSQPQVDALYRYGMNI
GVAFQIQDDLIDLLASPERSGKDRASDIREGKQTLINIKAREHGFDLAPYRRRLDDAEIDDLIQQLTDNGVIG
EVKATAEGLVTSAGKILAILKPSDEKDLLISIGTFFVERGY*
Seq. ID NO: 83
>rkGPPS13
MSKDTTKIEVENYINKVNNHLISFLSGKPLQLYQASTHYLKSGGKRLRPIMVIKSCEMFGGTQQDALPAAA
AVEFIHNFSLVHDDIMDNDDLRHGIPTVHKSFGLPLAILSGDILFSKAFQILSITNVNSIKDSSLLSMIRRLSLA
CVDICEGQAKDIQFSECETFPSEEEYLEMISKKTAALFNVSCSLGALSSRNATEKDVNNMSDFGKNSGIAFQ
LIDDLIGIAGHSKETGKAVGNDIREGKKTYPILLSIKKASELERAHILKVFGKGQCDNMSLKKAIDVISSLQIE
KIVRKSAMAYIEKAMEALVNYEDSEPKKILQELSSYIVERSK*
Seq. ID NO: 84
>rkGPPS14
MSLQDYFNEVINQVNKTIEKYLSNAPSGTSSLYEASKHLFSAGGKRLRPLILVSSCDFLGGDRSRAILAGSAI
ETLHTFTLIHDDIMDHDFLRRGLPTVHVKWGESMAILAGDLLHAKAFEMLNDSLEGVNETLHYEVMKTFI
NSIVVVSEGQAMDMQFEGRNDVTEEDYLEMVKKKTAYLIATSSKIGSLIGGAGPDVADKFFHFGIYLGIAF
QIVDDIIGITSDEAELGKPLFSDIREGKRTLLVIRTLKEAESRELEVLKQVLGNKNASTDQLKEASQIVKKHSL
EYAYSLAEEYRSRAISSLDGIQPRNQEAYEALKFVSEFTLKRKK*
Seq. ID NO: 85
>rkGPPS15
MSSFNSISKTAKKVNSFLLSSLHGNPEEIYKAASYLIEYGGKRLRPYMVIKSCEILGGTIKQALPSAAAIEMV
HNFTLIHDDIMDNDEIRHGVSTTHKKFGIPVGILAGDVLFSKAFETISHGDPKMPKDVRLALVSNLAKACTD
VCEGQALDIMMAKSQKIPTEEQYIMMIEKKTSALFAAACAMGAISANTKTRDVTNLSSFGKNLGVAFQIVD
DLIGIIGDSKITKKPVGNDLREGKKSLPILLAINKVSGKKKEIILNAFGNSAISKKELENAVRIISSMGIETAVR
KKAIQYSNAAKKSLSNYKGSAKNELLSLLDFVVERSQ*
Seq. ID NO: 86
>rkGPPS16
MSGKYDELFAQVKAKAKDVDAVIFELIPEKEPKTLYEAARHYPLAGGKRVRPFVVLRAAEAVGGDPEKAL
YPAAAVEFIHNYSLVHDDIMDMDELRRGRPTVHKLWGVNMAILAGDLLFSKAFEAVARAEVSPEKKARIL
DVLVKTSNELCEGQALDIEFETRDEVTVDEYLKMISGKTGALFNGSATIGAIVGTDNEKYIQALSKWGRNV
GIAFQIWDDVLDLIADEEKLGKPVGSDIRKGKKTLIVSHFFQHANEEDKAEFLKVFGKYAGDAKGDALIHD
EKVKEEVAKAIELLKKYGSIDYAANYAKNLVREANEALKVLPESEARKDLELLAEFLVEREF*
Seq. ID NO: 87
>rkGPPS17
MSDIISRFSEKIDAVNSAIDKFLRIREPKRLYSATRHLPLAGGKRLRPILAMLSTEAVGEDWKKTIPFAVSLEL
LHNFTLVHDDIMDRSDLRRGIETVHVKFGEPTAILAGDILFAKSFEVLYELDIDDAIFKTVNRLLIDCIEEICD
GQQIDMEFESRKYVSEEEYLEMIEKKTSALFSCATTGGAIIGDGNNREVDSLSLYGRFFGLAFQIWDDYLDI
AGEEGEFGKKIGNDIRCGKKTLMIVHATKNADGREKETIFSILGKKDATDEEINEVMEILTKSGSIDYAKKK
ALHFAEKAKEQLRVLPDSRAKRDLIELVDFAISRER*
Seq. ID NO: 88
>rkGPPS18
MSLIDHYIMDFMSITPDRLSGASLHLIKAGGKRLRPLITLLTARMLGGLEAEARAIPLAASIETAHTFSLIHDD
IMDRDEVRRGVPTTHVVYGDDWAILAGDTLHAAAFKMIADSREWGMSHEQAYRAFKVLSEAAIQISRGQ
AYDMLFEETWDVDVADYLNMVRLKTGALIEAAARIGAVAAGAGSEIEKMMGEVGMNAGIAFQIRDDILG
VIGDPKVTGKPVYNDLRRGKKTLLVIYAVKKAGRREIVDLIGPKASEDDLKRAASIIVDSGALDYAESRARF
YVERARDILSRVPAVDAESKELLNLLLDYIVERVK*
Seq. ID NO: 89
>rkGPPS19
MSISEIIKDRAKLVNEKIEELLKEQEPEGLYRAARHYLKAGGKRLRPVITLLSAEALGEDYRKAIHAAIAIET
VHNFTLVHDDIMDEDEMRRGVKTVHTLFGIPTAILAGDTLYAEAFEILSMSDAPPENIVRAVSKLARVCVEI
CEGQFMDMSFEERDSVGESEYLEMVRKKTGVLIGISASIPAVLFGKDESVEKALWNYGIYSGIGFQIHDDLL
DISGKGKIGKDWGSDILEGKKTLIVIKAFEEGIELETFGKGRASEEELERDIKKLFDCGAVDYARERAREYIE
MAKKNLEVIDESPSRNYLVELADYLIERDH*
Seq. ID NO: 90
>rkGPPS20
MSSERHQQVEDAIVARRDRVNDALPEDLPVKKPDHLYEASRYLLDAGGKRLRPTVLLLVAESLLDVDPLT
ADYRDFPTLGGGQADMMSAALAIEVIQTFTLIHDDIMDDDALRRGVPAVHKEYDLSTAILAGDTLYSKAFE
FLLGTGAAHERTVEANKRLATTCTRICEGQSLDIEFEQRDVVTPEEYLEMVELKTAVLYGAAASIPATLLGA
DAETVDALYNYGLDVGRAFQIQDDLLDLTTPSEKLGKQRGSDLVENKQTLVTLHARQQGVDVGDLIDTDS
VEAVSEAEIDAAVERLREVGSIEYARQTGQDLIASGKQNLEVLPDNESRSLLEGIANYLVERDY*
Seq. ID NO: 91
>rkGPPS21
MSMLMTLVDEIKNRSSHVDAAIDELLPVTRPEELYKASRYLVDAGGKRLRPAVLILAAEAVGSNLRSVLPA
AVAVELVHNFTLIHDDIMDRDDIRRGMPAVHVKWGEAGAILAGDTLYSKAFEILSKVENEPVRVLKCMDV
LSKTCTEICEGQWLDMDFETRKKVTESEYLEMVEKKTSVLYAAAAKIGALLGGASDEVAEALSEYGRLIGI
GFQMYDDVLDMTAPEEVLGKVRGSDLMEGKYTLIVINAFEKGVKLDIFGKGEATLEETEAAVRTLTECGS
LDYVKNLAISYIEEGKEKLDVLRDCPEKTLLLQIADYMISREY*
Seq. ID NO: 92
>rkGPPS22
MSTEVLDILRKYSEVADKRIMECISDITPDTLLKASEHLITAGGKKIRPSLALLSCEAVGGNPEDAAGVAAAI
ELIHTFSLIHDDIMDDDEMRRGEPSVHVIWGEPMAILAGDVLFSKAFEAVIRNGDSERVKDALAVVVDSCV
KICEGQALDMGFEERLDVTEDEYMEMIYKKTAALIAAATKAGAIMGGASEREVEALEDYGKFIGLAFQIHD
DYLDVVSDEESLGKPVGSDIAEGKMTLMVVKALEEASEEDRERLISILGSGDEGSVAEAIEIFERYGATQYA
HEVALDYVRMAKERLEILEDSDARDALMRIADFVLEREH*
Seq. ID NO: 93
>MBP
ATGAAGATCGAAGAAGGAAAGTTAGTGATCTGGATAAATGGTGATAAAGGCTACAATGGGTTGGCGG
AAGTAGGAAAAAAGTTCGAGAAAGACACAGGAATCAAAGTTACGGTCGAGCACCCCGATAAACTAGA
GGAAAAGTTTCCACAGGTAGCTGCTACGGGGGACGGACCAGACATTATCTTTTGGGCCCACGATAGAT
TCGGGGGTTATGCTCAGTCCGGACTTCTGGCCGAGATTACTCCAGACAAGGCCTTCCAAGACAAaCTTT
ACCCGTTcACaTGGGACGCAGTCAGGTACAATGGAAAGCTGATTGCATATCCGATAGCTGTGGAGGCA
CTTAGCCTAATTTACAACAAGGATCTACTACCTAACCCCCcAAGACTTGGGAAGAAATTCCAGCTCTG
GACAAGGAGTTAAAAGCAAAgGGtAAGAGTGCACTTATGTTCAATCTACAAGAGCCTTATTTCACATGG
CCCCTAATAGCCGCCGACGGAGGCTATGCCTTTAAGTACGAAAACGGCAAGTATGACATAAAGGATGT
TGGGGTAGACAACGCGGGAGCCAAGGCTGGATTAACTTTCCTGGTGGATTTAATTAAgAACAAACACA
TGAACGCAGACACTGACTACTCTATCGCAGAAGCAGCGTTCAATAAAGGCGAAACGGCGATGACAAT
TAACGGGCCCTGGGCTTGGTCAAACATTGACACGAGTAAAGTTAACTATGGTGTAACGGTATTGCCCA
CATTTAAGGGACAACCCAGTAAACCTTTCGTAGGAGTCTTGTCAGCCGGGATCAATGCAGCTTCCCCG
AATAAAGAGCTTGCTAAGGAATTTCTTGAAAATTATCTTTTAACCGATGAGGGATTGGAGGCGGTTAA
CAAGGACAAGCCTCTTGGTGCTGTAGCCCTGAAATCCTATGAAGAAGAGTTAGCTAAGGACCCAAGA
ATCGCCGCAACAATGGAGAATGCTCAGAAGGGAGAAATTATGCCAAATATACCACAAATGAGTGCCT
TCTGGTATGCGGTAAGGACGGCAGTTATTAATGCCGCTTCAGGTAGACAAACAGTCGATGAGGCTTTG
AAAGATGCACAGACTAACAGTTCATCCAAcAATAATAACAATAACAATAACAATAACCTGGGTATCGA
GGGCCGTTAA
Seq. ID NO: 94
>VEN
ATGGTATCtAAAGGAGAAGAATTGTTTACAGGcGTGGTACCAATTCTGGTTGAATTGGACGGTGACGTG
AACGGACACAAATTCAGCGTGAGTGGAGAAGGCGAGGGAGATGCTACCTATGGCAAGTTGACGCTTA
AACTGATCTGCACAACGGGCAAATTACCAGTGCCCTGGCCGACGCTTGTAACAACTCTTGGATACGGG
TTACAGTGCTTTGCCCGTTATCCAGACCATATGAAACAGCATGACTTcTTCAAATCTGCGATGCCGGAG
GGATATGTACAGGAACGTACGATTTTCTTTAAGGACGATGGGAACTACAAGACTCGTGCTGAGGTTAA
GTTTGAAGGCGACACTCTAGTCAATAGGATAGAATTAAAGGGTATTGATTTTAAGGAGGATGGGAACA
TCCTGGGCCATAAACTAGAGTACAACTACAATTCACATAATGTCTACATCACCGCTGATAAACAgAAG
AACGGGATCAAAGCTAATTTCAAGATACGTCATAATATCGAAGATGGTGGCGTCCAGCTTGCTGACCA
CTACCAGCAGAACACGCCTATAGGCGACGGGCCGGTGTTGCTACCTGACAATCATTATCTGTCCTATC
AGTCCGCCCTTTCAAAAGACCCTAATGAGAAGAGGGATCATATGGTGCTTTTAGAATTTGTAACCGCG
GCAGGGATCACACTTGGGATGGATGAGCTGTATAAA
Seq. ID NO: 95
>MST
ATGGCGATGTTCTGTACCTTCTTTGAGAAACATCATAGAAAATGGGACATCTTACTAGAAAAGAGCAC
CGGaGTGATGGAGGCGATGAAAGTAACTTCAGAAGAgAAAGAGCAGTTGTCTACAGCTATCGATAGAA
TGAATGAAGGTCTGGACGCATTTATTCAACTATATAACGAATCCGAGATCGATGAACCTTTAATCCAG
TTGGATGACGATACAGCAGAACTAATGAAACAGGCTAGGGACATGTACGGCCAAGAGAAACTTAACG
AGAAATTAAACACAATAATCAAACAAATCCTGTCAATTTCTGTCTCCGAAGAGGGTGAGAAAGAAGG
AAGCGGATCAGGC
Seq. ID NO: 96
>OSP
ATGTACCTACTTGGGATTGGACTTATTCTGGCGCTTATTGCTTGTAAGCAAAATGTTTCCAGCCTAGAT
GAAAAAAATTCCGTGTCTGTCGATCTTCCTGGCGAAATGAAGGTTTTAGTATCCAAGGAGAAAAATAA
GGACGGCAAATACGACTTGATTGCGACAGTCGATAAACTAGAGCTAAAAGGCACGAGCGATAAAAAT
AACGGCTCTGGAGTGTTAGAAGGGGTAAAAGCAGATAAAAGCAAGGTCAAGCTGACCATATCAGATG
ATGGATCAGGC
Seq. ID NO: 97
>OLE
ATGGCGGACAGGGACAGGTCAGGTATCTATGGGGGGGCTCATGCGACCTATGGGCAACAGCAGCAGC
AGGGAGGTGGTGGACGTCCGATGGGAGAACAAGTTAAGGGCATGTTACACGACAAAGGTCCCACTGC
CTCCCAAGCATTGACCGTTGCAACATTGTTCCCATTGGGCGGACTTTTATTAGTCCTTTCTGGCCTGGCT
CTAACTGCAAGCGTGGTAGGCCTAGCTGTAGCCACACCCGTGTTCTTGATTTTTTCTCCGGTCCTTGTA
CCGGCGGCTTTACTGATCGGTACTGCTGTAATGGGTTTCCTAACATCCGGGGCCTTAGGGTTAGGGGG
GTTGTCATCCTTAACCTGCCTAGCGAACACCGCCAGGCAGGCGTTTCAGCGTACTCCCGATTACGTCGA
GGAAGCCCACAGGAGAATGGCTGAGGCTGCGGCGCATGCGGGACATAAAACTGCCCAGGCAGGACAA
GCTATTCAGGGCCGTGCACAGGAGGCAGGAGCCGGCGGAGGCGCGGGA
Seq. ID NO: 98
>MBP
MKIEEGKLVIWINGDKGYNGLAEVGKKFEKDTGIKVTVEHPDKLEEKFPQVAATGDGPDIIFWAHDRFGG
YAQSGLLAEITPDKAFQDKLYPFTWDAVRYNGKLIAYPIAVEALSLIYNKDLLPNPPKTWEEIPALDKELKA
KGKSALMFNLQEPYFTWPLIAADGGYAFKYENGKYDIKDVGVDNAGAKAGLTFLVDLIKNKHMNADTD
YSIAEAAFNKGETAMTINGPWAWSNIDTSKVNYGVTVLPTFKGQPSKPFVGVLSAGINAASPNKELAKEFL
ENYLLTDEGLEAVNKDKPLGAVALKSYEEELAKDPRIAATMENAQKGEIMPNIPQMSAFWYAVRTAVINA
ASGRQTVDEALKDAQTNSSSNNNNNNNNNNLGIEGR
Seq. ID NO: 99
>VEN
MVSKGEELFTGVVPILVELDGDVNGHKFSVSGEGEGDATYGKLTLKLICTTGKLPVPWPTLVTTLGYGLQC
FARYPDHMKQHDFFKSAMPEGYVQERTIFFKDDGNYKTRAEVKFEGDTLVNRIELKGIDFKEDGNILGHKL
EYNYNSHNVYITADKQKNGIKANFKIRHNIEDGGVQLADHYQQNTPIGDGPVLLPDNHYLSYQSALSKDPN
EKRDHMVLLEFVTAAGITLGMDELYK
Seq. ID NO: 100
>MST
MAMFCTFFEKHHRKWDILLEKSTGVMEAMKVTSEEKEQLSTAIDRMNEGLDAFIQLYNESEIDEPLIQLDD
DTAELMKQARDMYGQEKLNEKLNTIIKQILSISVSEEGEKEGSGSG
Seq. ID NO: 101
>OSP
MYLLGIGLILALIACKQNVSSLDEKNSVSVDLPGEMKVLVSKEKNKDGKYDLIATVDKLELKGTSDKNNGS
GVLEGVKADKSKVKLTISDDGSG
Seq. ID NO: 102
>OLE
MADRDRSGIYGGAHATYGQQQQQGGGGRPMGEQVKGMLHDKGPTASQALTVATLFPLGGLLLVLSGLA
LTASVVGLAVATPVFLIFSPVLVPAALLIGTAVMGFLTSGALGLGGLSSLTCLANTARQAFQRTPDYVEEAH
RRMAEAAAHAGHKTAQAGQAIQGRAQEAGAGGGAG

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

1. A nucleic acid comprising a recombinant bacterial or archaeal geranyl pyrophosphate synthase (GPPS) gene, codon optimized for production in yeast.

2. The nucleic acid of claim 1, comprising a nucleotide sequence 90%, 95%, 98%, 99% or 100% identical to any one of the thirty-four sequences of SEQ ID NOs:1-46, or its complement, or an RNA equivalent thereof.

3. The nucleic acid of claim 1, encoding an enzymatically active GPPS comprising an amino acid sequence having at least 80%, at least 85%, at least 90%, at least 95%, or 100% amino acid sequence identity or conservative amino acid substitutions to any one of the thirty-four sequences of SEQ ID NOs:47-92.

4. The nucleic acid of claim 1 further comprising nucleic acids encoding amino acids that are not part of a GPPS.

5. The nucleic acid of claim 4 having a 5′ end, wherein the additional nucleic acids are at the 5′ end of the nucleic acid and encode a codon optimized cofolding peptide.

6. The nucleic acid of claim 5, wherein the codon optimized cofolding peptide comprises SEQ ID NO:98-102.

7. The nucleic acid of claim 6, wherein the codon optimized cofolding peptide is encoded by any one of SEQ ID NOs:93-97.

8. The nucleic acid of claim 1, further comprising a promoter functional in a yeast.

9. A yeast expression cassette comprising the nucleic acid of claim 8.

10. A yeast cell comprising the expression cassette of claim 9, capable of expressing a GPP synthase comprising an amino acid sequence having at least 80%, at least 85%, at least 90%, at least 95%, or 100% amino acid sequence identity or conservative amino acid substitutions to any one of the thirty-four sequences of SEQ ID NOs:35-68.

11. The yeast cell of claim 10, which is a species of Saccharomyces, Candida, Pichia, Schizosaccharomyces, Scheffersomyces, Blakeslea, Rhodotorula, or Yarrowia.

12. The yeast cell of claim 10 or 11, further comprising a second recombinant nucleic acid, wherein the second recombinant nucleic acid encodes a second enzyme in a terpenoid biosynthetic pathway, wherein the yeast cell is capable of expressing the second enzyme.

13. The yeast cell of claim 12, wherein the second enzyme catalyzes synthesis of a compound that immediately precedes or is immediately after a product of the GPPS in the terpenoid biosynthetic pathway.

14. The yeast cell of claim 13, further comprising a third recombinant nucleic acid, wherein the third recombinant nucleic acid encodes a third enzyme in the terpenoid biosynthetic pathway, wherein the yeast cell is capable of expressing the second enzyme.

15. The yeast cell of claim 14, capable of processing a compound through at least three steps in the terpenoid biosynthetic pathway.

16. The yeast cell of claim 10, wherein the terpenoid biosynthetic pathway is not a cannabinoid biosynthetic pathway.

17. The yeast cell of claim 16, capable of producing nerol, geraniol, pinene, limonene, linalool, neral, citral, myrcene, ocimene, zingiberene, patchoulol, bisabolene, humulene, camphor, sabinene, geranylgeraniol, phytol, geranyllinalool, retinol, or any combination thereof.

18. The yeast cell of claim 17, wherein the terpene is a monoterpene and the recombinant GPPS preferentially produces geranyl pyrophosphate (GPP) over farnesyl pyrophosphate (FPP) or geranylgeranyl pyrophosphate (GGPP).

19. The yeast cell of claim 17, wherein the terpene is a sesquiterpene and the recombinant GPPS preferentially produces FPP over GPP or GGPP.

20. The yeast cell of claim 17, wherein the terpene is a diterpene and the recombinant GPPS preferentially produces GGPP over GPP or FPP.

21. The yeast cell of claim 13, wherein the terpenoid biosynthetic pathway is a cannabinoid biosynthetic pathway.

22. The yeast cell of claim 21, capable of producing cannabigerolic acid (CBGA), cannabidiolic acid (CBDA), cannabichromenic acid (CBCA), cannabinerolic acid (CBNA), cannabigerolic acid (CBGA), cannabinerovarinic acid (CBNVA), cannabigerophorolic acid (CBGPA), cannabigerovarinic acid (CBGVA), cannabigerogerovarinic acid (CBGGVA), tetrahydrocannabinolic acid (THCA), cannabinerovarinic acid (CBNVA), sesquicannabigerol (CBF), cannabigerogerol (CBGG), sesqui-cannabigerolic acid (CBFA), cannabigerogerolic acid (CBGGA), sesquicannabigerolic acid (CBFA), sesquicannabidiolic acid (CBDFA), sesquiTHCA (THCFA), sesqui-cannabigerovarinic acid (CBFVA), sesquiCBCA (CBCFA), sesquiCBGPA (CBFPA) or any combination thereof.

23. The yeast cell of claim 22, wherein the GPPS preferentially produces GPP over FPP.

24. A method of producing a terpene in a yeast, the method comprising incubating the yeast cell of claim 10 in a manner sufficient to produce the terpene.

25. The method of claim 24, wherein the terpene is not a cannabinoid.

26. The method of claim 25, wherein the terpene is nerol, geraniol, pinene, limonene, linalool, neral, citral, myrcene, ocimene, zingiberene, patchoulol, bisabolene, humulene, camphor, sabinene, geranylgeraniol, phytol, geranyllinalool, thujone, salvinorin, retinol, or any combination thereof.

27. The method of claim 25, wherein the terpene is a monoterpene and the recombinant GPPS preferentially produces geranyl pyrophosphate (GPP) over farnesyl pyrophosphate (FPP) or geranylgeranyl pyrophosphate (GGPP).

28. The method of claim 25, wherein the terpene is a sesquiterpene and the recombinant GPPS preferentially produces FPP over GPP or GGPP.

29. The method of claim 25, wherein the terpene is a diterpene and the recombinant GPPS preferentially produces GGPP over GPP or FPP.

30. The method of claim 24, wherein the terpene is a cannabinoid.

31. The method of claim 30, wherein the cannabinoid is cannabigerolic acid (CBGA), cannabidiolic acid (CBDA), cannabichromenic acid (CBCA), cannabinerolic acid (CBNA), cannabigerolic acid (CBGA), cannabinerovarinic acid (CBNVA), cannabigerophorolic acid (CBGPA), cannabigerovarinic acid (CBGVA), cannabigerogerovarinic acid (CBGGVA), tetrahydrocannabinolic acid (THCA), cannabinerovarinic acid (CBNVA), sesquicannabigerol (CBF), cannabigerogerol (CBGG), sesqui-cannabigerolic acid (CBFA), cannabigerogerolic acid (CBGGA), sesquicannabigerolic acid (CBFA), sesquicannabidiolic acid (CBDFA), sesquiTHCA (THCFA), sesqui-cannabigerovarinic acid (CBFVA), sesquiCBCA (CBCFA), sesquiCBGPA (CBFPA) or any combination thereof.

32. The method of claim 30, wherein, the GPPS preferentially produces GPP over FPP.
